(19) United States
(12) Patent Application Publication
(10) Pub. No.: US 2010/0186121 A1
(43) Pub. Date: Jul. 22, 2010

(54) Transgenic Plants with Enhanced Growth Characteristics

(75) Inventors: Pat J. Unkefer, Los Alamos, NM (US); Penelope S. Anderson, Los Alamos, NM (US); Thomas J. Knight, Raymond, ME (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: Los Alamos National Security, LLC, Los Alamos, NM (US)

(21) Appl. No.: 12/551,271

(22) Filed: Aug. 31, 2009

Related U.S. Application Data
(60) Provisional application No. 61/190,520, filed on Aug. 29, 2008.

(51) Int. Cl. A01H 5/00; C12N 15/82

(52) U.S. Cl. 800/290; 800/298; 800/278

(57) ABSTRACT

The invention relates to transgenic plants exhibiting dramatically enhanced growth rates, greater seed and fruit/pod yields, earlier and more productive flowering, more efficient nitrogen utilization, increased tolerance to high salt conditions, and increased biomass yields. In one embodiment, transgenic plants engineered to over-express both glutamine phenylpyruvate transaminase (GPT) and glutamine synthetase (GS) are provided. The GPT+GS double-transgenic plants of the invention consistently exhibit enhanced growth characteristics, with T0 generation lines showing an increase in biomass over wild type counterparts of between 50% and 300%. Generations that result from sexual crosses and/or selfing typically perform even better, with some of the double-transgenic plants achieving an astounding four-fold biomass increase over wild type plants.

### RELATED APPLICATIONS

This application claims priority to U.S. Provisional Application No. 61/190,520 filed Aug. 29, 2008.

### STATEMENT AS TO RIGHTS TO INVENTIONS MADE UNDER FEDERALLY SPONSORED RESEARCH OR DEVELOPMENT

This invention was made with government support under Contract No. W-7405-ENG-36 awarded by the United States Department of Energy to The Regents of The University of California, and Contract No. DE-AC52-06NA25396, awarded by the United States Department of Energy to Los Alamos National Security, LLC. The government has certain rights in this invention.

### BACKGROUND OF THE INVENTION

As the human population increases worldwide, and available farmland continues to be destroyed or otherwise compromised, the need for more effective and sustainable agriculture systems is of paramount interest to the human race. Improving crop yields, protein content, and plant growth rates represent major objectives in the development of agriculture systems that can more effectively respond to the challenges presented.

In recent years, the importance of improved crop production technologies has only increased as yields for many well-developed crops have tended to plateau. Many agricultural activities are time sensitive, with costs and returns being dependent upon rapid turnover of crops or upon time to market. Therefore, rapid plant growth is an economically important goal for many agricultural businesses that involve high-value crops such as grains, vegetables, berries and other fruits.

Genetic engineering has and continues to play an increasingly important yet controversial role in the development of sustainable agriculture technologies. A large number of genetically modified plants and related technologies have been developed in recent years, many of which are in widespread use today (Factsheet: Genetically Modified Crops in the United States, Pew Initiative on Food and Biotechnology, August 2004, http://pewagbiotech.org/resources/factsheets). The adoption of transgenic plant varieties is now very substantial and is on the rise, with approximately 250 million acres planted with transgenic plants in 2006.

While acceptance of transgenic plant technologies may be gradually increasing, particularly in the United States, Canada and Australia, many regions of the World remain slow to adopt genetically modified plants in agriculture, notably Europe. Therefore, consonant with pursuing the objectives of responsible and sustainable agriculture, there is a strong interest in the development of genetically engineered plants that do not introduce toxins or other potentially problematic substances into plants and/or the environment. There is also a strong interest in minimizing the cost of achieving objectives such as improving herbicide tolerance, pest and disease resistance, and overall crop yields. Accordingly, there remains a need for transgenic plants that can meet these objectives.

The goal of rapid plant growth has been pursued through numerous studies of various plant regulatory systems, many of which remain incompletely understood. In particular, the plant regulatory mechanisms that coordinate carbon and nitrogen metabolism are not fully elucidated. These regulatory mechanisms are presumed to have a fundamental impact on plant growth and development.

The metabolism of carbon and nitrogen in photosynthetic organisms must be regulated in a coordinated manner to assure efficient use of plant resources and energy. Current understanding of carbon and nitrogen metabolism includes details of certain steps and metabolic pathways which are subsystems of larger systems. In photosynthetic organisms, carbon metabolism begins with CO₂ fixation, which proceeds via two major processes, termed C-3 and C-4 metabolism. In plants with C-3 metabolism, the enzyme ribulose bisphosphate carboxylase (RuBisCo) catalyzes the combination of CO₂ with ribulose bisphosphate to produce 3-phosphoglycerate, a three carbon compound (C-3) that the plant uses to synthesize carbon-containing compounds. In plants with C-4 metabolism, CO₂ is combined with phosphoenol pyruvate to form acids containing four carbons (C-4), in a reaction catalyzed by the enzyme phosphoenol pyruvate carboxylase. The acids are transferred to bundle sheath cells, where they are decarboxylated to release CO₂, which is then combined with ribulose bisphosphate in the same reaction employed by C-3 plants.

Numerous studies have found that various metabolites are important in plant regulation of nitrogen metabolism. These compounds include the organic acid malate and the amino acids glutamate and glutamine. Nitrogen is assimilated by photosynthetic organisms via the action of the enzyme glutamine synthetase (GS) which catalyzes the combination of ammonia with glutamate to form glutamine. GS plays a key role in the assimilation of nitrogen in plants by catalyzing the addition of ammonium to glutamate to form glutamine in an ATP-dependent reaction (Miflin and Habash, 2002, Journal of Experimental Botany, Vol. 53, No. 370, pp. 979-987). GS also reassimilates ammonia released as a result of photorespiration and the breakdown of proteins and nitrogen transport compounds. GS enzymes may be divided into two general classes, one representing the cytoplasmic form (GS1) and the other representing the plastidic (i.e., chloroplastic) form (GS2).

Previous work has demonstrated that increased expression levels of GS1 result in increased levels of GS activity and plant growth, although reports are inconsistent. For example, Fuentes et al. reported that CaMV S35 promoter-driven overexpression of Alfalfa GS1 (cytoplasmic form) in tobacco resulted in increased levels of GS expression and GS activity in leaf tissue, increased growth under nitrogen starvation, but no effect on growth under optimal nitrogen fertilization conditions (Fuentes et al., 2001, J. Exp. Botany 52: 1071-81). Temple et al. reported that transgenic tobacco plants overexpressing the full length Alfalfa GS1 coding sequence contained greatly elevated levels of GS transcript, and GS polypeptide which assembled into active enzyme, but did not report phenotypic effects on growth (Temple et al., 1993, Molecular and General Genetics 236: 315-325). Corruzi et al. have reported that transgenic tobacco overexpressing a pea cytosolic GS1 transgene under the control of the CaMV S35 promoter show increased GS activity, increased cytosolic GS protein, and improved growth characteristics (U.S. Pat. No. 6,107,547). Unkefer et al. have more recently reported that transgenic tobacco plants overexpressing the Alfalfa GS1 in foliar tissues, which had been screened for increased leaf-to-root GS activity following genetic segregation by selfing to achieve increased GS1 transgene copy number, were found to produce increased 2-hydroxy-5-oxoproline levels in their foliar portions, which was found to lead to markedly increased growth rates over wildtype tobacco plants (see, U.S. Pat. Nos. 6,555,500; 6,593,275; and 6,831,040).

Unkefer et al. have further described the use of 2-hydroxy-5-oxoproline (also known as 2-oxoglutaramate) to improve plant growth (U.S. Pat. Nos. 6,555,500; 6,593,275; 6,831,040). In particular, Unkefer et al. disclose that increased concentrations of 2-hydroxy-5-oxoproline in foliar tissues (relative to root tissues) triggers a cascade of events that result in increased plant growth characteristics. Unkefer et al. describe methods by which the foliar concentration of 2-hydroxy-5-oxoproline may be increased in order to trigger increased plant growth characteristics, specifically, by applying a solution of 2-hydroxy-5-oxoproline directly to the foliar portions of the plant and over-expressing glutamine synthetase preferentially in leaf tissues.

A number of transaminase and hydrolyase enzymes known to be involved in the synthesis of 2-hydroxy-5-oxoproline in animals have been identified in animal liver and kidney tissues (Cooper and Meister, 1977, CRC Critical Reviews in Biochemistry, pages 281-303; Meister, 1952, J. Biochem. 197: 304). In plants, the biochemical synthesis of 2-hydroxy-5-oxoproline has been known but has been poorly characterized. Moreover, the function of 2-hydroxy-5-oxoproline in plants and the significance of its pool size (tissue concentration) are unknown. Finally, the art provides no specific guidance as to precisely what transaminase(s) or hydrolase(s) may exist and/or be active in catalyzing the synthesis of 2-hydroxy-5-oxoproline in plants, and no such plant transaminases have been reported, isolated or characterized.

### SUMMARY OF THE INVENTION

The invention relates to transgenic plants exhibiting dramatically enhanced growth rates, greater seed and fruit/pod yields, earlier and more productive flowering, more efficient nitrogen utilization, increased tolerance to high salt conditions, and increased biomass yields. In one embodiment, transgenic plants engineered to over-express both glutamine phenylpyruvate transaminase (GPT) and glutamine synthetase (GS) are provided. The GPT+GS double-transgenic plants of the invention consistently exhibit enhanced growth characteristics, with TO generation lines showing an increase in biomass over wild type counterparts of between 50% and 300%. Generations that result from sexual crosses and/or selfing typically perform even better, with some of the double-transgenic plants achieving an astounding four-fold biomass increase over wild type plants. Similarly, flower and fruit or pod yields are also tremendously improved, with TO generation lines typically showing 50% to 70% increases over their wild type counterparts, and in some cases showing a 100% increase. Transgenic plants exhibiting such enhanced growth phenotypic characteristics have been successfully generated across a spectrum of individual plant species, using various transformation methodologies, different expression vectors and promoters, and heterologous and homologous transgene sequences from a variety of species, as exemplified by the numerous working examples provided herein. This invention, therefore, provides a fundamental break-though technology that has the potential to transform virtually all areas of agriculture.

Applicants have identified the enzyme glutamine phenylpyruvate transaminase (GPT) as a catalyst of 2-hydroxy-5-oxoproline (2-oxoglutaramate) synthesis in plants. 2-oxoglutaramate is a powerful signal metabolite which regulates the function of a large number of genes involved in the photosynthesis apparatus, carbon fixation and nitrogen metabolism. The invention provides isolated nucleic acid molecules encoding GPT, and discloses the novel finding that the encoded enzyme is directly involved in the synthesis of 2-hydroxy-5-oxoproline. This aspect of the invention is exemplified herein by the disclosure of GPT polynucleotides encoding GPTs from several species, including Arabidopsis, Grape, Rice, Soybean, Barley, Bamboo and a non-plant homolog from Zebra fish, most of which have been expressed as recombinant GPTs and confirmed as having GPT activity.

The invention further provides transgenic plants which express both a GPT transgene and a GS transgene. The expression of these two transgenes in such “double-transgene” plants results in a substantially increased rate of carbon dioxide fixation and an extremely potent growth enhancing effect, as these plants exhibit very significantly and sometimes tremendously enhanced growth rates and flower/fruit/pod/seed yields. Methods for the generation of such growth-enhanced transgenic plants are provided.

By preferentially increasing the concentration of the signal metabolite 2-oxoglutaramate (i.e., in foliar tissues), the transgenic plants of the invention are capable of producing higher overall yields over shorter periods of time, and therefore may provide agricultural industries with enhanced productivity across a wide range of crops. Importantly, unlike many transgenic plants described to date, the invention utilizes natural plant genes encoding a natural plant enzyme. The enhanced growth characteristics of the transgenic plants of the invention is achieved essentially by introducing additional GPT and GS capacity into the plant. Thus, the transgenic plants of the invention do not express any toxic substances, growth hormones, viral or bacterial gene products, and are therefore free of many of the concerns that have heretofore impeded the adoption of transgenic plants in certain parts of the World.

In one embodiment, the invention provides a transgenic plant comprising a GPT transgene and a GS transgene, wherein said GPT transgene and said GS transgene are operably linked to a plant promoter. In a specific embodiment, the GS transgene is a GS1 transgene. In another specific embodiment, the GPT transgene encodes a polypeptide having an amino acid sequence selected from the group consisting of (a) SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO:31, SEQ ID NO: 32, SEQ ID NO: 33, SEQ ID NO: 34, SEQ ID NO: 35 and SEQ ID NO: 36, and (b) an amino acid sequence that is at least 75% identical to any one of SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO: 31, SEQ ID NO: 32, SEQ ID NO: 33, SEQ ID NO: 34, SEQ ID NO: 35 and SEQ ID NO: 36 and has GPT activity. In yet another specific embodiment, the GS transgene encodes a polypeptide having an amino acid sequence selected from the group consisting of (a) SEQ ID NO: 4 and SEQ ID NO: 7 from residue 11, and (b) an amino acid sequence that is at least 75% identical to SEQ ID NO: 4 or SEQ ID NO: 7. In some embodiments, the GPT and GS transgenes are incorporated into the genome of the plant. The transgenic plant of the invention may be a monocotyledonous or a dicotyledonous plant.

The invention also provides progeny of any generation of the transgenic plants of the invention, wherein said progeny comprises a GPT transgene and a GS transgene, as well as a seed of any generation of the transgenic plants of the invention, wherein said seed comprises said GPT transgene and said GS transgene. The transgenic plants of the invention may display one or more enhanced growth characteristics rate when compared to an analogous wild-type or untransformed plant, including without limitation increased growth rate, biomass yield, seed yield, flower or flower bud yield, fruit or pod yield, larger leaves, and may also display increased levels of GPT and/or GS activity, and/or increased levels of 2-oxoglutaramate. In some embodiments, the transgenic plants of the invention display increased nitrogen use efficiency or increased tolerance to salt or saline conditions.

Methods for producing the transgenic plants of the invention and seeds thereof are also provided, including methods for producing a plant having enhanced growth properties, increased nitrogen use efficiency and increased tolerance to germination or growth in salt or saline conditions, relative to an analogous wild type or untransformed plant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Nitrogen assimilation and 2-oxoglutaramate biosynthesis: schematic of metabolic pathway.

FIG. 2. Photograph showing comparison of transgenic tobacco plants over-expressing either GS1 or GPT, compared to wild type tobacco plant. From left to right: wild type plant, Alfalfa GS1 transgene, Arabidopsis GPT transgene. See Examples 3 and 5, infra.

FIG. 3. Photograph showing comparison of transgenic Micro-Tom tomato plants over-expressing either GS1 or GPT, compared to wild type tomato plant. From left to right: wild type plant, Alfalfa GS1 transgene, Arabidopsis GPT transgene. See Examples 4 and 6, infra.

FIG. 4. Photographs showing comparisons of leaf sizes between wild type and GS1 or GPT transgenic tobacco plants. A: Comparison between leaves from GS1 transgenic tobacco (bottom leaf) and wild type (top leaf). B: Comparison between leaves from GPT transgenic tobacco (bottom leaf) and wild type (top leaf).

FIG. 5. Photographs showing comparisons of transgenic tobacco plants generated from various crosses between GS1 and GPT transgenic tobacco lines with wild type and single transgene plants. A-C: Cross 2, 3 and 7, respectively. See Example 7, infra.

FIG. 6. Photographs showing comparisons of leaf sizes between wild type and crosses between GS1 and GPT transgenic tobacco plants. A: Comparison between leaves from GSXGPT Cross 3 (bottom leaf) and wild type (top leaf). B: Comparison between leaves from GSXGPT Cross 7 (bottom leaf) and wild type (top leaf). See Example 7, infra.

FIG. 7. Photograph of transgenic pepper plant (right) and wild type control pepper plant (left), showing larger pepper fruit yield in the transgenic plant relative to the wild type control plant. See Example 8, infra.

FIG. 8. Transgenic bean plants compared to wild type control bean plants (several transgenic lines expressing Arabidopsis GPT and GS transgenes). Upper Left: plant heights on various days; Upper right: flower bud numbers; Lower left: flower numbers; Lower right: bean pod numbers. Wildtype is the control, and lines 2A, 4A and 5B are all transgenic plant lines. See Example 9, infra.

FIG. 9. Photograph of transgenic bean plant (right) and wild type control bean plant (left), showing increased growth in the transgenic plant relative to the wild type control plant. Transgenic line expressing Arabidopsis GPT and GS transgenes. See Example 9, infra.

FIG. 10. Transgenic bean plants pods, flowers and flower buds compared to wild type control bean plants (transgenic line expressing grape GPT and Arabidopsis GS transgenes). See Example 10, infra.

FIG. 11. Photograph of transgenic bean plant (right) and wild type control bean plant (left), showing increased growth in the transgenic plant relative to the wild type control plant. Transgenic line expressing Grape GPT and Arabidopsis GS transgenes. See Example 10, infra.

FIG. 12. Transgenic Cowpea Line A plants compared to wild type control Cowpea plants (transgenic line expressing Arabidopsis GPT and GS transgenes), showing that the transgenic plants grow faster and flower and set pods sooner than wild type control plants. (A) Relative height and longest leaf measurements as of May 21, (B) Relative trifolate leafs and flower buds as of June 18, (C) Relative numbers of flowers, flower buds and pea pods as of June 22. See Example 11, infra.

FIG. 13. Photograph of transgenic Cowpea Line A plant (right) and wild type control Cowpea plant (left), showing increased growth in the transgenic plant relative to the wild type control plant. Transgenic line expressing Arabidopsis GPT and GS transgenes. See Example 11, infra.

FIG. 14. Transgenic Cowpea Line G plants compared to wild type control Cowpea plants (transgenic line expressing Grape GPT and Arabidopsis GS transgenes), showing that the transgenic plants grow faster and flower and set pods sooner than wild type control plants. (A) plant heights, (B) flowers and pea pod numbers, (C) leaf bud and trifolate numbers. See Example 12, infra.

FIG. 15. Photograph of transgenic Cowpea Line G plant (right) and wild type control Cowpea plant (left), showing increased growth in the transgenic plant relative to the wild type control plant. Transgenic line expressing Grape GPT and Arabidopsis GS transgenes. See Example 12, infra.

FIG. 16. Photograph of transgenic Cantaloupe plant (right) and wild type control Cantaloupe plant (left), showing increased growth in the transgenic plant relative to the wild type control plant. Transgenic line expressing Arabidopsis GPT and GS transgenes. See Example 14, infra.

FIG. 17. Photograph of transgenic Pumpkin plants (right) and wild type control Pumpkin plants (left), showing increased growth in the transgenic plants relative to the wild type control plants. Transgenic lines expressing Arabidopsis GPT and GS transgenes. See Example 15, infra.

FIG. 18. Photograph of transgenic Arabidopsis plants (right) and wild type control Arabidopsis plants (left), showing increased growth in the transgenic plants relative to the wild type control plants. Transgenic lines expressing Arabidopsis GPT and GS transgenes. See Example 16, infra.

FIG. 19. Transgenic tomato plants expressing Arabidopsis GPT and GS transgenes compared to control tomato plants. (A) Photograph of transgenic tomato plant leaves (right) vs. wild type control leaves (left) showing larger leaves in the transgenic plant. (B) Photograph of transgenic tomato plants (right) and wild type control plants (left), showing increased growth in the transgenic plants relative to the wild type control plants. See Example 17, infra.

FIG. 20. Photograph of transgenic Camelina plant (right) and wild type control Camelina plant (left), showing increased growth in the transgenic plant relative to the wild type control plant. Transgenic line expressing Arabidopsis GPT and GS transgenes. See Example 18, infra.

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Penak, ed., Humana Press, 1^{st} edition, 2004); and, Agrobacterium Protocols (Wan, ed., Humana Press, 2^{nd} edition, 2006). As appropriate, procedures involving the use of commercially available kits and reagents are generally carried out in accordance with manufacturer defined protocols and/or parameters unless otherwise noted.

The term “nucleic acid” refers to deoxyribonucleotides or ribonucleotides and polymers thereof (“polynucleotides”) in either single- or double-stranded form. Unless specifically limited, the term “polynucleotide” encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

The term “promoter” refers to anucleic acid control sequence or sequences that direct transcription of an operably linked nucleic acid. As used herein, a “plant promoter” is a promoter that functions in plants. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “plant” includes whole plants, plant organs (e.g., leaves, stems, flowers, roots, reproductive organs, embryos and parts thereof, etc.), seedlings, seeds and plant cells and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous.

The terms “GPT polynucleotide” and “GPT nucleic acid” are used interchangeably herein, and refer to a full length or partial length polynucleotide sequence of a gene which encodes a polypeptide involved in catalyzing the synthesis of 2-oxoglutaramate, and includes polynucleotides containing both translated (coding) and un-translated sequences, as well as the complements thereof. The term “GPT coding sequence” refers to the part of the gene which is transcribed and encodes a GPT protein. The term “targeting sequence” refers to the amino terminal part of a protein which directs the protein into a subcellular compartment of a cell, such as a chloroplast in a plant cell. GPT polynucleotides are further defined by their ability to hybridize under defined conditions to the GPT polynucleotides specifically disclosed herein, or to PCR products derived therefrom.

A “GPT transgene” is a nucleic acid molecule comprising a GPT polynucleotide which is exogenous to transgenic plant, or plant embryo, organ or seed, harboring the nucleic acid molecule, or which is exogenous to an ancestor plant, or plant embryo, organ or seed thereof, of a transgenic plant harboring the GPT polynucleotide.

The terms “GS polynucleotide” and “GS nucleic acid” are used interchangeably herein, and refer to a full length or partial length polynucleotide sequence of a gene which encodes a glutamine synthetase protein, and includes polynucleotides containing both translated (coding) and un-translated sequences, as well as the complements thereof. The term “GS coding sequence” refers to the part of the gene which is transcribed and encodes a GS protein. The terms “GS1 polynucleotide” and “GS1 nucleic acid” are used interchangeably herein, and refer to a full length or partial length polynucleotide sequence of a gene which encodes a glutamine synthetase isoform 1 protein, and includes polynucleotides containing both translated (coding) and un-translated sequences, as well as the complements thereof. The term “GS1 coding sequence” refers to the part of the gene which is transcribed and encodes a GS1 protein.

A “GS transgene” is a nucleic acid molecule comprising a GS polynucleotide which is exogenous to transgenic plant, or plant embryo, organ or seed, harboring the nucleic acid molecule, or which is exogenous to an ancestor plant, or plant embryo, organ or seed thereof, of a transgenic plant harboring the GS polynucleotide. A “GS1 transgene” is a nucleic acid molecule comprising a GS1 polynucleotide which is exogenous to transgenic plant, or plant embryo, organ or seed, harboring the nucleic acid molecule, or which is exogenous to an ancestor plant, or plant embryo, organ or seed thereof, of a transgenic plant harboring the GS1 polynucleotide.

Exemplary GPT polynucleotides of the invention are presented herein, and include GPT coding sequences for Arabidopsis, Rice, Barley, Bamboo, Soybean, Grape, and Zebra Fish GPTs.

Partial length GPT polynucleotides include polynucleotide sequences encoding N- or C-terminal truncations of GPT, mature GPT (without targeting sequence) as well as sequences encoding domains of GPT. Exemplary GPT polynucleotides encoding N-terminal truncations of GPT include Arabidopsis −30, −45 and −56 constructs, in which coding sequences for the first 30, 45, and 56, respectively, amino acids of the full length GPT structure of SEQ ID NO: 2 are eliminated.

In employing the GPT polynucleotides of the invention in the generation of transformed cells and transgenic plants, one of skill will recognize that the inserted polynucleotide sequence need not be identical, but may be only “substantially identical” to a sequence of the gene from which it was derived, as further defined below. The term “GPT polynucleotide” specifically encompasses such substantially identical variants. Similarly, one of skill will recognize that because of codon degeneracy, a number of polynucleotide sequences will encode the same polypeptide, and all such polynucleotide sequences are meant to be included in the term GPT polynucleotide. In addition, the term specifically includes those sequences substantially identical (determined as described below) with an GPT polynucleotide sequence disclosed herein and that encode polypeptides that are either mutants of wild type GPT polypeptides or retain the function of the GPT polypeptide (e.g., resulting from conservative substitutions of amino acids in a GPT polypeptide). The term “GPT polynucleotide” therefore also includes such substantially identical variants.

The term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “isolated” refers to material which is substantially or essentially free from components which normally accompany the material as it is found in its native or natural state. However, the term “isolated” is not intended refer to the components present in an electrophoretic gel or other separation medium. An isolated component is free from such separation media and in a form ready for use in another application or already in use in the new application/milieu. An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “heterologous” when used with reference to portions of a nucleic acid indicates that the nucleic acid comprises two or more subsequences that are not found in the same relationship to each other in nature. For instance, a nucleic acid is typically recombinantly produced, having two or more sequences from unrelated genes arranged to make a new functional nucleic acid, e.g., a nucleic acid encoding a protein from one source and a nucleic acid encoding a peptide sequence from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection. This definition also refers to the complement of a test sequence, which has substantial sequence or subsequence complementarity when the test sequence has substantial identity to a reference sequence. This definition also refers to the complement of a test sequence, which has substantial sequence or subsequence complementarity when the test sequence has substantial identity to a reference sequence.

When percentage of sequence identity is used in reference to polypeptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the polypeptide. Where sequences differ in conservative substitutions, the percent sequence identity may be adjusted upwards to correct for the conservative nature of the substitution.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. BLAST and BLAST 2.0 are used, typically with the default parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tm) for the specific sequence at a defined ionic strength pH. Low stringency conditions are generally selected to be about 15-30° C. below the Tm. Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

Genomic DNA or cDNA comprising GPT polynucleotides may be identified in standard Southern blots under stringent conditions using the GPT polynucleotide sequences disclosed here. For this purpose, suitable stringent conditions for such hybridizations are those which include a hybridization in a buffer of 40% formamide, 1M NaCl, 1% SDS at 37° C., and at least one wash in 0.2×SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions may be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

### Transgenic Plants:

The invention provides novel transgenic plants exhibiting substantially enhanced agronomic characteristics, including faster growth, greater mature plant fresh weight and total biomass, earlier and more abundant flowering, and greater fruit, pod and seed yields. The transgenic plants of the invention are generated by introducing into a plant one or more expressible genetic constructs capable of driving the expression of one or more polynucleotides encoding glutamine synthetase (GS) and glutamine phenylpyruvate transaminase (GPT). In an exemplary embodiment, single-transgene parental lines carrying either a GPT or GS1 transgene coding sequence are generated, preferably selfed until homozygous for the transgene, then crossed to generate progeny plants containing both transgenes.

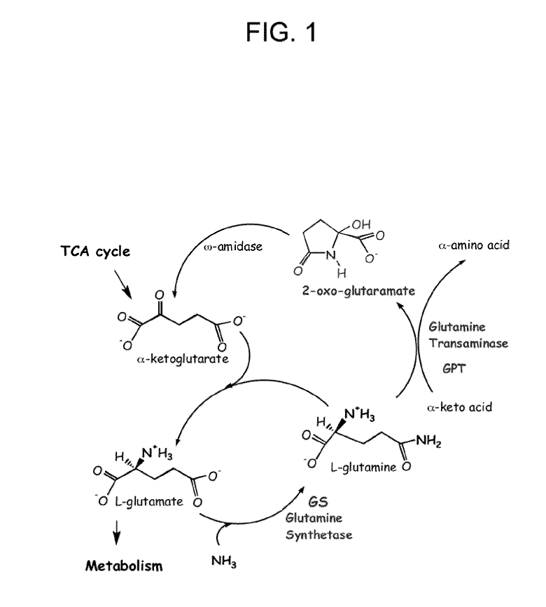

In stable transformation embodiments of the invention, one or more copies of the expressible genetic construct become integrated into the host plant genome, thereby providing increased GS and GPT enzyme capacity into the plant, which serves to mediate increased synthesis of 2-oxoglutaramate, which in turn signals metabolic gene expression, resulting in increased plant growth and the enhancement other agronomic characteristics. 2-oxoglutaramate is a metabolite which is an extremely potent effector of gene expression, metabolism and plant growth (U.S. Pat. No. 6,555,500), and which may play a pivotal role in the coordination of the carbon and nitrogen metabolism systems (Lancien et al., 2000, Enzyme Redundancy and the Importance of 2-Oxoglutarate in Higher Plants Ammonium Assimilation, Plant Physiol. 123: 817-824). See, also, the schematic of the 2-oxoglutaramate pathway shown in FIG. 1.

In one aspect of the invention, applicants have isolated a nucleic acid molecule encoding the Arabidopsis glutamine phenylpyruvate transaminase (GPT) enzyme (see Example 1, infra), and have demonstrated for the first time that the expressed recombinant enzyme is active and capable of catalyzing the synthesis of the signal metabolite, 2-oxoglutaramate (Example 2, infra). Further, applicants have demonstrated for the first time that over-expression of the Arabidopsis glutamine transaminase gene in a transformed heterologous plant results in enhanced CO₂ fixation rates and increased growth characteristics (Example 3, infra).

Applicants' previous work demonstrated that over-expression of Alfalfa GS1 gene under the control of a strong constitutive promoter results in transgenic tobacco plants with higher levels of GS activity in the leaves. These plants outgrow their wild-type counterparts, fix CO₂ faster, contain increased concentrations of total protein, as well as increased concentrations of glutamine and 2-oxoglutaramate, and show increased rates of uptake of nitrate through their roots.

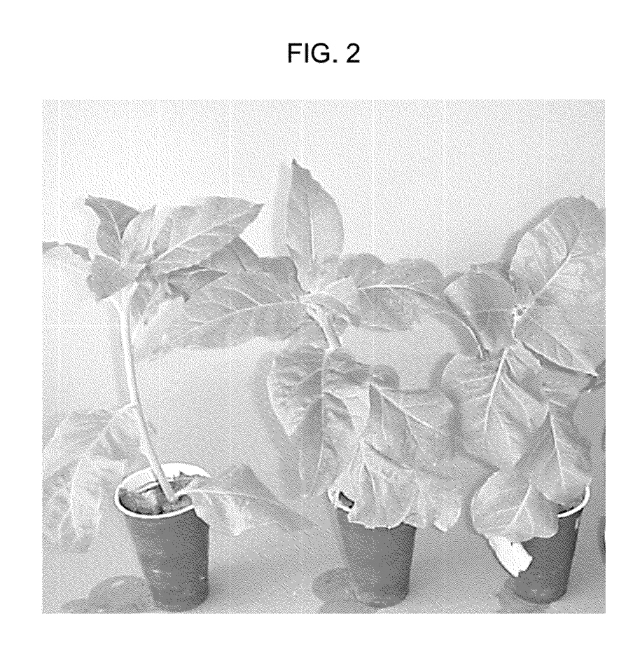

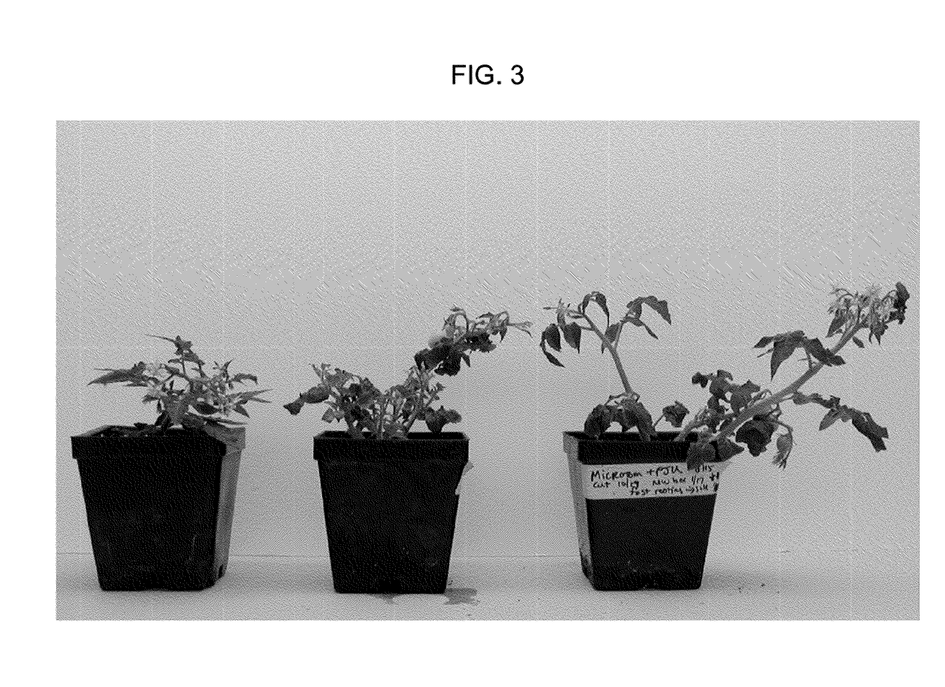

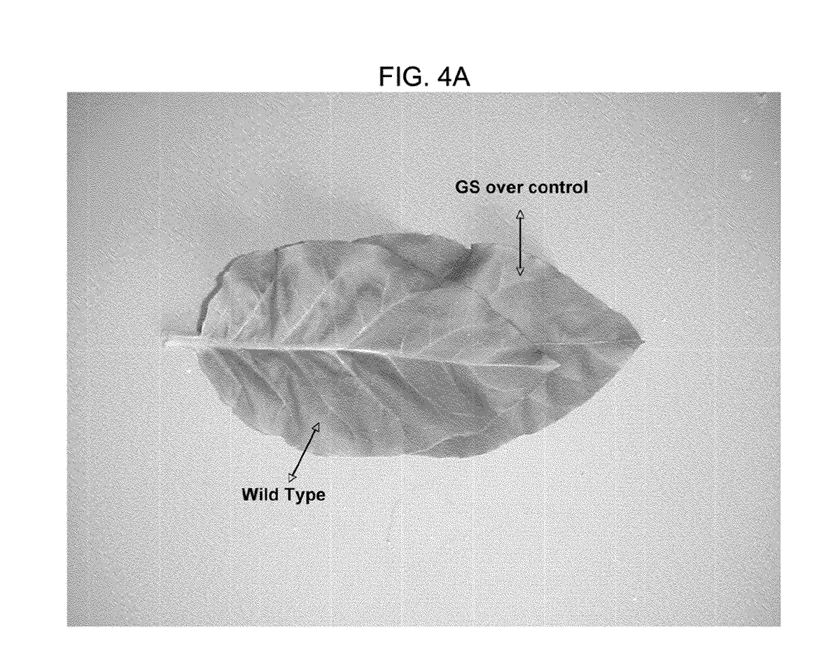

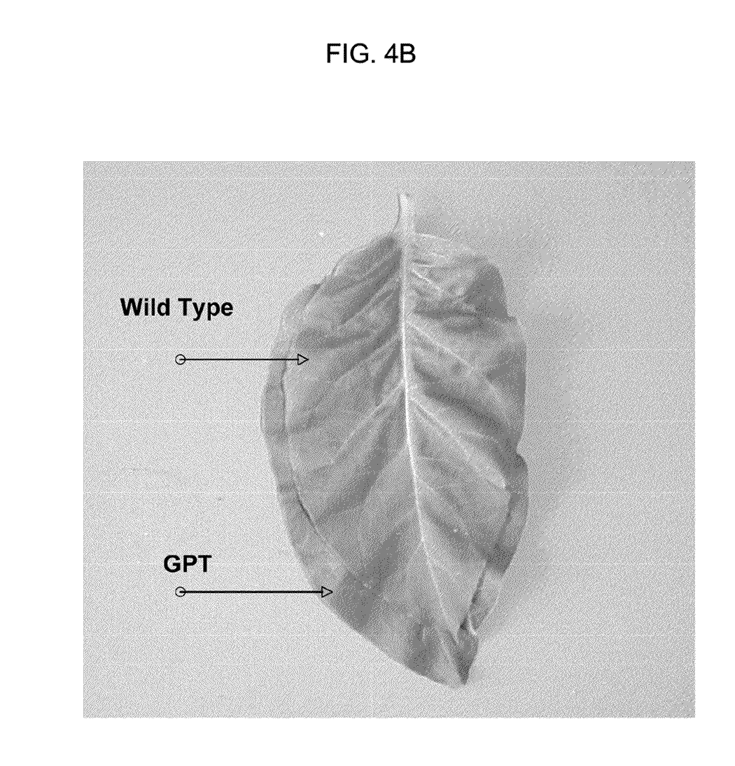

As disclosed herein (see Example 3, infra), over-expression of a transgene comprising the full-length Arabidopsis GPT coding sequence in transgenic tobacco plants also results in faster CO₂ fixation, and increased levels of total protein, glutamine and 2-oxoglutaramate. These transgenic plants also grow faster than wild-type plants (FIG. 2). Similarly, in preliminary studies conducted with tomato plants (see Example 4, infra), tomato plants transformed with the Arabidopsis GPT transgene showed significant enhancement of growth rate, flowering, and seed yield in relation to wild type control plants (FIG. 3 and Example 4, infra).

In one particular embodiment, exemplified herein by way of Examples 3, 5 and 7, infra, a first set of parental single-transgene tobacco plant lines carrying the Alfalfa GS1 gene, including 5′ and 3′ untranslated regions, were generated using Agrobacterium mediated gene transformation, under selective pressure, together with screening for the fastest growing phenotype, and selfing to transgene/phenotype homozygosity (see Example 5, infra). A second set of parental single-transgene tobacco plant lines carrying the full length coding sequence of Arabidopsis GPT were generated in the same manner (Example 3, infra). High growth rate performing plants from each of the parental lines were then sexually crossed to yield progeny lines (Example 7, infra).

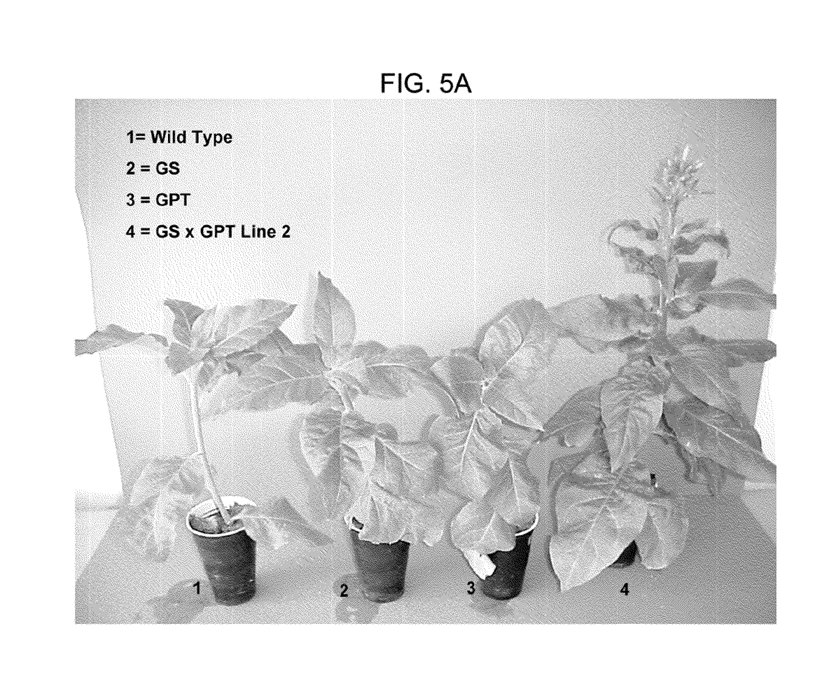

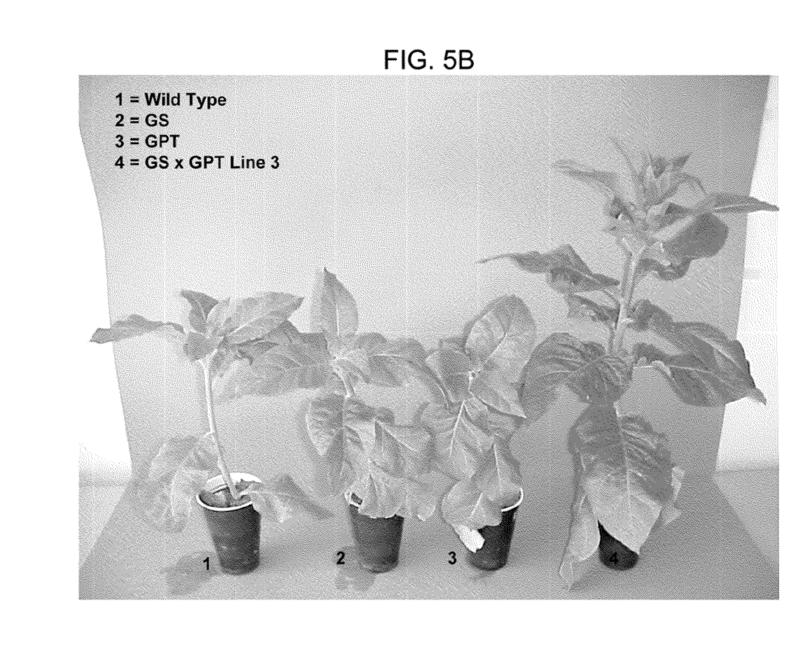

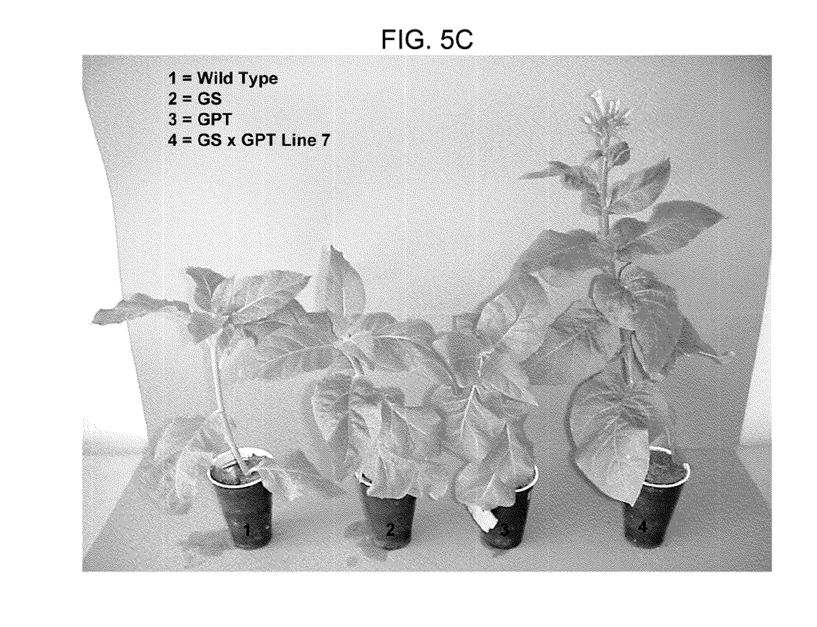

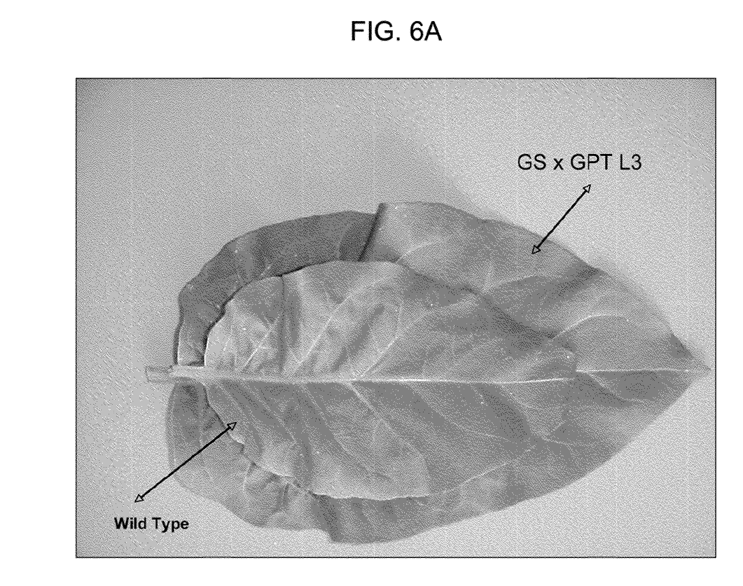

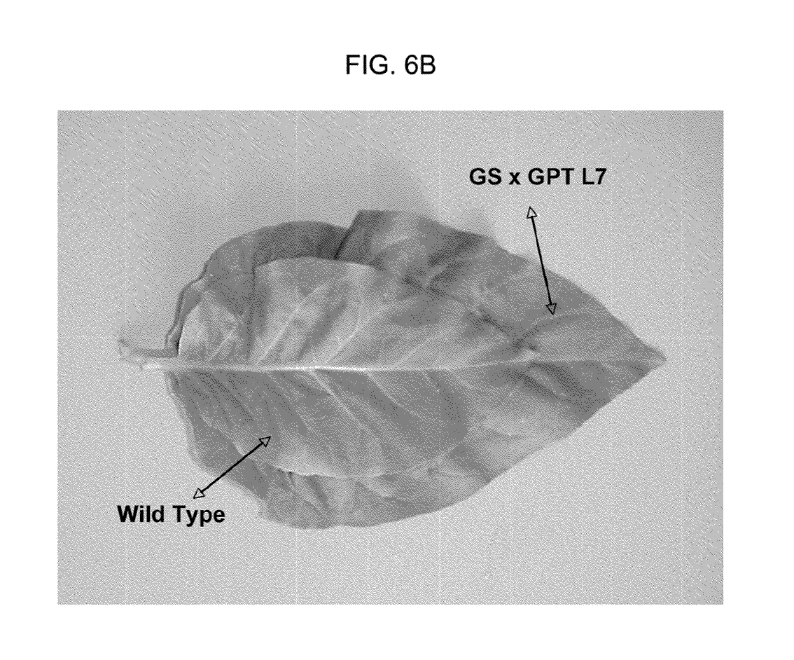

The resulting progeny from multiple crosses of Arabidopsis GS1 and GPT transgenic tobacco plants produce far better and quite surprising increases in growth rates over the single-transgene parental lines as well as wildtype plants. FIG. 5 shows photographs of double-transgene progeny from single-transgene GS1×GPT plant crosses, relative to wild type and single-transgene parental plants. FIG. 6 shows photographs comparing leaf sizes of double-transgene progeny and wild type plants. Experimentally observed growth rates in these double transgenic plants ranged between 200% and 300% over wild-type plants (Example 7, infra). Moreover, total biomass levels increased substantially in the double-transgene plants, with whole plant fresh weights typically being about two to three times the wild-type plant weights. Similarly, seed yields showed similar increases in the double-transgene plants, with seed pod production typically two to three times the wild type average, and overall seed yields exceeding wild-type plant yields by 300-400%.

In addition to the transgenic tobacco plants referenced above, various other species of transgenic plants comprising GPT and GS transgenes are specifically exemplified herein. As exemplified herein, transgenic plants showing enhanced growth characteristics have been generated in two species of Tomato (see Examples 4 and 17), Pepper (Example 8), Beans (Examples 9 and 10), Cowpea (Examples 11 and 12), Alfalfa (Example 13), Cantaloupe (Example 14), Pumpkin (Example 15), Arabidopsis (Example 16) and Camilena (Example 18). These transgenic plants of the invention were generated using a variety of transformation methodologies, including Agrobacterium-mediated callus, floral dip, seed inoculation, pod inoculation, and direct flower inoculation, as well as combinations thereof, and via sexual crosses of single transgene plants, as exemplified herein. Different GPT and GS transgenes were successfully employed in generating the transgenic plants of the invention, as exemplified herein.

The invention also provides methods of generating a transgenic plant having enhanced growth and other agronomic characteristics. In one embodiment, a method of generating a transgenic plant having enhanced growth and other agronomic characteristics comprises introducing into a plant cell an expression cassette comprising a nucleic acid molecule encoding a GPT transgene, under the control of a suitable promoter capable of driving the expression of the transgene, so as to yield a transformed plant cell, and obtaining a transgenic plant which expresses the encoded GPT. In another embodiment, a method of generating a transgenic plant having enhanced growth and other agronomic characteristics comprises introducing into a plant cell one or more nucleic acid constructs or expression cassettes comprising nucleic acid molecules encoding a GPT transgene and an GS transgene, under the control of one or more suitable promoters (and, optionally, other regulatory elements) capable of driving the expression of the transgenes, so as to yield a plant cell transformed thereby, and obtaining a transgenic plant which expresses the GPT and GS transgenes.

Based on the results disclosed herein, it is clear that any number of GPT and GS polynucleotides may be used to generate the transgenic plants of the invention. Both GS1 and GPT proteins are highly conserved among various plant species, and it is evident from the experimental data disclosed herein that closely-related non-plant GPTs may be used as well (e.g., Danio rerio GPT). With respect to GPT, numerous GPT polynucleotides derived from different species have been shown to be active and useful as GPT transgenes. Similarly, different GS polynucleotides may be used, including without limitation any plant GS1 encoding polynucleotide that generates GS activity in a host cell transformed with an expressible GS1 construct.

In a specific embodiment, the GPT transgene is a GPT polynucleotide encoding an Arabidopsis derived GPT, such as the GPT of SEQ ID NO: 2, SEQ ID NO: 21 and SEQ ID NO: 30, and the GS transgene is a GS polynucleotide encoding an Alfalfa derived GS1 (i.e., SEQ ID NO: 4) or an Arabidopsis derived GS1 (SEQ ID NO: 7). The GPT transgene may be encoded by the nucleotide sequence of SEQ ID NO: 1; a nucleotide sequence having at least 75% and more preferably at least 80% identity to SEQ ID NO: 1, and encoding a polypeptide having GPT activity; a nucleotide sequence encoding the polypeptide of SEQ ID NO: 2, or a polypeptide having at least 75% and more preferably at least 80% sequence identity thereto which has GPT activity; and a nucleotide sequence encoding the polypeptide of SEQ ID NO: 2 truncated at its amino terminus by between 30 to 56 amino acid residues, or a polypeptide having at least 75% and more preferably at least 80% sequence identity thereto which has GPT activity. The GS1 transgene may be encoded by the polynucleotide of SEQ ID NO: 3 or SEQ ID NO: 6 or a nucleotide sequence having at least 75% and more preferably at least 80% identity to SEQ ID NO: 3 or SEQ ID NO: 6, and encoding a polypeptide having GPT activity; and a nucleotide sequence encoding the polypeptide of SEQ ID NO: 4 or 7, or a polypeptide having at least 75% and more preferably at least 80% sequence identity thereto which has GS activity.

In another specific embodiment, the GPT transgene is a GPT polynucleotide encoding a Grape derived GPT, such as the Grape GPTs of SEQ ID NO: 9 and SEQ ID NO: 31, and the GS transgene is a GS1 polynucleotide. The GPT transgene may be encoded by the nucleotide sequence of SEQ ID NO: 8; a nucleotide sequence having at least 75% and more preferably at least 80% identity to SEQ ID NO: 8, and encoding a polypeptide having GPT activity; a nucleotide sequence encoding the polypeptide of SEQ ID NO: 9 or SEQ ID NO: 31, or a polypeptide having at least 75% and more preferably at least 80% sequence identity thereto which has GPT activity.

In yet another specific embodiment, the GPT transgene is a GPT polynucleotide encoding a Rice derived GPT, such as the Rice GPTs of SEQ ID NO: 11 and SEQ ID NO: 32, and the GS transgene is a GS1 polynucleotide. The GPT transgene may be encoded by the nucleotide sequence of SEQ ID NO: 10; a nucleotide sequence having at least 75% and more preferably at least 80% identity to SEQ ID NO: 10, and encoding a polypeptide having GPT activity; a nucleotide sequence encoding the polypeptide of SEQ ID NO: 11 or SEQ ID NO: 32, or a polypeptide having at least 75% and more preferably at least 80% sequence identity thereto which has GPT activity.

In yet another specific embodiment, the GPT transgene is a GPT polynucleotide encoding a Soybean derived GPT, such as the Soybean GPTs of SEQ ID NO: 13, SEQ ID NO: 33 or SEQ ID NO: 33 with a further Isoleucine at the N-terminus of the sequence, and the GS transgene is a GS1 polynucleotide. The GPT transgene may be encoded by the nucleotide sequence of SEQ ID NO: 12; a nucleotide sequence having at least 75% and more preferably at least 80% identity to SEQ ID NO: 12, and encoding a polypeptide having GPT activity; a nucleotide sequence encoding the polypeptide of SEQ ID NO: 13 or SEQ ID NO: 33 or SEQ ID NO: 33 with a further Isoleucine at the N-terminus of the sequence, or a polypeptide having at least 75% and more preferably at least 80% sequence identity thereto which has GPT activity.

In yet another specific embodiment, the GPT transgene is a GPT polynucleotide encoding a Barley derived GPT, such as the Barley GPTs of SEQ ID NO: 15 and SEQ ID NO: 34, and the GS transgene is a GS1 polynucleotide. The GPT transgene may be encoded by the nucleotide sequence of SEQ ID NO: 14; a nucleotide sequence having at least 75% and more preferably at least 80% identity to SEQ ID NO: 10, and encoding a polypeptide having GPT activity; a nucleotide sequence encoding the polypeptide of SEQ ID NO: 15 or SEQ ID NO: 34, or a polypeptide having at least 75% and more preferably at least 80% sequence identity thereto which has GPT activity.

In yet another specific embodiment, the GPT transgene is a GPT polynucleotide encoding a Zebra fish derived GPT, such as the Zebra fish GPTs of SEQ ID NO: 17 and SEQ ID NO: 35, and the GS transgene is a GS1 polynucleotide. The GPT transgene may be encoded by the nucleotide sequence of SEQ ID NO: 16; a nucleotide sequence having at least 75% and more preferably at least 80% identity to SEQ ID NO: 16, and encoding a polypeptide having GPT activity; a nucleotide sequence encoding the polypeptide of SEQ ID NO: 17 or SEQ ID NO: 35, or a polypeptide having at least 75% and more preferably at least 80% sequence identity thereto which has GPT activity.

In yet another specific embodiment, the GPT transgene is a GPT polynucleotide encoding a Bamboo derived GPT, such as the Bamboo GPT of SEQ ID NO: 36, and the GS transgene is a GS1 polynucleotide. The GPT transgene may be encoded by a nucleotide sequence encoding the polypeptide of SEQ ID NO: 36, or a polypeptide having at least 75% and more preferably at least 80% sequence identity thereto which has GPT activity.

Other GPT polynucleotides suitable for use as GPT transgenes in the practice of the invention may be obtained by various means, as will be appreciated by one skilled in the art, tested for the ability to direct the expression of a GPT with GPT activity in a recombinant expression system (i.e., E. coli (see Examples 20-23), in a transient in planta expression system (see Example 19), or in a transgenic plant (see Examples 1-18).

### Transgene Constructs/Expression Vectors

In order to generate the transgenic plants of the invention, the gene coding sequence for the desired transgene(s) must be incorporated into a nucleic acid construct (also interchangeably referred to herein as a/an (transgene) expression vector, expression cassette, expression construct or expressible genetic construct), which can direct the expression of the transgene sequence in transformed plant cells. Such nucleic acid constructs carrying the transgene(s) of interest may be introduced into a plant cell or cells using a number of methods known in the art, including but not limited to electroporation, DNA bombardment or biolistic approaches, microinjection, and via the use of various DNA-based vectors such as Agrobacterium tumefaciens and Agrobacterium rhizogenes vectors. Once introduced into the transformed plant cell, the nucleic acid construct may direct the expression of the incorporated transgene(s) (i.e., GPT), either in a transient or stable fashion. Stable expression is preferred, and is achieved by utilizing plant transformation vectors which are able to direct the chromosomal integration of the transgene construct. Once a plant cell has been successfully transformed, it may be cultivated to regenerate a transgenic plant.

A large number of expression vectors suitable for driving the constitutive or induced expression of inserted genes in transformed plants are known. In addition, various transient expression vectors and systems are known. To a large extent, appropriate expression vectors are selected for use in a particular method of gene transformation (see, infra). Broadly speaking, a typical plant expression vector for generating transgenic plants will comprise the transgene of interest under the expression regulatory control of a promoter, a selectable marker for assisting in the selection of transformants, and a transcriptional terminator sequence.

More specifically, the basic elements of a nucleic acid construct for use in generating the transgenic plants of the invention are: a suitable promoter capable of directing the functional expression of the transgene(s) in a transformed plant cell, the transgene (s) (i.e., GPT coding sequence) operably linked to the promoter, preferably a suitable transcription termination sequence (i.e., nopaline synthetic enzyme gene terminator) operably linked to the transgene, and sometimes other elements useful for controlling the expression of the transgene, as well as one or more selectable marker genes suitable for selecting the desired transgenic product (i.e., antibiotic resistance genes).

As Agrobacterium tumefaciens is the primary transformation system used to generate transgenic plants, there are numerous vectors designed for Agrobacterium transformation. For stable transformation, Agrobacterium systems utilize “binary” vectors that permit plasmid manipulation in both E. coli and Agrobacterium, and typically contain one or more selectable markers to recover transformed plants (Hellens et al., 2000, Technical focus: A guide to Agrobacterium binary Ti vectors. Trends Plant Sci 5:446-451). Binary vectors for use in Agrobacterium transformation systems typically comprise the borders of T-DNA, multiple cloning sites, replication functions for Escherichia coli and A. tumefaciens, and selectable marker and reporter genes.

So-called “super-binary” vectors provide higher transformation efficiencies, and generally comprise additional virulence genes from a Ti (Komari et al., 2006, Methods Mol. Biol. 343: 15-41). Super binary vectors are typically used in plants which exhibit lower transformation efficiencies, such as cereals. Such additional virulence genes include without limitation virB, virE, and virG (Vain et al., 2004, The effect of additional virulence genes on transformation efficiency, transgene integration and expression in rice plants using the pGreen/pSoup dual binary vector system. Transgenic Res. 13: 593-603; Srivatanakul et al., 2000, Additional virulence genes influence transgene expression: transgene copy number, integration pattern and expression. J. Plant Physiol. 157, 685-690; Park et al., 2000, Shorter T-DNA or additional virulence genes improve Agrobacterium-mediated transformation. Theor. Appl. Genet. 101, 1015-1020; Jin et al., 1987, Genes responsible for the supervirulence phenotype of Agrobacterium tumefaciens A281. J. Bacteriol. 169: 4417-4425).

In the embodiments exemplified herein (see Examples, infra), expression vectors which place the inserted transgene(s) under the control of the constitutive CaMV 35S promoter and the RuBisCo promoter are employed. A number of expression vectors which utilize the CaMV 35S and RuBsCo promoter are known and/or commercially available and/or derivable using ordinary skill in the art. Additionally, numerous promoters suitable for directing the expression of the transgene are known and may be used in the practice of the invention, as further described, infra.

### Plant Promoters

A large number of promoters which are functional in plants are known in the art. In constructing GPT and GS transgene constructs, the selected promoter(s) may be constitutive, non-specific promoters such as the Cauliflower Mosaic Virus 35S ribosomal promoter (CaMV 35S promoter), which is widely employed for the expression of transgenes in plants. Examples of other strong constitutive promoters include without limitation the rice actin 1 promoter, the CaMV 19S promoter, the Ti plasmid nopaline synthase promoter, the alcohol dehydrogenase promoter and the sucrose synthase promoter.

Alternatively, in some embodiments, it may be desirable to select a promoter based upon the desired plant cells to be transformed by the transgene construct, the desired expression level of the transgene, the desired tissue or subcellular compartment for transgene expression, the developmental stage targeted, and the like.

For example, when expression in photosynthetic tissues and compartments is desired, a promoter of the ribulose bisphosphate carboxylase (RuBisCo) gene may be employed. In the Examples which follow, expressible nucleic acid constructs comprising GPT and GS1 transgenes under the control of a tomato RuBisCo promoter were prepared and used in the generation of transgenic plants or to assay for GPT activity in planta or in E. coli.

When the expression in seeds is desired, promoters of various seed storage protein genes may be employed. For expression in fruits, a fruit-specific promoter such as tomato 2A11 may be used. Examples of other tissue specific promoters include the promoters encoding lectin (Vodkin et al., 1983, Cell 34:1023-31; Lindstrom et al., 1990, Developmental Genetics 11:160-167), corn alcohol dehydrogenase 1 (Vogel et al, 1989, J. Cell. Biochem. (Suppl. 0) 13:Part D; Dennis et al., 1984, Nucl. Acids Res., 12(9): 3983-4000), corn light harvesting complex (Simpson, 1986, Science, 233: 34-38; Bansal et al., 1992, Proc. Natl. Acad. Sci. USA, 89: 3654-3658), corn heat shock protein (Odell et al., 1985, Nature, 313: 810-812; Rochester et al., 1986, EMBO J., 5: 451-458), pea small subunit RuBP carboxylase (Poulsen et al., 1986, Mol. Gen. Genet., 205(2): 193-200; Cashmore et al., 1983, Gen. Eng. Plants, Plenum Press, New York, pp 29-38), Ti plasmid mannopine synthase and Ti plasmid nopaline synthase (Langridge et al., 1989, Proc. Natl. Acad. Sci. USA, 86: 3219-3223), petunia chalcone isomerase (Van Tunen et al., 1988, EMBO J. 7(5): 1257-1263), bean glycine rich protein 1 (Keller et al., 1989, EMBO J. 8(5): 1309-1314), truncated CaMV 35S (Odell et al., 1985, supra), potato patatin (Wenzler et al., 1989, Plant Mol. Biol. 12: 41-50), root cell (Conkling et al., 1990, Plant Physiol. 93: 1203-1211), maize zein (Reina et al., 1990, Nucl. Acids Res. 18(21): 6426; Kriz et al., 1987, Mol. Gen. Genet. 207(1): 90-98; Wandelt and Feix, 1989, Nuc. Acids Res. 17(6): 2354; Langridge and Feix, 1983, Cell 34: 1015-1022; Reina et al., 1990, Nucl. Acids Res. 18(21): 6426), globulin-1 (Belanger and Kriz, 1991, Genetics 129: 863-872), α-tubulin (Carpenter et al., 1992, Plant Cell 4(5): 557-571; Uribe et al., 1998, Plant Mol. Biol. 37(6): 1069-1078), cab (Sullivan, et al., 1989, Mol. Gen. Genet. 215(3): 431-440), PEPCase (Hudspeth and Grula, 1989, Plant Mol. Biol. 12: 579-589), R gene complex (Chandler et al., 1989, The Plant Cell 1: 1175-1183), chalcone synthase (Franken et al., 1991, EMBO J. 10(9): 2605-2612) and glutamine synthetase promoters (U.S. Pat. No. 5,391,725; Edwards et al., 1990, Proc. Natl. Acad. Sci. USA 87: 3459-3463; Brears et al., 1991, Plant J. 1(2): 235-244).

In addition to constitutive promoters, various inducible promoter sequences may be employed in cases where it is desirable to regulate transgene expression as the transgenic plant regenerates, matures, flowers, etc. Examples of such inducible promoters include promoters of heat shock genes, protection responding genes (i.e., phenylalanine ammonia lyase; see, for example Bevan et al., 1989, EMBO J. 8(7): 899-906), wound responding genes (i.e., cell wall protein genes), chemically inducible genes (i.e., nitrate reductase, chitinase) and dark inducible genes (i.e., asparagine synthetase; see, for example U.S. Pat. No. 5,256,558). Also, a number of plant nuclear genes are activated by light, including gene families encoding the major chlorophyll a/b binding proteins (cab) as well as the small subunit of ribulose-1,5-bisphosphate carboxylase (rbcS) (see, for example, Tobin and Silverthorne, 1985, Annu. Rev. Plant Physiol. 36: 569-593; Dean et al., 1989, Annu. Rev. Plant Physiol. 40: 415-439.).

Other inducible promoters include ABA- and turgor-inducible promoters, the auxin-binding protein gene promoter (Schwob et al., 1993, Plant J. 4(3): 423-432), the UDP glucose flavonoid glycosyl-transferase gene promoter (Ralston et al., 1988, Genetics 119(1): 185-197); the MPI proteinase inhibitor promoter (Cordero et al., 1994, Plant J. 6(2): 141-150), the glyceraldehyde-3-phosphate dehydrogenase gene promoter (Kohler et al., 1995, Plant Mol. Biol. 29(6): 1293-1298; Quigley et al., 1989, J. Mol. Evol. 29(5): 412-421; Martinez et al., 1989, J. Mol. Biol. 208(4): 551-565) and light inducible plastid glutamine synthetase gene from pea (U.S. Pat. No. 5,391,725; Edwards et al., 1990, supra).

For a review of plant promoters used in plant transgenic plant technology, see Potenza et al., 2004, In Vitro Cell. Devel. Biol—Plant, 40(1): 1-22. For a review of synthetic plant promoter engineering, see, for example, Venter, M., 2007, Trends Plant Sci., 12(3): 118-124.

### Glutamine Phenylpyruvate Transaminase (GPT) Transgene

The present invention discloses for the first time that plants contain a glutamine phenylpyruvate transaminase (GPT) enzyme which is directly functional in the synthesis of the signal metabolite 2-hydroxy-5-oxoproline. Until now, no plant glutamine phenylpyruvate transaminase with a defined function has been described. Applicants have isolated and tested GPT polynucleotide coding sequences derived from several plant and animal species, and have successfully incorporated the gene into heterologous transgenic host plants which exhibit markedly improved growth characteristics, including faster growth, higher foliar protein content, increased glutamine synthetase activity in foliar tissue, and faster CO₂ fixation rates.

In the practice of the invention, the GPT gene functions as one of at least two transgenes incorporated into the transgenic plants of the invention, the other being the glutamine sythetase gene (see infra).

It is expected that all plant species contain a GPT which functions in the same metabolic pathway, involving the biosynthesis of the signal metabolite 2-hydroxy-5-oxoproline. Thus, in the practice of the invention, any plant gene encoding a GPT homolog or functional variants thereof may be useful in the generation of transgenic plants of this invention. Moreover, given the structural similarity between various plant GPT protein structures and the putative (and biologically active) GPT homolog from Danio rerio (Zebra fish) (see Example 22), other non-plant GPT homologs may be used in preparing GPT transgenes for use in generating the transgenic plants of the invention.

When individually compared (by BLAST alignment) to the Arabidopsis mature protein sequence provided in SEQ ID NO: 30, the following sequence identities and homologies (BLAST “positives”, including similar amino acids) were obtained for the following mature GPT protein sequences:

| [SEQ ID] or FIG. NO. | ORIGIN | % IDENTITY | % POSITIVE |
|---|---|---|---|
| [31] | Grape | 84 | 93 |
| [32] | Rice | 83 | 91 |
| [33] | Soybean | 83 | 93 |
| [34] | Barley | 82 | 91 |
| [35] | Zebra fish | 83 | 92 |
| [36] | Bamboo | 81 | 90 |
| FIG. 2 | Corn | 79 | 90 |
| FIG. 2 | Castor | 84 | 93 |
| FIG. 2 | Poplar | 85 | 93 |

Underscoring the conserved nature of the structure of the GPT protein across most plant species, the conservation seen within the above plant species extends to the non-human putative GPTs from Zebra fish and Chlamydomonas. In the case of Zebra fish, the extent of identity is very high (83% amino acid sequence identity with the mature Arabidopsis GPT of SEQ ID NO: 30, and 92% homologous taking similar amino acid residues into account). The Zebra fish mature GPT was confirmed by expressing it in E. coli and demonstrating biological activity (synthesis of 2-oxoglutaramate).

In order to determine whether putative GPT homologs would be suitable for generating the growth-enhanced transgenic plants of the invention, one need initially express the coding sequence thereof in E. coli or another suitable host and determine whether the 2-oxoglutaramate signal metabolite is synthesized at increased levels (see Examples 19-23). Where such an increase is demonstrated, the coding sequence may then be introduced into both homologous plant hosts and heterologous plant hosts, and growth characteristics evaluated. Any assay that is capable of detecting 2-oxoglutaramate with specificity may be used for this purpose, including without limitation the NMR and HPLC assays described in Example 2, infra. In addition, assays which measure GPT activity directly may be employed, such as the GPT activity assay described in Example 7.

Any plant GPT with 2-oxoglutaramate synthesis activity may be used to transform plant cells in order to generate transgenic plants of the invention. There appears to be a high level of structural homology among plant species, which appears to extend beyond plants, as evidenced by the close homology between various plant GPT proteins and the putative Zebra fish GPT homolog. Therefore, various plant GPT genes may be used to generate growth-enhanced transgenic plants in a variety of heterologous plant species. In addition, GPT transgenes expressed in a homologous plant would be expected to result in the desired enhanced-growth characteristics as well (i.e., rice glutamine transaminase over-expressed in transgenic rice plants), although it is possible that regulation within a homologous cell may attenuate the expression of the transgene in some fashion that may not be operable in a heterologous cell.

### Glutamine Synthetase (GS) Transgene:

In the practice of the invention, the glutamine synthetase (GS) gene functions as one of at least two transgenes incorporated into the transgenic plants of the invention (GPT being the other of the two).

Glutamine synthetase plays a key role in nitrogen metabolism in plants, as well as in animals and bacteria. The GS enzyme catalyzes the addition of ammonium to glutamate to synthesize glutamine in an ATP-dependent reaction. GS enzymes from assorted species show highly conserved amino acid residues considered to be important for active site function, indicating that GS enzymes function similarly (for review, see Eisenberg et al., Biochimica et Biophysica Acta, 1477:122 145, 2000).

GS is distributed in different subcellular locations (chloroplast and cytoplasm) and is found in various plant tissues, including leaf, root, shoot, seeds and fruits. There are two major isoforms of plant GS: the cystolic isoform (GS1) and the plastidic (chloroplastic) isoform (GS2). GS2 is principally found in leaf tissue and functions in the assimilation of ammonia produced by photorespiration or by nitrate reduction. GS1 is mainly found in leaf and root tissue, typically exists in a number of different isoforms in higher plants, and functions to assimilate ammonia produced by all other physiological processes (Coruzzi, 1991, Plant Science 74: 145-155; McGrath and Coruzzi, 1991, Plant J. 1(3): 275-280; Lam et al., 1996, Ann. Rev. Plant Physiol. Plant Mol. Biol. 47: 569-593; Stitt, 1999, Curr. Op. Plant Biol. 2: 178-186; Oliveira et al., 2001, Brazilian J. Med. Biol. Res. 34: 567-575). Multiple GS genes are associated with a complex promoter repertoire which enable the expression of GS in an organ and tissue specific manner, as well as in an environmental factor-dependent manner.

Plant glutamine synthetase consists of eight subunits, and the native enzyme in plants has a molecular mass ranging from 320 to 380 kD, each subunit having a molecular mass of between 38 and 45 kD. The GS1 genes of several plants, especially legumes, have been cloned and sequenced (Tischer et al., 1986, Mol Gen Genet. 203: 221-229; Gebhardt et al., 1986, EMBO J. 5: 1429-1435; Tingey et al., 1987, EMBO J. 6: 1-9; Tingey et al., 1988, J Biol Chem. 263: 9651-9657; Bennett et al., 1989, Plant Mol Biol. 12: 553-565; Boron and Legocki, 1993, Gene 136: 95-102; Roche et al., 1993, Plant Mol Biol. 22: 971-983; Marsolier et al., 1995, Plant Mol Biol. 27: 1-15; Temple et al., 1995, Mol Plant-Microbe Interact. 8: 218-227). All have been found to be encoded by nuclear genes (for review, see, Morey et al., 2002, Plant Physiol. 128(1): 182-193).

Chloroplastic GS2 appears to be encoded by a single gene, while various cystoloic GS1 isoforms are encoded within multigene families (Tingey et al., 1987, supra; Sakamoto et al., 1989, Plant Mol. Biol. 13: 611-614; Brears et al, 1991, supra; Li et al., 1993, Plant Mol. Biol., 23:401-407; Dubois et al., 1996, Plant Mol. Biol., 31:803-817; Lam et al., 1996, supra). GS1 multigene families appear to encode different subunits which may combine to form homo- or hetero-octamers, and the different members show a unique expression pattern suggesting that the gene members are differentially regulated, which may relate to the various functional roles of glutamine synthetase plays in overall nitrogen metabolism (Gebhardt et al., 1986, supra; Tingey et al., 1987, supra; Bennett et al., 1989, supra; Walker and Coruzzi, 1989, supra; Peterman and Goodman, 1991, Mol Gen Genet. 1991; 330:145-154.; Marsolier et al., 1995, supra; Temple et al., 1995, supra; Dubois et al., 1996, supra).

In one embodiment, a GS1 gene coding sequence is employed to generate GS transgene constructs. In particular embodiments, further described in the Examples, infra, the Alfalfa or Arabidopsis GS1 gene coding sequence is used to generate a transgene construct that may be used to generate a transgenic plant expressing the GS1 transgene. As an example, such a construct may be used to transform Agrobacteria. The transformed Agrobacteria are then used to generate T₀ transgenic plants. Example 5 demonstrates the generation of T₀ GS1 transgenic tobacco plants using this approach. Similarly, Examples 6 and 17 demonstrates the generation of T₀ GS1 transgenic tomato plants, Example 8 demonstrates the generation of T₀ GS1 transgenic pepper plants, Examples 9 and 10 demonstrate the generation of T₀ GS1 transgenic bean plants, Examples 11 and 12 demonstrate the generation of T₀ GS1 transgenic cowpea plants, Example 13 demonstrates the generation of T₀ GS1 transgenic alfalfa plants, Example 14 demonstrates the generation of T₀ GS1 transgenic cantaloupe plants, Example 15 demonstrates the generation of T₀ GS1 transgenic pumpkin plants, Example 16 demonstrates the generation of T₀ GS1 transgenic Arabidopsis plants, and Example 18 demonstrates the generation of T₀ GS1 transgenic Cantaloupe plants.

### Transcription Terminators:

In preferred embodiments, a 3′ transcription termination sequence is incorporated downstream of the transgene in order to direct the termination of transcription and permit correct polyadenylation of the mRNA transcript. Suitable transcription terminators are those which are known to function in plants, including without limitation, the nopaline synthase (NOS) and octopine synthase (OCS) genes of Agrobacterium tumefaciens, the T7 transcript from the octopine synthase gene, the 3′ end of the protease inhibitor I or II genes from potato or tomato, the CaMV 35S terminator, the tml terminator and the pea rbcS E9 terminator. In addition, a gene's native transcription terminator may be used. In specific embodiments, described by way of the Examples, infra, the nopaline synthase transcription terminator is employed.

### Selectable Markers:

Selectable markers are typically included in transgene expression vectors in order to provide a means for selecting transformants. While various types of markers are available, various negative selection markers are typically utilized, including those which confer resistance to a selection agent that inhibits or kills untransformed cells, such as genes which impart resistance to an antibiotic (such as kanamycin, gentamycin, anamycin, hygromycin and hygromycinB) or resistance to a herbicide (such as sulfonylurea, gulfosinate, phosphinothricin and glyphosate): Screenable markers include, for example, genes encoding β-glucuronidase (Jefferson, 1987, Plant Mol. Biol. Rep 5: 387-405), genes encoding luciferase (Ow et al., 1986, Science 234: 856-859) and various genes encoding proteins involved in the production or control of anthocyanin pigments (See, for example, U.S. Pat. No. 6,573,432). The E. coli glucuronidase gene (gus, gusA or uidA) has become a widely used selection marker in plant transgenics, largely because of the glucuronidase enzyme's stability, high sensitivity and ease of detection (e.g., fluorometric, spectrophotometric, various histochemical methods). Moreover, there is essentially no detectable glucuronidase inmost higher plant species.

### Transformation Methodologies and Systems:

Various methods for introducing the transgene expression vector constructs of the invention into a plant or plant cell are well known to those skilled in the art, and any capable of transforming the target plant or plant cell may be utilized.

Agrobacterium-mediated transformation is perhaps the most common method utilized in plant transgenics, and protocols for Agrobacterium-mediated transformation of a large number of plants are extensively described in the literature (see, for example, Agrobacterium Protocols, Wan, ed., Humana Press, 2^{nd} edition, 2006). Agrobacterium tumefaciens is a Gram negative soil bacteria that causes tumors (Crown Gall disease) in a great many dicot species, via the insertion of a small segment of tumor-inducing DNA (“T-DNA”, ‘transfer DNA’) into the plant cell, which is incorporated at a semi-random location into the plant genome, and which eventually may become stably incorporated there. Directly repeated DNA sequences, called T-DNA borders, define the left and the right ends of the T-DNA. The T-DNA can be physically separated from the remainder of the Ti-plasmid, creating a ‘binary vector’ system.

Agrobacterium transformation may be used for stably transforming dicots, monocots, and cells thereof (Rogers et al., 1986, Methods Enzymol., 118: 627-641; Hernalsteen et al., 1984, EMBO J., 3: 3039-3041; Hoykass-Van Slogteren et al., 1984, Nature, 311: 763-764; Grimsley et al., 1987, Nature 325: 167-1679; Boulton et al., 1989, Plant Mol. Biol. 12: 31-40; Gould et al., 1991, Plant Physiol. 95: 426-434). Various methods for introducing DNA into Agrobacteria are known, including electroporation, freeze/thaw methods, and triparental mating. The most efficient method of placing foreign DNA into Agrobacterium is via electroporation (Wise et al., 2006, Three Methods for the Introduction of Foreign DNA into Agrobacterium, Methods in Molecular Biology, vol. 343: Agrobacterium Protocols, 2/e, volume 1; Ed., Wang, Humana Press Inc., Totowa, N.J., pp. 43-53). In addition, given that a large percentage of T-DNAs do not integrate, Agrobacterium-mediated transformation may be used to obtain transient expression of a transgene via the transcriptional competency of unincorporated transgene construct molecules (Helens et al., 2005, Plant Methods 1:13).

A large number of Agrobacterium transformation vectors and methods have been described (Karimi et al., 2002, Trends Plant Sci. 7(5): 193-5), and many such vectors may be obtained commercially (for example, Invitrogen, Carlsbad, Calif.). In addition, a growing number of “open-source” Agrobacterium transformation vectors are available (for example, pCambia vectors; Cambia, Canberra, Australia). See, also, subsection herein on TRANSGENE CONSTRUCTS, supra. In a specific embodiment described further in the Examples, a pMON316-based vector was used in the leaf disc transformation system of Horsch et. al. (Horsch et al., 1995, Science 227:1229-1231) to generate growth enhanced transgenic tobacco and tomato plants.

Other commonly used transformation methods that may be employed in generating the transgenic plants of the invention include, without limitation microprojectile bombardment, or biolistic transformation methods, protoplast transformation of naked DNA by calcium, polyethylene glycol (PEG) or electroporation (Paszkowski et al., 1984, EMBO J. 3: 2727-2722; Potrykus et al., 1985, Mol. Gen. Genet. 199: 169-177; Fromm et al., 1985, Proc. Nat. Acad. Sci. USA 82: 5824-5828; Shimamoto et al., 1989, Nature, 338: 274-276.

Biolistic transformation involves injecting millions of DNA-coated metal particles into target cells or tissues using a biolistic device (or “gene gun”), several kinds of which are available commercially. Once inside the cell, the DNA elutes off the particles and a portion may be stably incorporated into one or more of the cell's chromosomes (for review, see Kikkert et al., 2005, Stable Transformation of Plant Cells by Particle Bombardment/Biolistics, in: Methods in Molecular Biology, vol. 286: Transgenic Plants: Methods and Protocols, Ed. L. Pena, Humana Press Inc., Totowa, N.J.).

Electroporation is a technique that utilizes short, high-intensity electric fields to permeabilize reversibly the lipid bilayers of cell membranes (see, for example, Fisk and Dandekar, 2005, Introduction and Expression of Transgenes in Plant Protoplasts, in: Methods in Molecular Biology, vol. 286: Transgenic Plants: Methods and Protocols, Ed. L. Peña, Humana Press Inc., Totowa, N.J., pp. 79-90; Fromm et al., 1987, Electroporation of DNA and RNA into plant protoplasts, in Methods in Enzymology, Vol. 153, Wu and Grossman, eds., Academic Press, London, UK, pp. 351-366; Joersbo and Brunstedt, 1991, Electroporation: mechanism and transient expression, stable transformation and biological effects in plant protoplasts. Physiol. Plant. 81, 256-264; Bates, 1994, Genetic transformation of plants by protoplast electroporation. Mol. Biotech. 2: 135-145; Dillen et al., 1998, Electroporation-mediated DNA transfer to plant protoplasts and intact plant tissues for transient gene expression assays, in Cell Biology, Vol. 4, ed., Celis, Academic Press, London, UK, pp. 92-99). The technique operates by creating aqueous pores in the cell membrane, which are of sufficiently large size to allow DNA molecules (and other macromolecules) to enter the cell, where the transgene expression construct (as T-DNA) may be stably incorporated into plant genomic DNA, leading to the generation of transformed cells that can subsequently be regenerated into transgenic plants.

Newer transformation methods include so-called “floral dip” methods, which offer the promise of simplicity, without requiring plant tissue culture, as is the case with all other commonly used transformation methodologies (Bent et al., 2006, Arabidopsis thaliana Floral Dip Transformation Method, Methods Mol Biol, vol. 343: Agrobacterium Protocols, 2/e, volume 1; Ed., Wang, Humana Press Inc., Totowa, N.J., pp. 87-103; Clough and Bent, 1998, Floral dip: a simplified method for Agrobacterium-mediated transformation of Arabidopsis thaliana, Plant J. 16: 735-743). However, with the exception of Arabidopsis, these methods have not been widely used across a broad spectrum of different plant species. Briefly, floral dip transformation is accomplished by dipping or spraying flowering plants in with an appropriate strain of Agrobacterium tumefaciens. Seeds collected from these T₀ plants are then germinated under selection to identify transgenic T₁ individuals. Example 16 demonstrated floral dip inoculation of Arabidopsis to generate transgenic Arabidopsis plants.

Other transformation methods include those in which the developing seeds or seedlings of plants are transformed using vectors such as Agrobacterium vectors. For example, as exemplified in Example 8, such vectors may be used to transform developing seeds by injecting a suspension or mixture of the vector (i.e., Agrobacteria) directly into the seed cavity of developing pods (i.e., pepper pods, bean pods, pea pods and the like). Seedlings may be transformed as described for Alfalfa in Example 13. Germinating seeds may be transformed as described for Camelina in Example 18. Intra-fruit methods, in which the vector is injected into fruit or developing fruit, may be used as described for Cantaloupe melons in Example 14 and pumpkins in Example 15.

Still other transformation methods include those in which the flower structure is targeted for vector inoculation, such as the flower inoculation methods described for beans in Examples 9 and 10, peas in Examples 11 and 12 and tomatoes in Example 17.

The foregoing plant transformation methodologies may be used to introduce transgenes into a number of different plant cells and tissues, including without limitation, whole plants, tissue and organ explants including chloroplasts, flowering tissues and cells, protoplasts, meristem cells, callus, immature embryos and gametic cells such as microspores, pollen, sperm and egg cells, tissue cultured cells of any of the foregoing, any other cells from which a fertile regenerated transgenic plant may be generated. Callus is initiated from tissue sources including, but not limited to, immature embryos, seedling apical meristems, microspores and the like. Cells capable of proliferating as callus are also recipient cells for genetic transformation.

Methods of regenerating individual plants from transformed plant cells, tissues or organs are known and are described for numerous plant species.

As an illustration, transformed plantlets (derived from transformed cells or tissues) are cultured in a root-permissive growth medium supplemented with the selective agent used in the transformation strategy (i.e., and antibiotic such as kanamycin). Once rooted, transformed plantlets are then transferred to soil and allowed to grow to maturity. Upon flowering, the mature plants are preferably selfed (self-fertilized), and the resultant seeds harvested and used to grow subsequent generations. Examples 3-6 describe the regeneration of transgenic tobacco and tomato plants.

T₀ transgenic plants may be used to generate subsequent generations (e.g., T₁, T₂, etc.) by selfing of primary or secondary transformants, or by sexual crossing of primary or secondary transformants with other plants (transformed or untransformed). For example, as described in Example 7, infra, individual plants over expressing the Alfalfa GS1 gene and outperforming wildtype plants were crossed with individual plants over-expressing the Arabidopsis GPT gene and outperforming wildtype plants, by simple sexual crossing using manual pollen transfer. Reciprocal crosses were made such that each plant served as the male in a set of crosses and each plant served as the female in a second set of crosses. During the mature plant growth stage, the plants are typically examined for growth phenotype, CO₂ fixation rate, etc. (see following subsection).

### Selection of Growth-Enhanced Transgenic Plants:

Transgenic plants may be selected, screened and characterized using standard methodologies. The preferred transgenic plants of the invention will exhibit one or more phenotypic characteristics indicative of enhanced growth and/or other desirable agronomic properties. Transgenic plants are typically regenerated under selective pressure in order to select transformants prior to creating subsequent transgenic plant generations. In addition, the selective pressure used may be employed beyond T₀ generations in order to ensure the presence of the desired transgene expression construct or cassette.

T₀ transformed plant cells, calli, tissues or plants may be identified and isolated by selecting or screening for the genetic composition of and/or the phenotypic characteristics encoded by marker genes contained in the transgene expression construct used for the transformation. For example, selection may be conducted by growing potentially-transformed plants, tissues or cells in a growth medium containing a repressive amount of antibiotic or herbicide to which the transforming genetic construct can impart resistance. Further, the transformed plant cells, tissues and plants can be identified by screening for the activity of marker genes (i.e., β-glucuronidase) which may be present in the transgene expression construct.

Various physical and biochemical methods may be employed for identifying plants containing the desired transgene expression construct, as is well known. Examples of such methods include Southern blot analysis or various nucleic acid amplification methods (i.e., PCR) for identifying the transgene, transgene expression construct or elements thereof, Northern blotting, S1 RNase protection, reverse transcriptase PCR (RT-PCR) amplification for detecting and determining the RNA transcription products, and protein gel electrophoresis, Western blotting, immunoprecipitation, enzyme immunoassay, and the like may be used for identifying the protein encoded and expressed by the transgene.

In another approach, expression levels of genes, proteins and/or metabolic compounds that are know to be modulated by transgene expression in the target plant may be used to identify transformants. In one embodiment of the present invention, increased levels of the signal metabolite 2-oxoglutaramate may be used to screen for desirable transformants, as exemplified in the Examples. Similarly, increased levels of GPT and/or GS activity may be assayed, as exemplified in the Examples.

Ultimately, the transformed plants of the invention may be screened for enhanced growth and/or other desirable agronomic characteristics. Indeed, some degree of phenotypic screening is generally desirable in order to identify transformed lines with the fastest growth rates, the highest seed yields, etc., particularly when identifying plants for subsequent selfing, cross-breeding and back-crossing. Various parameters may be used for this purpose, including without limitation, growth rates, total fresh weights, dry weights, seed and fruit yields (number, weight), seed and/or seed pod sizes, seed pod yields (e.g., number, weight), leaf sizes, plant sizes, increased flowering, time to flowering, overall protein content (in seeds, fruits, plant tissues), specific protein content (i.e., GS), nitrogen content, free amino acid, and specific metabolic compound levels (i.e., 2-oxoglutaramate). Generally, these phenotypic measurements are compared with those obtained from a parental identical or analogous plant line, an untransformed identical or analogous plant, or an identical or analogous wild-type plant (i.e., a normal or parental plant). Preferably, and at least initially, the measurement of the chosen phenotypic characteristic(s) in the target transgenic plant is done in parallel with measurement of the same characteristic(s) in a normal or parental plant. Typically, multiple plants are used to establish the phenotypic desirability and/or superiority of the transgenic plant in respect of any particular phenotypic characteristic.

Preferably, initial transformants are selected and then used to generate T₁ and subsequent generations by selfing (self-fertilization), until the transgene genotype breeds true (i.e., the plant is homozygous for the transgene). In practice, this is accomplished by screening at each generation for the desired traits and selfing those individuals, often repeatedly (i.e., 3 or 4 generations). As exemplified herein, transgenic plant lines propagated through at least one sexual generation (Tobacco, Arabidopsis, Tomato) demonstrated higher transgene product activities compared to lines that did not have the benefit of sexual reproduction and the concomitant increase in transgene copy number.

Stable transgenic lines may be crossed and back-crossed to create varieties with any number of desired traits, including those with stacked transgenes, multiple copies of a transgene, etc. Various common breeding methods are well know to those skilled in the art (see, e.g., Breeding Methods for Cultivar Development, Wilcox J. ed., American Society of Agronomy, Madison Wis. (1987)). Additionally, stable transgenic plants may be further modified genetically, by transforming such plants with further transgenes or additional copies of the parental transgene. Also contemplated are transgenic plants created by single transformation events which introduce multiple copies of a given transgene or multiple transgenes.

In a another aspect, the invention provides transgenic plants characterized by increased nitrogen use efficiency. Nitrogen use efficiency may be expressed as plant yield per given amount of nitrogen. In the Examples provided herein, the transgene and control plants all received the same nutrient solutions in the same amounts. The transgenic plants were consistently characterized by higher yields, and thus have higher nitrogen use efficiencies.

In yet another aspect, the invention provides transgenic plants and seeds thereof with increased tolerance to high salt growth conditions. This aspect of the invention is exemplified by Example 24, which describes the germination of transgenic tobacco plant seeds in very high salt conditions (200 mM NaCl). While counterpart wild type tobacco seeds germinated at a rate of only about 10%, on average, the transgenic tobacco seeds achieved nearly the same rate of germination obtained under no salt conditions for both transgenic and wild type seeds, or about 92%.

### EXAMPLES

Various aspects of the invention are further described and illustrated by way of the several examples which follow, none of which are intended to limit the scope of the invention.

### Example 1

### Isolation of Arabidopsis Gluamine Phenylpyruvate Transaminase (GPT) Gene

In an attempt to locate a plant enzyme that is directly involved in the synthesis of the signal metabolite 2-oxoglutaramate, applicants hypothesized that the putative plant enzyme might bear some degree of structural relationship to a human protein that had been characterized as being involved in the synthesis of 2-oxoglutaramate. The human protein, glutamine transaminase K (E.C. 2.6.1.64) (also referred in the literature as cysteine conjugate β-lyase, kyneurenine aminotransferase, glutamine phenylpyruvate transaminase, and other names), had been shown to be involved in processing of cysteine conjugates of halogenated xenobiotics (Perry et al., 1995, FEBS Letters 360:277-280). Rather than having an activity involved in nitrogen assimilation, however, human cysteine conjugate β-lyase has a detoxifying activity in humans, and in animals (Cooper and Meister, 1977, supra). Nevertheless, the potential involvement of this protein in the synthesis of 2-oxoglutaramate was of interest.

Using the protein sequence of human cysteine conjugate β-lyase, a search against the TIGR Arabidopsis plant database of protein sequences identified one potentially related sequence, a polypeptide encoded by a partial sequence at the Arabidopsis gene locus at At1q77670, sharing approximately 36% sequence homology/identity across aligned regions.

The full coding region of the gene was then amplified from an Arabidopsis cDNA library (Stratagene) with the following primer pair:

[SEQ ID NO: 37]

5′-CCCATCGATGTACC TGGACATAAATGGTGTGATG-3′

[SEQ ID NO: 38]

5′-GATGGTACCTCAGACTTTTCTCTTAAGCTTCTGCTTC-3′

These primers were designed to incorporate Cla I (ATCGAT) and Kpn I (GGTACC) restriction sites to facilitate subsequent subcloning into expression vectors for generating transgenic plants. Takara ExTaq DNA polymerase enzyme was used for high fidelity PCR using the following conditions: initial denaturing 94° C. for 4 minutes, 30 cycles of 94° C. 30 second, annealing at 55° C. for 30 seconds, extension at 72° C. for 90 seconds, with a final extension of 72° C. for 7 minutes. The amplification product was digested with Cla I and Kpn I restriction enzymes, isolated from an agarose gel electrophoresis and ligated into vector pMon316 (Rogers, et. al. 1987 Methods in Enzymology 153:253-277) which contains the cauliflower mosaic virus (CaMV, also CMV) 35S constitutive promoter and the nopaline synthase (NOS) 3′ terminator. The ligation product was transformed into DH5α cells and transformants sequenced to verify the insert.

A 1.3 kb cDNA was isolated and sequenced, and found to encode a full length protein of 440 amino acids in length, including a putative chloroplast signal sequence.

### Example 2

### Production of Biologically Active Recombinant Arabidopsis Glutamine Phenylpyruvate Transaminase (GPT)

To test whether the protein encoded by the cDNA isolated as described in Example 1, supra, is capable of catalyzing the synthesis of 2-oxoglutaramate, the cDNA was expressed in E. coli, purified, and assayed for its ability to synthesize 2-oxoglutaramate using a standard method.

NMR Assay for 2-oxoglutaramate:

Briefly, the resulting purified protein was added to a reaction mixture containing 150 mM Tris-HCl, pH 8.5, 1 mM beta mercaptoethanol, 200 mM glutamine, 100 mM glyoxylate and 200 μM pyridoxal 5′-phosphate. The reaction mixture without added test protein was used as a control. Test and control reaction mixtures were incubated at 37° C. for 20 hours, and then clarified by centrifugation to remove precipitated material. Supernatants were tested for the presence and amount of 2-oxoglutaramate using ¹³C NMR with authentic chemically synthesized 2-oxoglutaramate as a reference. The products of the reaction are 2-oxoglutaramate and glycine, while the substrates (glutamine and glyoxylate) diminish in abundance. The cyclic 2-oxoglutaramate gives rise to a distinctive signal allowing it to be readily distinguished from the open chain glutamine precursor.

HPLC Assay for 2-oxoglutaramate:

An alternative assay for GPT activity uses HPLC to determine 2-oxoglutaramate production, following a modification of Calderon et al., 1985, J Bacteriol 161(2): 807-809. Briefly, a modified extraction buffer consisting of 25 mM Tris-HCl pH 8.5, 1 mM EDTA, 20 μM FAD, 10 mM Cysteine, and ˜1.5% (v/v) Mercaptoethanol. Tissue samples from the test material (i.e., plant tissue) are added to the extraction buffer at approximately a 1/3 ratio (w/v), incubated for 30 minutes at 37° C., and stopped with 200 μl of 20% TCA. After about 5 minutes, the assay mixture is centrifuged and the supernatant used to quantify 2-oxoglutaramate by HPLC, using an ION-300 7.8 mm ID×30 cm L column, with a mobile phase in 0.01N H₂SO₄, a flow rate of approximately 0.2 ml/min, at 40° C. Injection volume is approximately 20 μl, and retention time between about 38 and 39 minutes. Detection is achieved with 210 nm UV light.

### Results Using NMR Assay:

This experiment revealed that the test protein was able to catalyze the synthesis of 2-oxoglutaramate. Therefore, these data indicate that the isolated cDNA encodes a glutamine phenylpyruvate transaminase that is directly involved in the synthesis of 2-oxoglutaramate in plants. Accordingly, the test protein was designated Arabidopsis glutamine phenylpyruvate transaminase, or “GPT”.

The nucleotide sequence of the Arabidopsis GPT coding sequence is shown in the Table of Sequences, SEQ ID NO. 1. The translated amino acid sequence of the GPT protein is shown in SEQ ID NO. 2.

### Example 3

### Creation of Transgenic Tobacco Plants Over-Expressing Arabidopsis GPT

Generation of Plant Expression Vector pMON-PJU:

Briefly, the plant expression vector pMon316-PJU was constructed as follows. The isolated cDNA encoding Arabidopsis GPT (Example 1) was cloned into the ClaI-KpnI polylinker site of the pMON316 vector, which places the GPT gene under the control of the constitutive cauliflower mosaic virus (CaMV) 35S promoter and the nopaline synthase (NOS) transcriptional terminator. A kanamycin resistance gene was included to provide a selectable marker.

### Agrobacterium-Mediated Plant Transformations:

pMON-PJU and a control vector pMon316 (without inserted DNA) were transferred to Agrobacterium tumefaciens strain pTiTT37ASE using a standard electroporation method (McCormac et al., 1998, Molecular Biotechnology 9:155-159), followed by plating on LB plates containing the antibiotics spectinomycin (100 micro gm/ml) and kanamycin (50 micro gm/ml). Antibiotic resistant colonies of Agrobacterium were examined by PCR to assure that they contained plasmid.

Nicotiana tabacum cv. Xanthi plants were transformed with pMON-PJU transformed Agrobacteria using the leaf disc transformation system of Horsch et. al. (Horsch et al., 1995, Science 227:1229-1231). Briefly, sterile leaf disks were inoculated and cultured for 2 days, then transferred to selective MS media containing 100 μg/ml kanamycin and 500 μg/ml clafaran. Transformants were confirmed by their ability to form roots in the selective media.

### Generation of GPT Transgenic Tobacco Plants:

Sterile leaf segments were allowed to develop callus on Murashige & Skoog (M&S) media from which the transformant plantlets emerged. These plantlets were then transferred to the rooting-permissive selection medium (M&S medium with kanamycin as the selection agent). The healthy, and now rooted, transformed tobacco plantlets were then transferred to soil and allowed to grow to maturity and upon flowering the plants were selfed and the resultant seeds were harvested. During the growth stage the plants had been examined for growth phenotype and the CO₂ fixation rate was measured for many of the young transgenic plants.

### Production of T1 and T2 Generation GPT Transgenic Plants:

Seeds harvested form the T₀ generation of the transgenic tobacco plants were germinated on M&S media containing kanamycin (100 mg/L) to enrich for the transgene. At least one fourth of the seeds did not germinate on this media (kanamycin is expected to inhibit germination of the seeds without resistance that would have been produced as a result of normal genetic segregation of the gene) and more than half of the remaining seeds were removed because of demonstrated sensitivity (even mild) to the kanamycin.

The surviving plants (T₁ generation) were thriving and these plants were then selfed to produce seeds for the T₂ generation. Seeds from the T₁ generation were germinated on MS media supplemented for the transformant lines with kanamycin (10 mg/liter). After 14 days they were transferred to sand and provided quarter strength Hoagland's nutrient solution supplemented with 25 mM potassium nitrate. They were allowed to grow at 24° C. with a photoperiod of 16 h light and 8 hr dark with a light intensity of 900 micromoles per meter squared per second. They were harvested 14 days after being transferred to the sand culture.

### Characterization of GPT Transgenic Plants:

Harvested transgenic plants (both GPT transgenes and vector control transgenes) were analyzed for glutamine sythetase activity in root and leaf, whole plant fresh weight, total protein in root and leaf, and CO₂ fixation rate (Knight et al., 1988, Plant Physiol. 88: 333). Non-transformed, wild-type A. tumefaciens plants were also analyzed across the same parameters in order to establish a baseline control.

Growth characteristic results are tabulated below in Table I. Additionally, a photograph of the GPT transgenic plant compared to a wild type control plant is shown in FIG. 2 (together with GS1 transgenic tobacco plant, see Example 5). Across all parameters evaluated, the GPT transgenic tobacco plants showed enhanced growth characteristics. In particular, the GPT transgenic plants exhibited a greater than 50% increase in the rate of CO₂ fixation, and a greater than two-fold increase in glutamine synthetase activity in leaf tissue, relative to wild type control plants. In addition, the leaf-to-root GS ratio increased by almost three-fold in the transaminase transgenic plants relative to wild type control. Fresh weight and total protein quantity also increased in the transgenic plants, by about 50% and 80% (leaf), respectively, relative to the wild type control. These data demonstrate that tobacco plants overexpressing the Arabidopsis GPT transgene achieve significantly enhanced growth and CO₂ fixation rates.

TABLE I

| | |
|---|---|
| Leaf | Root |

Protein mg/gram fresh weight

| | | |
|---|---|---|
| Wild type - control | 8.3 | 2.3 |
| Line PN1-8 a second control | 8.9 | 2.98 |
| Line PN9-9 | 13.7 | 3.2 |

Glutamine Synthetase activity, micromoles/min/mg protein

| | | |
|---|---|---|
| Wild type (Ratio of leaf:root = 4.1:1) | 4.3 | 1.1 |
| PN1-8 (Ratio of leaf:root = 4.2:1) | 5.2 | 1.3 |
| PN9-9 (Ratio of leaf:root = 10.9:1) | 10.5 | 0.97 |

Whole Plant Fresh Weight, g

| | |
|---|---|
| Wild type | 21.7 |
| PN1-8 | 26.1 |
| PN9-9 | 33.1 |

CO₂ Fixation Rate, umole/m2/sec

| | |
|---|---|
| Wild type | 8.4 |
| PN1-8 | 8.9 |
| PN9-9 | 12.9 |

Data = average of three plants
Wild type - Control plants; not regenerated or transformed.
PN1 lines were produced by regeneration after transformation using a construct without inserted gene. A control against the processes of regeneration and transformation.
PN9 lines were produced by regeneration after transformation using a construct with the Arabidopsis GPT gene.

### Example 4

### Generation of Transgenic Tomato Plants Carrying Arabidopsis GPT Transgene

Transgenic Lycopersicon esculentum (Micro-Tom Tomato) plants carrying the Arabidopsis GPT transgene were generated using the vectors and methods described in Example 3. T₀ transgenic tomato plants were generated and grown to maturity. Initial growth characteristic data of the GPT transgenic tomato plants is presented in Table II. The transgenic plants showed significant enhancement of growth rate, flowering, and seed yield in relation to wild type control plants. In addition, the transgenic plants developed multiple main stems, whereas wild type plants developed with a single main stem. A photograph of a GPT transgenic tomato plant compared to a wild type plant is presented in FIG. 3 (together with GS1 transgenic tomato plants, see Example 6).

TABLE II

| Growth | Wildtype | GPT Transgenic |
|---|---|---|
| Characteristics | Tomato | Tomato |
| Stem height, cm | 6.5 | 18, 12, 11 major stems |
| Stems | 1 | 3 major, 0 other |
| Buds | 2 | 16 |
| Flowers | 8 | 12 |
| Fruit | 0 | 3 |

### Example 5

### Generation of Transgenic Tobacco Plants Overexpressing Alfalfa GS1

Generation of Plant Expression Vector pGS111:

Transgenic tobacco plants overexpressing the Alfalfa GS1 gene were generated as previously described (Temple et al., 1993, Mol. Gen. Genetics 236: 315-325). Briefly, the plant expression vector pGS111 was constructed by inserting the entire coding sequence together with extensive regions of both the 5′ and 3′ untranslated regions of the Alfalfa GS1 gene [SEQ ID NO: 3] (DasSarma at al., 1986, Science, Vol 232, Issue 4755, 1242-1244) into pMON316 (Rogers et al., 1987, supra), placing the transgene under the control of the constitutive cauliflower mosaic virus (CaMV) 35S promoter and the nopaline synthase (NOS) transcriptional terminator. A kanamycin resistance gene was included to provide a selectable marker.

### Generation of GS1 Transformants:

pGS111 was transferred to Agrobacterium tumefaciens strain pTiTT37ASE using triparental mating as described (Rogers et al., 1987, supra; Unkefer et al., U.S. Pat. No. 6,555,500). Nicotiana tabacum cv. Xanthi plants were transformed with pGS111 transformed Agrobacteria using the leaf disc transformation system of Horsch et. al. (Horsch et al., 1995, Science 227:1229-1231). Transformants were selected and regenerated on MS medium containing 100 μg/ml kanamycin. Shoots were rooted on the same medium (with kanamycin, absent hormones) and transferred to potting soil:perlite:vermiculite (3:1:1), grown to maturity, and allowed to self. Seeds were harvested from this T₀ generation, and subsequent generations produced by selfing and continuing selection with kanamycin. The best growth performers were used to yield a T3 progeny for crossing with the best performing GPT over-expressing lines identified as described in Example 3. A photograph of the GS1 transgenic plant compared to a wild type control plant is shown in FIG. 2 (together with GPT transgenic tobacco plant, see Example 3)

### Example 6

### Generation of Transgenic Tomato Plants Carrying Alfalfa GS1 Transgene

Transgenic Lycopersicon esculentum (Micro-Tom Tomato) plants carrying the Alfalfa GS1 transgene were generated using the vector described in Example 5 and a transformation protocol essentially as described (Sun et al., 2006. Plant Cell Physiol. 46(3) 426-31). T₀ transgenic tomato plants were generated and grown to maturity. Initial growth characteristic data of the GPT transgenic tomato plants is presented in Table III. The transgenic plants showed significant enhancement of growth rate, flowering, and seed yield in relation to wild type control plants. In addition, the transgenic plants developed multiple main stems, whereas wild type plants developed with a single main stem. A photograph of a GS1 transgenic tomato plant compared to a wild type plant is presented in FIG. 3 (together with GPT transgenic tomato plant, see Example 4).

TABLE III

| Growth | Wildtype | GS1 Transgenic |
|---|---|---|
| Characteristics | Tomato | Tomato |
| Stem height, cm | 6.5 | 16, 7, 5 major stems |
| Stems | 1 | 3 major, 3 med, 1 sm |
| Buds | 2 | 2 |
| Flowers | 8 | 13 |
| Fruit | 0 | 4 |

### Example 7

### Generation of Double Transgenic Tobacco Plants Carrying GS1 and GPT Transgenes

In an effort to determine whether the combination of GS1 and GPT transgenes in a single transgenic plant might improve the extent to which growth and other agronomic characteristics may be enhanced, a number of sexual crosses between high producing lines of the single transgene (GS1 or GPT) transgenic plants were carried out. The results obtained are dramatic, as these crosses repeatedly generated progeny plants having surprising and heretofore unknown increases in growth rates, biomass yield, and seed production.

### Materials and Methods:

Single-transgene, transgenic tobacco plants overexpressing GPT or GS1 were generated as described in Examples 3 and 4, respectively. Several of fastest growing T₂ generation GPT transgenic plant lines were crossed with the fastest growing T3 generation GS1 transgenic plant lines using reciprocal crosses. The progeny were then selected on kanamycin containing M&S media as described in Example 3, and their growth, flowering and seed yields examined.

Tissue extractions for GPT and GS activities: GPT activity was extracted from fresh plant tissue after grinding in cold 100 mM Tris-HCl, pH 7.6, containing 1 mm ethylenediaminetetraacetic, 200 mM pyridoxal phosphate and 6 mM mercaptoethanol in a ratio of 3 ml per gram of tissue. The extract was clarified by centrifugation and used in the assay. GS activity was extracted from fresh plant tissue after grinding in cold 50 mM Imidazole, pH 7.5 containing 10 mM MgCl2, and 12.5 mM mercaptoethanol in a ratio of 3 ml per gram of tissue. The extract was clarified by centrifugation and used in the assay. GPT activity was assayed as described in Calderon and Mora, 1985, Journal Bacteriology 161:807-809. GS activity was measured as described in Shapiro and Stadtmann, 1970, Methods in Enzymology 17A: 910-922. Both assays involve an incubation with substrates and cofactor at the proper pH. Detection was by HPLC.

### Results:

The results are presented in two ways. First, specific growth characteristics are tabulated in Tables IV.A and IV.B (biomass, seed yields, growth rate, GS activity, GPT activity, 2-oxoglutaramate activity, etc). Second, photographs of progeny plants and their leaves are shown in comparison to single-transgene and wild type plants and leaves are presented in FIG. 5 and FIG. 6, which show much larger whole plants, larger leaves, and earlier and/or more abundant flowering in comparison to the parental single-transgene plants and wild type control plants.

Referring to Table IV.A, double-transgene progeny plants form these crosses showed tremendous increases total biomass (fresh weight), with fresh weights ranging from 45-89 grams per individual progeny plant, compared to a range of only 19-24 grams per individual wild type plant, representing on average, about a two- to three-fold increase over wild type plants, and representing at the high end, an astounding four-fold increase in biomass over wild type plants. Taking the 24 individual double-transgene progeny plants evaluated, the average individual plant biomass was about 2.75 times that of the average wild type control plant. Four of the progeny lines showed approximately 2.5 fold greater average per plant fresh weights, while two lines showed over three-fold greater fresh weights in comparison to wild type plants.

In comparison to the single-transgene parental lines, the double-transgene progeny plants also showed far more than an additive growth enhancement. Whereas GPT single-transgene lines show as much as about a 50% increase over wild type biomass, and GS1 single-transgene lines as much as a 66% increase, progeny plants averaged almost a 200% increase over wild type plants.

Similarly, the double transgene progeny plants flowered earlier and more prolifically than either the wild type or single transgene parental lines, and produced a far greater number of seed pods as well as total number of seeds per plant. Referring again to Table IV.A, on average, the double-transgene progeny produced over twice the number of seed pods produced by wild type plants, with two of the high producer plants generating over three times the number of seed pods compared to wild type. Total seed yield in progeny plants, measured on a per plant weight basis, ranged from about double to nearly quadruple the number produced in wild type plants.

TABLE IV.A

| | FRESH WEIGHT | SEED PODS | SEED YIELD | | GS ACTIVITY | |
|---|---|---|---|---|---|---|
| PLANT LINE | g/whole plant | #pods/plant | g/plant | LEAF | ROOT | L/R RATIO |
| Wild Type Tobacco | | | | | | |
| Wild type 1 | 18.73 | 26 | 0.967 | | | |
| Wild type 2 | 24.33 | 24 | 1.07 | | | |
| Wild type 3 | 23.6 | 32 | 0.9 | | | |
| Wild type 4 | 18.95 | 32 | 1.125 | | | |
| WT Average | 21.4025 | 28.5 | 1.0155 | 7.75 | 1.45 | 5.34 |
| Cross 1 | | | | | | |
| X1L1a x PA9-9ff | | | | | | |
| 1 | 59.21 | 62 | 2.7811 | | | |
| 2 | 65.71 | 56 | | | | |
| 3 | 55.36 | 72 | | | | |
| 4 | 46.8 | 56 | | | | |
| Cross 1 Average | 56.77 | 61.5 | | 14.98 | 1.05 | 14.27 |
| Compared to WT | +265% | +216% | +274% | +193% | −28% | +267% |
| Cross 2 | | | | | | |
| PA9-2 x L9 | | | | | | |
| 1 | 70.83 | 61 | 1.76 | | | |
| 2 | 49.17 | 58 | 3.12 | | | |
| 3 | 50.23 | 90 NA | | | | |
| 4 | 45.77 | | | | | |
| Cross 2 Average | 54 | 58.3 | 2.44 | 16.32 | 1.81 | 9.02 |
| Compared to WT | +252% | +205% | +240% | +211% | +125% | +169% |
| Cross 3 | | | | | | |
| PA9-9ff xL1a | | | | | | |
| 1 | 89.1 | 77 | 3.687 | | | |
| 2 | 78.18 | | | | | |
| 3 | 58.34 | | | | | |
| 4 | 61.79 | | | | | |
| Cross 3 Average | 71.85 | 77 (one plant) | 3.678 (one plant) | 15.92 | 1.38 | 11.54 |
| Compared to WT | +336% | +270% | +362% | +205% | −5% | +216% |
| Cross 5 | | | | | | |
| PA9-10aa x L1a | | | | | | |
| 1 | 65.34 | 45 | 2.947 | | | |
| 2 | 53.28 | 64 | 3.3314 | | | |
| 3 | 49.85 | 42 | 1.5667 | | | |
| 4 | 44.63 | 42 | 2.5013 | | | |
| Cross 5 Average | 53.275 | 48.25 | 2.86928 | 13.03 | 1.8 | 7.24 |
| Compared to WT | +244% | +169% | +283% | +168% | | |
| Cross 6 | | | | | | |
| PA9-17b x L1a | | | | | | |
| 1 | 56.7 | 64 | 2.492 | | | |
| 2 | 55.05 | 66 | 2.162 | | | |
| 3 | 51.51 | 59 | 1.8572 | | | |
| 4 | 45.38 | 72 | 4.742 | | | |
| Cross 6 Average | 52.16 | 65.25 | 2.8133 | 14.114.7 | 1.1.1124 | 13.29 |
| Compared to WT | +244% | +229% | +277% | 52 | | |
| Cross 7 | | | | | | |
| PA9-20aa x L1b | | | | | | |
| 1 | 76.26 | 67 | 2.0535 | | | |
| 2 | 66.27 | 42 | 1.505 | | | |
| 3 | 72.26 | 72 | 2.3914 | | | |
| 4 | 63.91 | 91 | 2.87 | | | |
| Cross 7 Average | 69.675 | 68 | 2.204975 | 14.12 | 1.24 | 11.39 |
| Compared to WT | +326% | +239% | +217% | | | |
| Control PA9-9ff | | | | | | |
| 1 | 32.18 | N/A | | | | |
| 2 | 32.64 | N/A | | | | |
| 3 | 34.67 | N/A | | | | |
| 4 | 25.18 | N/A | | | | |
| Average | 31.17 | N/A | | 11.57 | 1.14 | 10.15 |
| Compared to WT | +148% | | | | | |
| Control GS L1a | | | | | | |
| 1 | 41.74 | N/A | | | | |
| 2 | 36.24 | N/A | | | | |
| 3 | 33.8 | N/A | | | | |
| 4 | 30.48 | N/A | | | | |
| Average | 35.57 | N/A | | 13.15 | 1.23 | 10.69 |
| Compared to WT | +166% | | | | | |

Table IV.B shows growth rate, biomass and yield, and biochemical characteristics of Line XX (Line 3 further selfed) compared to the single transgene line expressing GS1 and wild type control tobacco. All parameters are greatly increased in the double transgenic plant (Line XX). Notably, 2-oxoglutaramate activity was almost 17-fold higher, and seed yield and foliar biomass was three-fold higher, in Line XX plants versus control plants.

TABLE IV.B

| | Specific | | | | GS | GPT | | |
|---|---|---|---|---|---|---|---|---|
| | Growth | Foliar | Fruit/ | | Activity | Activity | 2- | Trans |
| Plant | Rate | Biomass | Flowers/ | Seed | umol/ | nmol/h/ | oxoglutaramate | Gene |
| Type | mg/g/d | FWt, g | Buds | Yield g | min/gFWt | gFWt | nmol/gFWt | Assay |
| Wildtype, | 228 | 21.40 | 28.5 | 1.02 | 7.75 | 16.9 | 68.9 | No |
| avg | | | | | | | | |
| Line 1 GS | 269 | 35.57 | NM | NM | 11.6 | NM | 414 | Yes |
| Line XX | 339 | 59.71 | 62.9 | 2.94 | 16.3 | 243.9 | 1,153.6 | Yes |

NM Not Measured

### Example 8

### Generation of Double Transgenic Pepper Plants Carrying GS1 and GPT Transgenes

In this example, Big Jim chili pepper plants (New Mexico varietal) were transformed with the Arabidopsis GPT full length coding sequence of SEQ ID NO: 1 under the control of the CMV 35S promoter, and the Arabidopsis GS1 coding sequence included in SEQ ID NO: 6 under the control of the RuBisCo promoter, using Agrobacterium-mediated transfer to seed pods. After 3 days, seeds were harvested and used to generate TO plants and screened for transformants. The resulting double-transgenic plants showed higher pod yields, faster growth rates, and greater biomass yields in comparison to the control plants.

### Materials and Methods:

Solanaceae Capisicum Pepper plants (“Big Jim” varietal) were transformed with the Arabidopsis GPT full length coding sequence of SEQ ID NO: 1 under the control of the CMV 35S promoter within the expression vector pMON (see Example 3), and the Arabidopsis GS1 coding sequence included in SEQ ID NO: 6 under the control of the RuBisCo promoter within the expression vector pCambia 1201 (Tomato rubisco rbcS3C promoter: Kyozulka et al., 1993, Plant Physiol. 103: 991-1000; SEQ ID NO: 22; vector construct of SEQ ID NO: 6), using Agrobacterium-mediated transfer to seed pods.

For this and all subsequent examples, the Cambia 1201 or 1305.1 vectors were constructed according to standard cloning methods (Sambrook et al., 1989, supra, Saiki et al., 1988, Science 239: 487-491). The vector is supplied with a 35S CaMV promoter; that promoter was replaced with RcbS-3C promoter from tomato to control the expression of the target gene. The Cambia 1201 vectors contain bacterial chlorophenicol and plant hygromycin resistance selectable marker genes. The Cambia 1305.1 vectors contain bacterial chlorophenicol and hygromycin resistance selectable marker genes.

The transgene expression vectors pMON (GPT transgene) and pCambia 1201 (GS transgene) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of either streptamycin for pMON constructs or chloroamphenicol for the Cambia constructs. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Pepper plants were then transformed with a mixture of the resulting Agrobacterium cell suspensions using a transformation protocol in which the Agrobacterium is injected directly into the seed cavity of developing pods. Briefly, developing pods were injected with the 200 ml mixture in order to inoculate immature seeds with the Agrobacteria essentially as described (Wang and Waterhouse, 1997, Plant Mol. Biol. Reporter 15: 209-215). In order to induce Agrobacteria virulence and improve transformation efficiencies, 10 μg/ml acetosyringonone was added to the Agrobacteria cultures prior to pod inoculations (see, Sheikholeslam and Weeks, 1986, Plant Mol. Biol. 8: 291-298).

Using a syringe, pods were injected with a liberal quantity of the Agrobacterium vector mixture, and left to incubate for about 3 days. Seeds were then harvested and germinated, and developing plants observed for phenotypic characteristics including growth and antibiotic resistance. Plants carrying the transgenes were green, whereas untransformed plants showed signs of chlorosis in leaf tips. Vigorous growing transformants were further cultivated and compared to wild type pepper plants grown under identical conditions.

### Results:

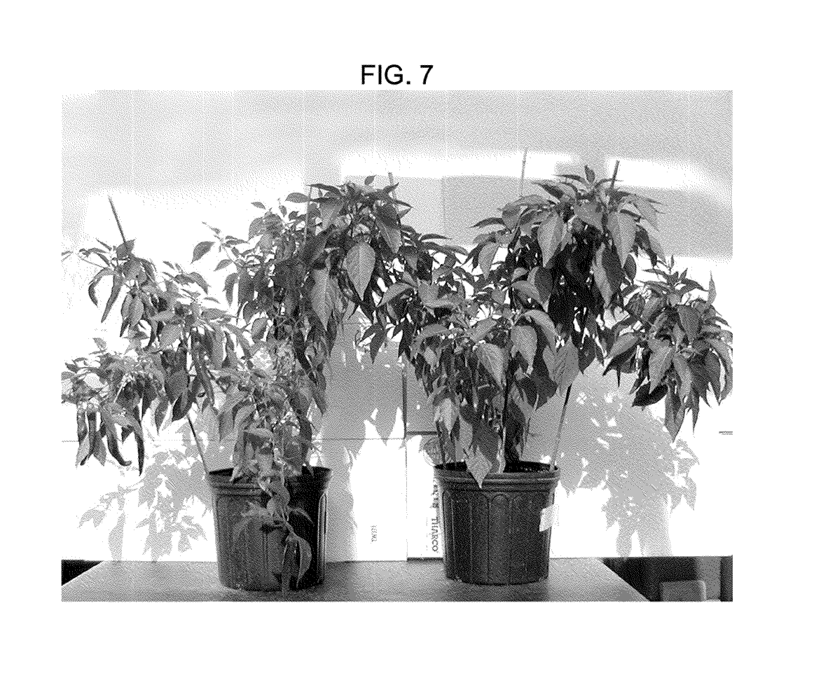

The results are presented in FIG. 7 and Table V. FIG. 7 shows a photograph of a GPT+GS double transgenic pepper plant compared to a control plant grown for the same time under identical conditions. This photograph shows tremendous pepper yield in the transgenic line compared to the control plant.

Table V presents biomass yield and GS activity, as well as transgene genotyping, in the transgenic lines compared to the wild type control. Referring to Table V, double-transgene progeny plants showed tremendous increases total biomass (fresh weight), with fresh weights, ranging from 393-662 grams per individual transgenic plant, compared to an average of 328 grams per wild type plant. Transgenic line A5 produced more than twice the total biomass of the controls. Moreover, pepper yields in the transgenic lines were greatly improved over wild type plants, and were 50% greater than control plants (on average). Notably, one of the transgene lines produced twice as many peppers as the control plant average.

TABLE V
TRANSGENIC PEPPER GROWTH/BIOMASS
AND REPRODUCTION

| | Biomass, | Yield | GS activity | Transgene |
|---|---|---|---|---|
| | Foliar Fresh | Peppers, g | Umoles/min/ | Presence |
| Plant type | Wt, g | DWt | gFWt | Assay |
| Wildtype, avg | 328.2 | 83.7 | 1.09 | Negative |
| Line A2 | 457.3 | 184.2 | 1.57 | GPT - Yes |
| Line A5 | 661.7 | 148.1 | 1.8 | GPT - Yes |
| Line B1 | 493.4 | 141.0 | 1.3 | GPT - Yes |
| Line B4 | 393.1 | 136.0 | 1.6 | GPT - Yes |
| Line C1 | 509.4 | 152.9 | 1.55 | GPT - Yes |

FWt Fresh Weight;
DWt Dry Weight

### Example 9

### Generation of Double Transgenic Bean Plants Carrying Arabidopsis GS1 and GPT Transgenes

In this example, yellow wax bean plants (Phaseolus vulgaris) were transformed with the Arabidopsis GPT full length coding sequence of SEQ ID NO: 1 under the control of the CMV 35S promoter within the expression vector pCambia 1201, and the Arabidopsis GS1 coding sequence included in SEQ ID NO: 6 under the control of the RuBisCo promoter within the expression vector pCambia 1201, using Agrobacterium-mediated transfer into flowers.

### Materials and Methods:

The transgene expression vectors pCambia 1201-GPT (including construct of SEQ ID NO: 27) and pCambia 1201-GS (including construct of SEQ ID NO: 6) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of chloroamphenicol. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Bean plants were then transformed with a mixture of the resulting Agrobacterium cell suspensions using a transformation protocol in which the Agrobacteria is injected directly into the flower structure (Yasseem, 2009, Plant Mol. Biol. Reporter 27: 20-28). In order to induce Agrobacteria virulence and improve transformation efficiencies, 10 μg/ml acetosyringonone was added to the Agrobacteria cultures prior to flower inoculation. Briefly, once flowers bloomed, the outer structure encapsulating the reproductive organs was gently opened with forceps in order to permit the introduction of the Agrobacteria mixture, which was added to the flower structure sufficient to flood the anthers.

Plants were grown until bean pods developed, and seeds were harvested and used to generate transgenic plants. Transgenic plants were then grown together with control bean plants under identical conditions, photographed and phenotypically characterized. Growth rates were measured for both transgenic and control plants. In this and all examples, Glutamine synthetase (GS) activity was assayed according to the methods in Shapiro and Stadtmann, 1970, Methods in Enzymology 17A: 910-922; and, Glutamine phenylpyruvate transaminase (GPT) activity was assayed according to the methods in Calderon et al., 1985, J. Bacteriol. 161: 807-809. See details in Example 7, Methods, supra.

### Results:

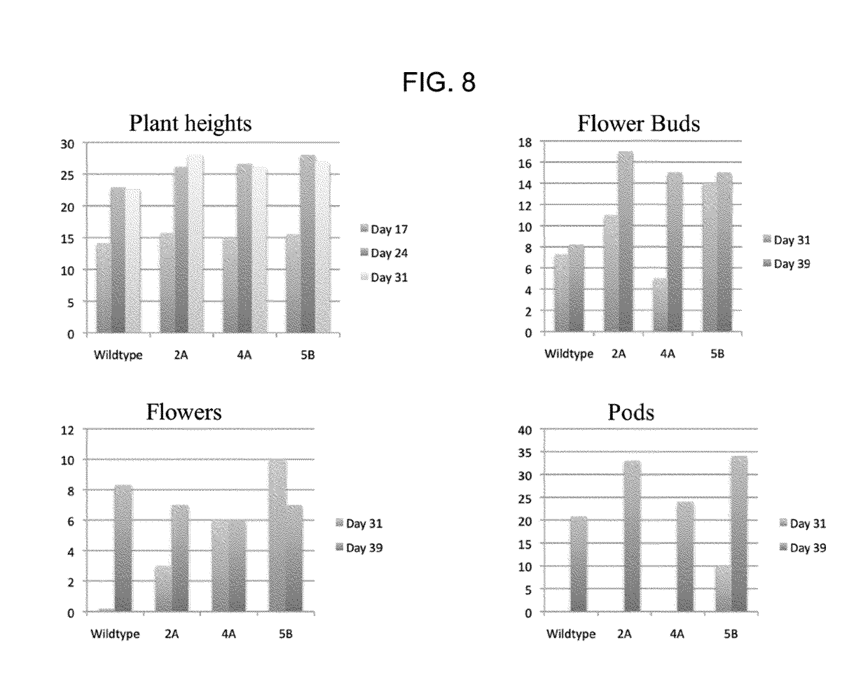

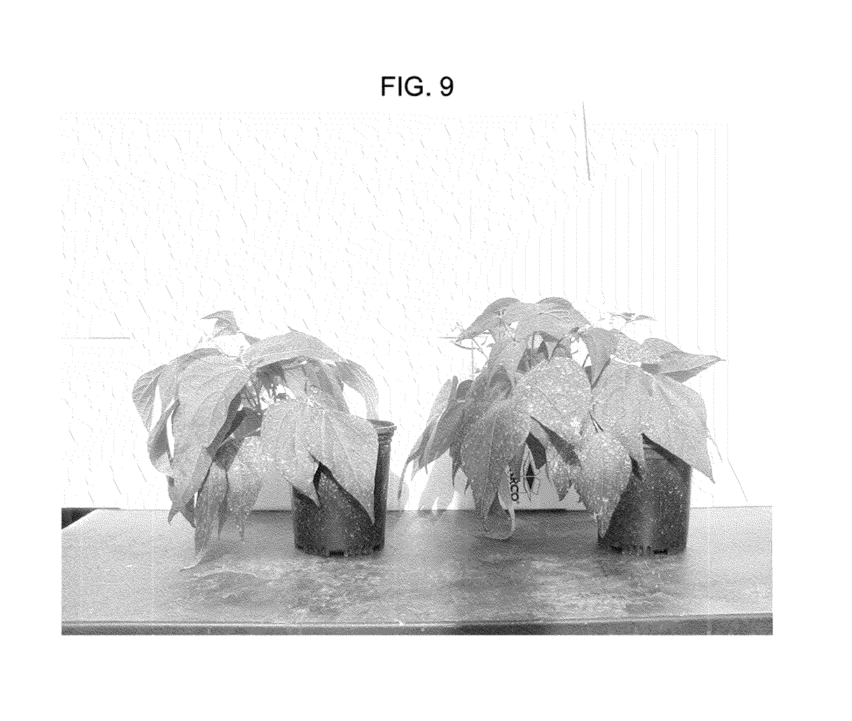

The results are presented in FIG. 8, FIG. 9 and Table VI.

FIG. 8 shows GPT+GS transgenic bean line A growth rate data relative to control plants, including plant heights on various days into cultivation, as well as numbers of flower buds, flowers, and bean pods. These data show that the GPT+GS double transgenic bean plants outgrew their counterpart control plants. The transgenic plants grew taller, flowered earlier and produced more flower buds and flowers, and developed bean pods and produced more bean pods that the wild type control plants.

TABLE VI
TRANSGENIC BEANS LINE A

| | | | GS Activity | |
|---|---|---|---|---|
| | Bean Pod | GPT Activity | umoles/min/ | Antibiotic |
| Plant Type | Yield FWt, g | nmoles/h/gFWt | gFWt | Resistance |
| Wildtype, avg | 126.6 | 101.9 | 25.2 | Negative |
| 2A | 211.5 | NM | NM | + |
| 4A | 207.7 | NM | NM | + |
| 5B | 205.7 | 984.7 | 101.3 | + |

WT Wildtype;
FWt Fresh Weight;
NM Not Measured

Table VI presents bean pod yield, GPT and GS activity, as well as antibiotic resistance status, in the transgenic lines compared to the wild type control (average of several robust control plants; control plants that did not grow well were excluded from the analyses). Referring to Table VI, double-transgene progeny plants showed substantial bean pod biomass increases (fresh pod weight) in comparison to the control plants, with bean pod biomass yields consistently above 200 grams per individual transgenic plant, compared to an average of 127 grams per wild type plant, representing an over 60% increase in pod yield in the double transgene lines relative to control plant(s).

Lastly, FIG. 9 shows a photograph of a GPT+GS double transgenic bean plant compared to a control plant grown for the same time under identical conditions, showing increased growth in the transgenic plant.

### Example 10

### Generation of Double Transgenic Bean Plants Carrying Arabidopsis GS1 and Grape GPT Transgenes

In this example, yellow wax bean plants (Phaseolus vulgaris) were transformed with the Grape GPT full length coding sequence included in SEQ ID NO: 8 under the control of the RuBisCo promoter within the expression vector pCambia 1305.1, and the Arabidopsis GS1 coding sequence included in SEQ ID NO: 6 under the control of the RuBisCo promoter within the expression vector pCambia 1201, using Agrobacterium-mediated transfer into developing pods.

### Materials and Methods:

The transgene expression vectors pCambia 1201-GPT (grape) (including construct of SEQ ID NO: 8) and pCambia 1201-GS (including construct of SEQ ID NO: 6) were transferred to separate Agrobacterium tumefaciens strain LBA4404 cultures using a standard electroporation method (McCormac et al., 1998, Molecular Biotechnology 9:155-159). Transformed Agrobacterium were selected on media containing 50 μg/ml of chloroamphenicol. Transformed Agrobacterium cells were grown in LB culture media containing 25 μg/ml of antibiotic for 36 hours. At the end of the 36 hr growth period cells were collected by centrifugation and cells from each transformation were resuspended in 100 ml LB broth without antibiotic.

Bean plants were then transformed with a mixture of the resulting Agrobacterium cell suspensions using a transformation protocol in which the Agrobacteria is injected directly into the flower structure. In order to induce Agrobacteria virulence and improve transformation efficiencies, 10 μg/ml acetosyringonone was added to the Agrobacteria cultures prior to flower inoculation. Briefly, once flowers bloomed, the outer structure encapsulating the reproductive organs was gently opened with forceps in order to permit the introduction of the Agrobacteria mixture, which was added to the flower structure sufficient to flood the anthers.

Plants were grown until bean pods developed, and seeds were harvested and used to generate transgenic plants. Transgenic plants were then grown together with control bean plants under identical conditions, photographed and phenotypically characterized. Growth rates were measured for both transgenic and control plants.

### Results:

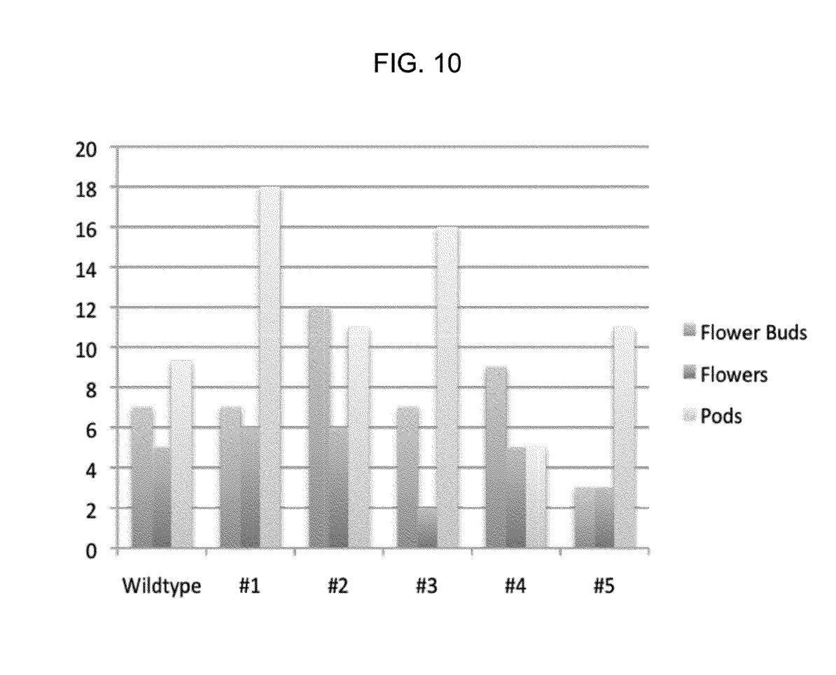

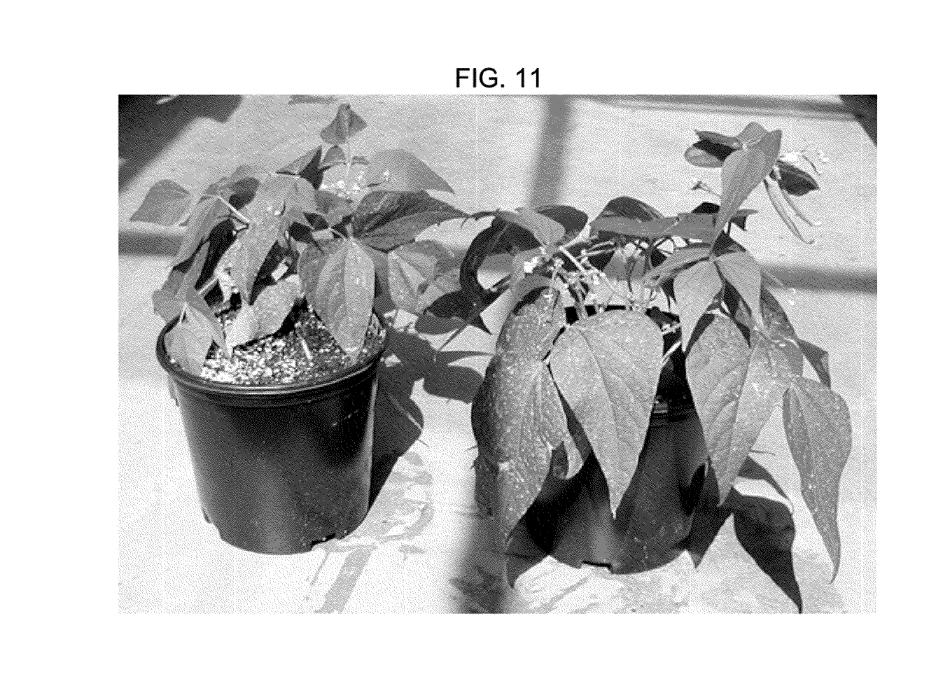

The results are presented in FIG. 10, FIG. 11 and Table VII.

FIG. 10 shows GPT+GS transgenic bean line G growth rate data relative to control plants, specifically including numbers of flower buds, flowers, and bean pods. These data show that the GPT+GS double transgenic bean plants outgrew their counterpart control plants. Notably, the transgenic plants produced substantially more bean pods that the wild type control plants.

TABLE VII
TRANSGENIC BEANS LINE G: POD YIELDS

| Plant Type | Bean Pod Yield FWt, g | Antibiotic Resistance |
|---|---|---|
| Wild type, avg | 157.9 | Negative |
| G1 | 200.5 | + |
| G2 | 178.3 | + |

WT Wildtype;
FWt Fresh Weight;
NM Not Measured

Table VII presents bean pod yield and antibiotic resistance status, in the transgenic lines compared to the wild type control (average of several robust control plants; control plants that did not grow well were excluded from the analyses). Referring to Table VII, double-transgene progeny plants showed substantial bean pod biomass increases (fresh pod weight) in comparison to the control plants, with bean pod biomass yields of 200.5 (line G1) and 178 grams (line G2) per individual transgenic plant, compared to an average of 158 grams per individual wild type plant, representing approximately a 27% increase in pod yield in the double transgene lines relative to control plants.

Lastly, FIG. 11 shows a photograph of a GPT+GS double transgenic bean plant compared to a control plant grown for the same time under identical conditions. The transgenic plant shows substantially increased size and biomass, larger leaves and a more mature flowering compared to the control plant.

### Example 11

### Generation of Double Transgenic Cowpea Plants Carrying Arabidopsis GS1 and GPT Transgenes

In this example, common Cowpea plants were transformed with the Arabidopsis GPT full length coding sequence of SEQ ID NO: 1 under the control of the CMV 35S promoter within the expression vector pMON, and the Arabidopsis GS1 coding sequence included in SEQ ID NO: 6 under the control of the RuBisCo promoter within the expression vector pCambia 1201, using Agrobacterium-mediated transfer into flowers. Materials and methods were as in Example 9, supra.

### Results:

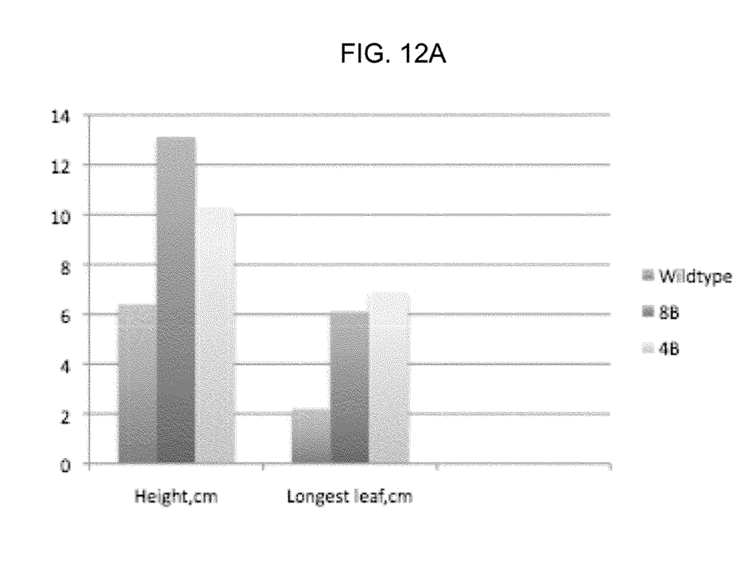

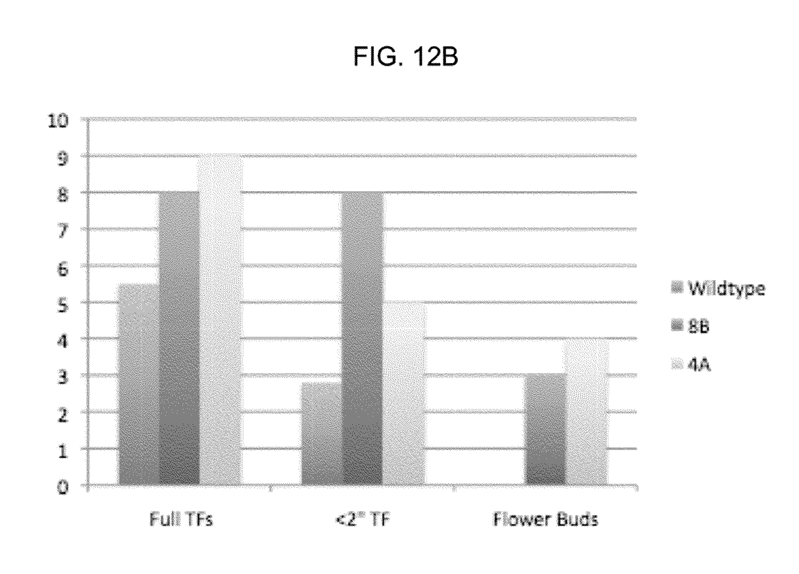

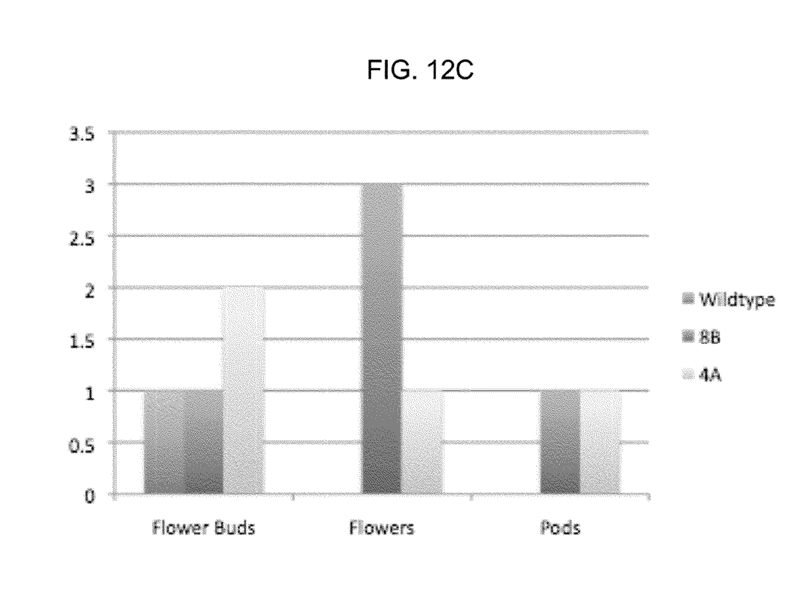

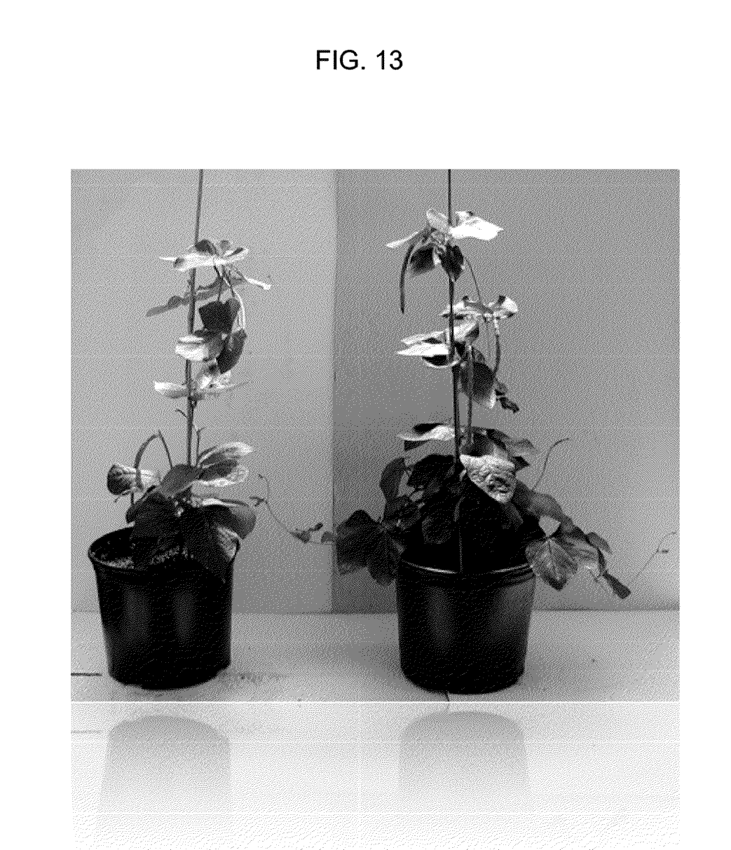

The results are presented in FIGS. 12 and 13, and Table VI. FIG. 12 shows relative growth rates for the GPT+GS transgenic Cowpea line A and wild type control Cowpea at several intervals during cultivation, including (FIG. 12A) height and longest leaf measurements, (FIG. 12B) trifolate leafs and flower buds, and (FIG. 12C) flowers, flower buds and pea pods. These data show that the GPT+GS double transgenic Cowpea plants outgrew their counterpart control plants. The transgenic plants grew faster and taller, had longer leaves, and set flowers and pods sooner than wild type control plants.

TABLE VIII
TRANSGENIC COWPEA LINE A

| | Pea Pod | | | |
|---|---|---|---|---|
| | Yield, | GPT Activity | GS Activity | Antibiotic |
| Plant Type | FWt, g | nmoles/h/gFWt | umol/min/gFWt | Resistance |
| Wildtype, avg | 74.7 | 44.4 | 28.3 | Negative |
| 4A | 112.8 | NM | 41.3 | + |
| 8B | 113.8 | 736.2 | 54.9 | + |

WT Wildtype;
FWt Fresh Weight;
NM Not Measured

Table VIII presents pea pod yield, GPT and GS activity, as well as antibiotic resistance status, in the transgenic lines compared to the wild type control (average of several robust control plants; control plants that did not grow well were excluded from the analyses). Referring to Table VIII, double-transgene progeny plants showed substantial pea pod biomass increases (fresh pod weight) in comparison to the control plants, with average transgenic plant pea pod biomass yields nearly 52% greater than the yields measured in control plant(s).

Lastly, FIG. 13 shows a photograph of a GPT+GS double transgenic bean plant compared to a control plant grown for the same time under identical conditions, showing increased biomass and pod yield in the transgenic plant relative to the wild type control plant.

### Example 12

### Generation of Double Transgenic Cowpea Plants Carrying Arabidopsis GS1 and Grape GPT Transgenes

In this example, common Cowpea plants were transformed with the Grape GPT full length coding sequence included in SEQ ID NO: 8 under the control of the RuBisCo promoter within the expression vector pCambia 1305.1 (including construct of SEQ ID NO: 8), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer into flowers. Materials and methods were as in Example 11, supra.

### Results:

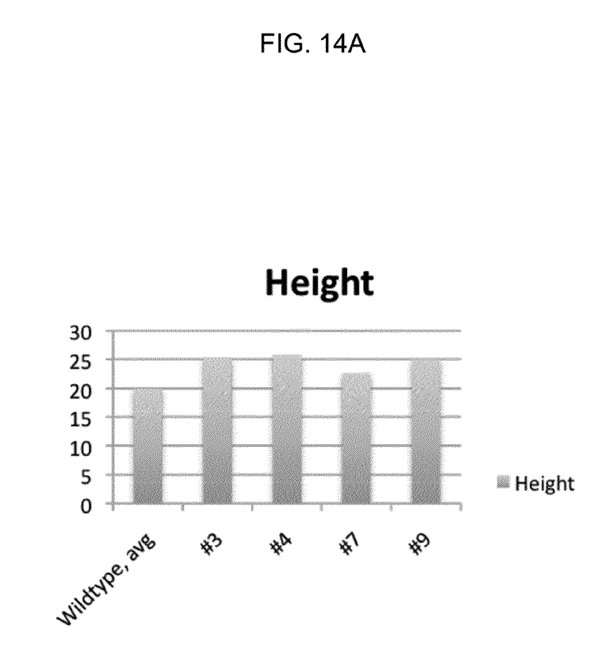

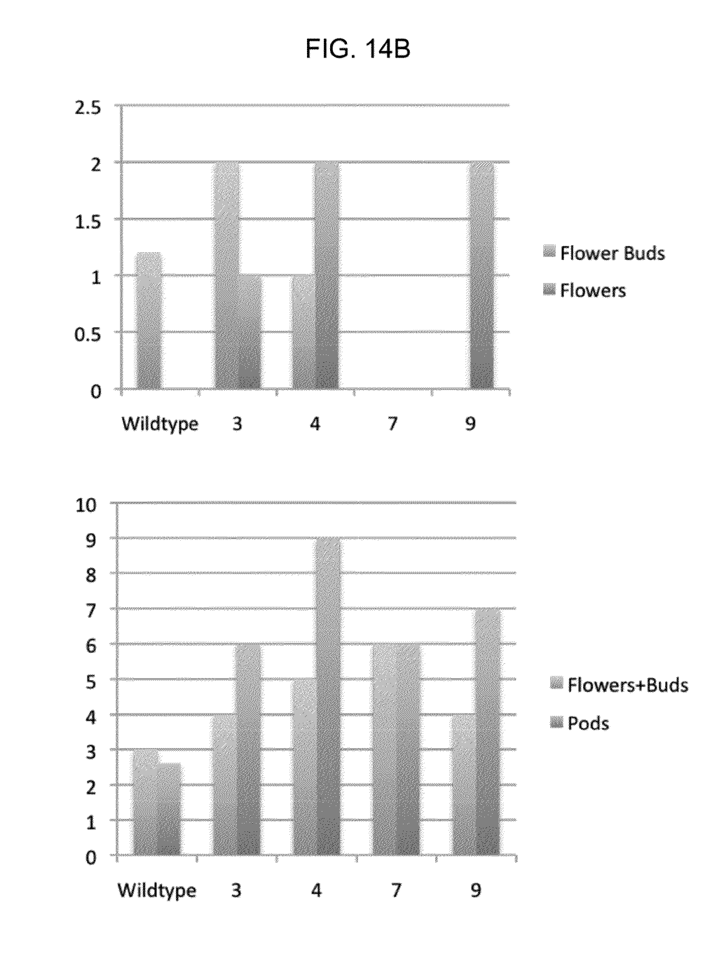

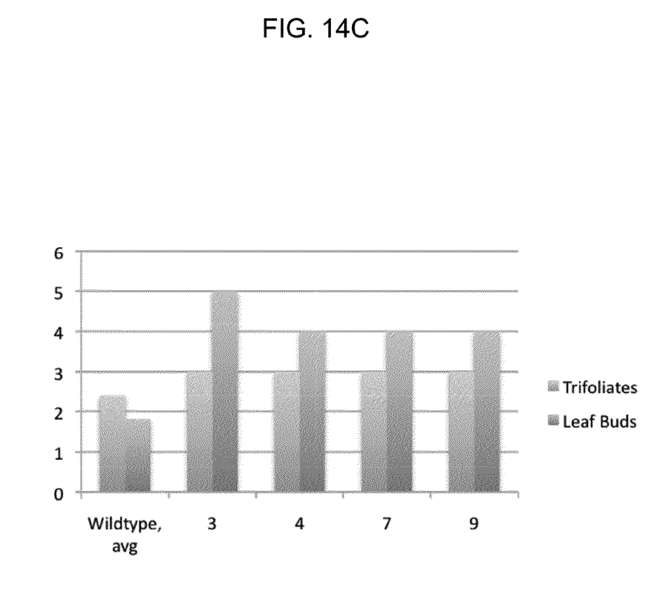

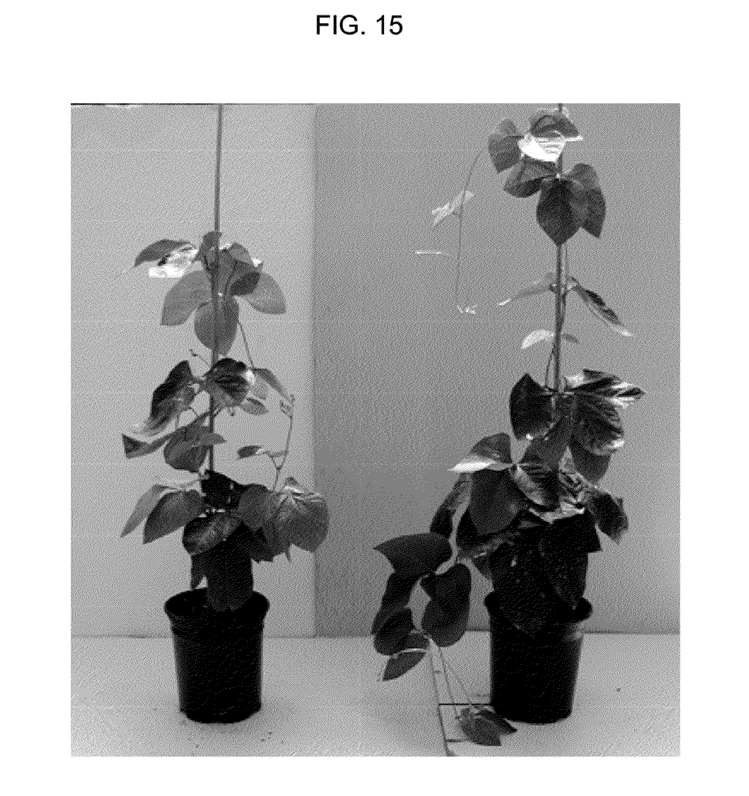

The results are presented in FIGS. 14 and 15, and Table IX.

FIG. 14 shows relative growth rates for the GPT+GS transgenic Cowpea line G and wild type control Cowpea. These data show that the transgenic plants are consistently higher (FIG. 14A), produce substantially more flowers, flower buds and pea pods (FIG. 14B), and develop trifolates and leaf buds faster (FIG. 14C).

TABLE IX
TRANSGENIC COWPEA LINE G

| | Pod Yield, | GPT Activity | GS Activity | Antibiotic |
|---|---|---|---|---|
| Plant Type | FWt, g | nmoles/h/gFWT | umol/min/gFWt | Resistance |
| Wildtype, | 59.7 | 44.4 | 26.7 | Negative |
| avg | | | | |
| G9 | 102.0 | 555.6 | 34.5 | + |

WT Wildtype;
FWt Fresh Weight;
NM Not Measured

Table IX presents pea pod yield, GPT and GS activity, as well as antibiotic resistance status, in the transgenic lines compared to the wild type control (average of several robust control plants; control plants that did not grow well were excluded from the analyses). Referring to Table IX, double-transgene progeny plants showed substantial pea pod biomass increases (fresh pod weight) in comparison to the control plants, with average pea pod biomass yields 70% greater in the transgenic plants compared to control plant(s).

Lastly, FIG. 15 shows a photograph of a GPT+GS double transgenic pea plant compared to a control plant grown for the same time under identical conditions, showing increased height, biomass and leaf size in the transgenic plant relative to the wild type control plant.

### Example 13

### Generation of Double Transgenic Alfalfa Plants Carrying Arabidopsis GS1 and GPT Transgenes

In this example, Alfalfa plants (Medicago sativa, var Ladak) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer into seedling plants. Agrobacterium vectors and mixtures were prepared for seedling inoculations as described in Example 11, supra.

### Seedling Inoculations:

When Alfalfa seedlings were still less than about ½ inch tall, they were soaked in paper toweling that had been flooded with the Agrobacteria mixture containing both transgene constructs. The seedlings were left in the paper toweling for two to three days, removed and then planted in potting soil. Resulting TO and control plants were then grown for the first 30 days in a growth chamber, thereafter cultivated in a greenhouse, and then harvested 42 days after sprouting. At this point, only the transgenic Alfalfa line displayed flowers, as the wild type plants only displayed immature flower buds. The plants were characterized as to flowering status and total biomass.

### Results:

The results are presented in Table X. The data shows that the transgenic Alfalfa plants grew faster, flowered sooner, and yielded on average about a 62% biomass increase relative to the control plants.

TABLE X
TRANSGENIC ALFALFA VS. CONTROL

| Plant Type | Biomass at Sacrifice, g | Flowering Stage |
|---|---|---|
| Wildtype, avg | 6.03 | Small defined buds |
| | | No buds swelling. |
| | | No flowers |
| Transgene #5 | 10.38 | 4 Open flowers |
| Transgene # 11 | 9.03 | Flower buds swelling |
| Transgene #13 | 9.95 | Flower buds swelling |

### Example 14

### Generation of Double Transgenic Cantaloupe Plants Carrying Arabidopsis GS1 and GPT Transgenes

In this example, Cantaloupe plants (Cucumis melo var common) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer via injection into developing melons. Agrobacterium vectors and mixtures were prepared for intra-melon inoculations as described in Example 8, supra. Inoculations into developing melons were carried out essentially as described in Example 8. The plants were characterized as to flowering status and total biomass relative to control melon plants grown under identical conditions.

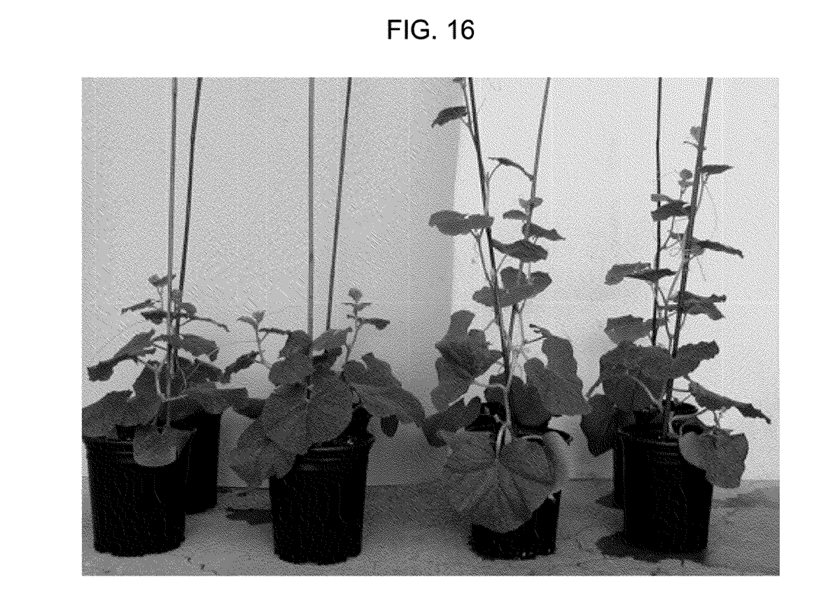

The results are presented in FIG. 16 and Table XI. Referring to Table XI, the transgenic plants showed substantial foliar plant biomass increases in comparison to the control plants, with an average increase in biomass of 63%. Moreover, a tremendous increase in flower and flower bud yields was observed in all five transgenic lines. Control plants displayed no flowers and only 5 buds at sacrifice, on average. In sharp contrast, the transgenic plants displayed between 2 and 5 flowers per plant, and between 21 and 30 flower buds, per plant, indicating a substantially higher growth rate and flower yield. Increased flower yield would be expected to translate into correspondingly higher melon yields in the transgenic plants. Referring to FIG. 16 (a photograph comparing transgenic Cantaloupe plants to control Cantaloupe plants), the transgenic Cantaloupe plants show dramatically increased height, overall biomass and flowering status relative to the control plants.

TABLE XI
TRANGENIC CANTALOUPE VERSUS CONTROL

| | Biomass | Flowers/Flower | Antibiotic |
|---|---|---|---|
| Plant Type | Foliar FWt, g | Buds at Sacrifice | Resistance |
| Wildtype, avg | 22.8 | 0/5 | Negative |
| Line 1 | 37.0 | 3/21 | + |
| Line 2 | 35.0 | 2/30 | + |
| Line 3 | 37.1 | 3/27 | + |
| Line 4 | 40.6 | 5/26 | + |
| Line 5 | 35.7 | 4/30 | + |

FWt Fresh Weight

### Example 15

### Generation of Double Transgenic Pumpkin Plants Carrying Arabidopsis GS1 and GPT Transgenes

In this example, common Pumpkin plants (Cucurbita maxima) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6), using Agrobacterium-mediated transfer via injection into developing pumpkins, essentially as described in Example 14, supra. The transgenic and control pumpkin plants were grown under identical conditions until the emergence of flower buds in the control plants, then all plants were characterized as to flowering status and total biomass.

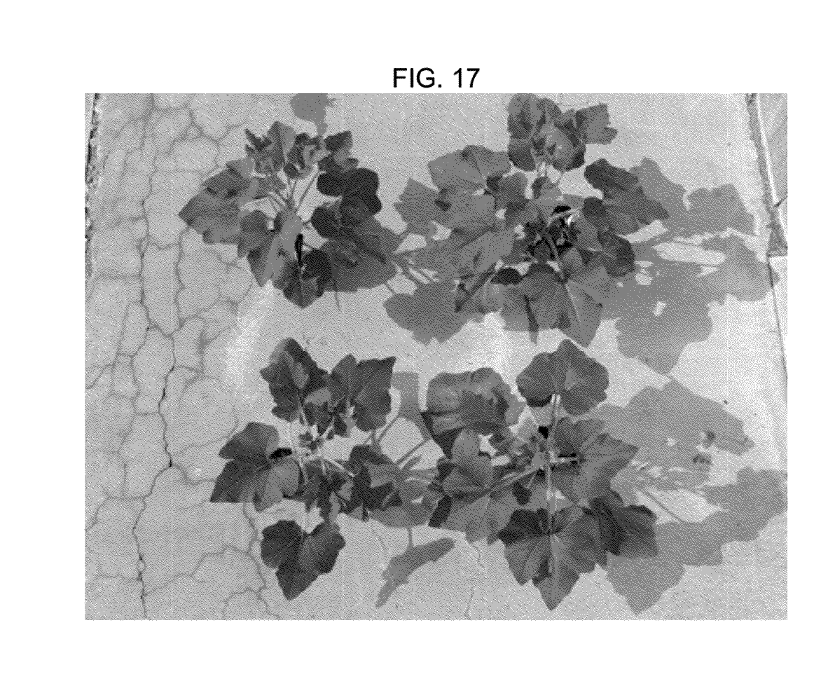

The results are presented in FIG. 17 and Table XII. Referring to Table XII, the transgenic plants showed substantial foliar plant biomass increases in comparison to the control plants, with an increase in average biomass yield of 67% over control plants. Moreover, an increase in flower bud yields was observed in four of the five transgenic lines in comparison to control. Control plants displayed only 4 buds at sacrifice (average). In contrast, four transgenic plant lines displayed between 8 and 15 flowers buds per plant, representing a two- to nearly four-fold yield increase.

TABLE XII
TRANGENIC PUMPKIN VERSUS CONTROL

| | Biomass | Flower Buds at | Antibiotic |
|---|---|---|---|
| Plant Type | Foliar FWt, g | Sacrifice | Resistance |
| Wildtype, avg | 47.7 | 4.2 | Negative |
| Line 1 (Photo) | 82.3 | 8 | |
| Line 2 | 74.3 | 8 | + |
| Line 3 | 80.3 | 9 | + |
| Line 4 (Photo) | 77.8 | 4 | + |
| Line 5 | 84.5 | 15 | + |

FWt Fresh Weight;

Referring to FIG. 17 (a photograph comparing transgenic pumpkin plants to control plants), the transgenic pumpkin plants show substantially increased plant size, overall biomass and leaf sizes and numbers relative to the control plants.

### Example 16

### Generation of Double Transgenic Arabidopsis Plants Carrying Arabidopsis GS1 and GPT Transgenes

In this example, Arabidopsis thaliana plants were transformed with the truncated Arabidopsis GPT coding sequence of SEQ ID NO: 18 under the control of the CMV 35S promoter within the expression vector pMON316 (see Example 3, supra), and transgenic plants thereafter transformed with the Arabidopsis GS1 coding sequence included in SEQ ID NO: 6 under the control of the RuBisCo promoter within the expression vector pCambia 1201 (including construct of SEQ ID NO: 6), using Agrobacterium-mediated “floral dip” transfer as described (Harrison et al., 2006, Plant Methods 2:19-23; Clough and Bent, 1998, Plant J. 16:735-743). Agrobacterium vectors pMON316 carrying GPT and pCambia 1201 carrying GS1 were prepared as described in Examples 3 and 11, respectively.

Transformation of two different cultures of Agrobacterium with either a pMon 316+Arabidopsis GTP construct or with a Cambia 1201+Arabidopsis GS construct was done by electroporation using the method of Weigel and Glazebrook 2002. The transformed Agrobacterium were then grown under antibiotic selection, collected by centrifugation resuspended in LB broth with antibiotic and used in the floral dip of Arabidopsis inflorescence. Floral dipped Arabidopsis plants were taken to maturity and self-fertilized and seeds were collected. Seeds from twice dipped plants were first geminated on a media containing 20 ug/ml of kanamycin and by following regular selection procedures surviving seedlings were transferred to media containing 20 ug of hygromycin. Plants (3) surviving the selection process on both antibiotics were self-fertilized and seeds were collected. Seeds from the T1 generation were germinated on MS media containing 20 ug/ml of hygromycin and surviving seedlings were taken to maturity, self-fertilized and seeds collected. This seed population the T2 generation was then used for subsequent growth studies.

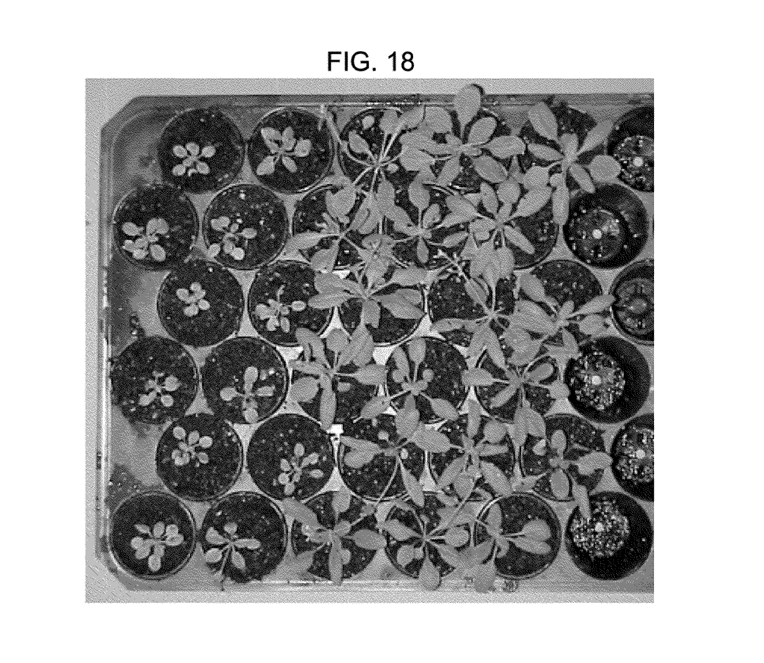

The results are presented in FIG. 18 and Table XIII. Referring to Table XIII, which shows data from 6 wild type and 6 transgenic Arabidopsis plants (averaged), the transgenic plants displayed increased levels of both GPT and GS activity. GPT activity was over twenty-fold higher than the control plants. Moreover, the transgenic plant fresh foliar weight average was well over four-fold that of the wild type control plant average. A photograph of young transgene Arabidopsis plants in comparison to wild type control Arabidopsis plants grown under identical conditions is shown in FIG. 18, and reveals a consistent and very significant growth/biomass increase in transgenic plants relative to the control plants.

TABLE XIII
TRANSGENIC ARABIDOPSIS VERSUS CONTROL

| | | GPT | GS Activity | |
|---|---|---|---|---|
| | Biomass, g | Activity | umol/ | Antibiotic |
| Plant type | Fresh foliar wt | nmol/h/gFWt | min/gFWt | Resistance |
| Wildtype, avg | 0.246 | 18.4 | 7.0 | Negative |
| Transgene | 1.106 | 395.6 | 18.2 | Positive |

### Example 17

### Generation of Transgenic Tomato Plants Carrying Arabidopsis GPT and GS1 Transgenes

In this example, tomato plants (Solanum lycopersicon, “Money Maker” variety) were transformed with the Arabidopsis GPT full length coding sequence of SEQ ID NO: 1 under the control of the CMV 35S promoter within the expression vector pMON316 (see Example 3, supra), and the Arabidopsis GS1 coding sequence included in SEQ ID NO: 6 under the control of the RuBisCo promoter within the expression vector pCambia 1201 (including construct of SEQ ID NO: 6). Single transgene (GPT) transgenic tomato plants were generated and grown to flowering essentially as described in Example 4. The Arabidopsis GS1 transgene was then introduced into the single-transgene TO plants using Agrobacterium-mediated transfer via injection directly into flowers (as described in Example 8). The transgenic and control tomato plants were grown under identical conditions and characterized as to growth phenotype characteristics. Resulting TO double-transgene plants were then grown to maturity, photographed along with control tomato plants, and phenotypically characterized.

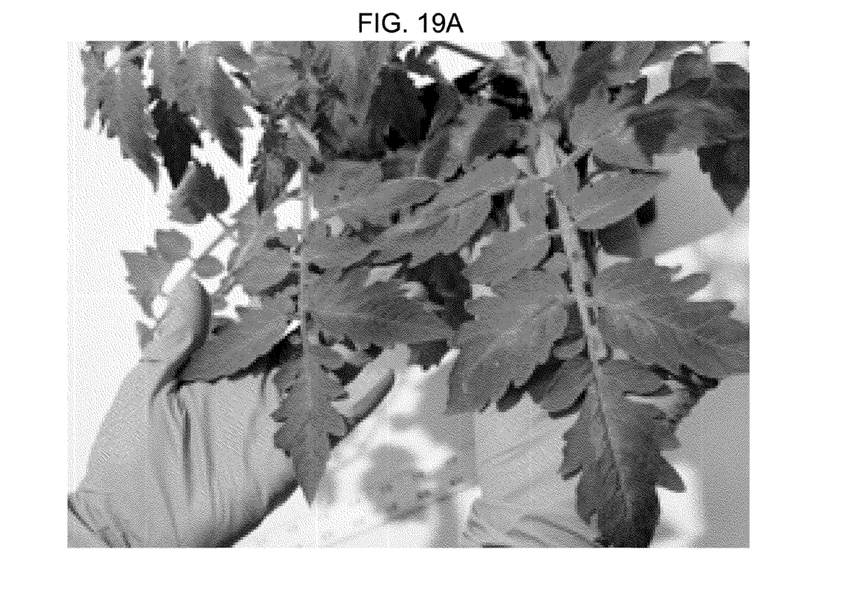

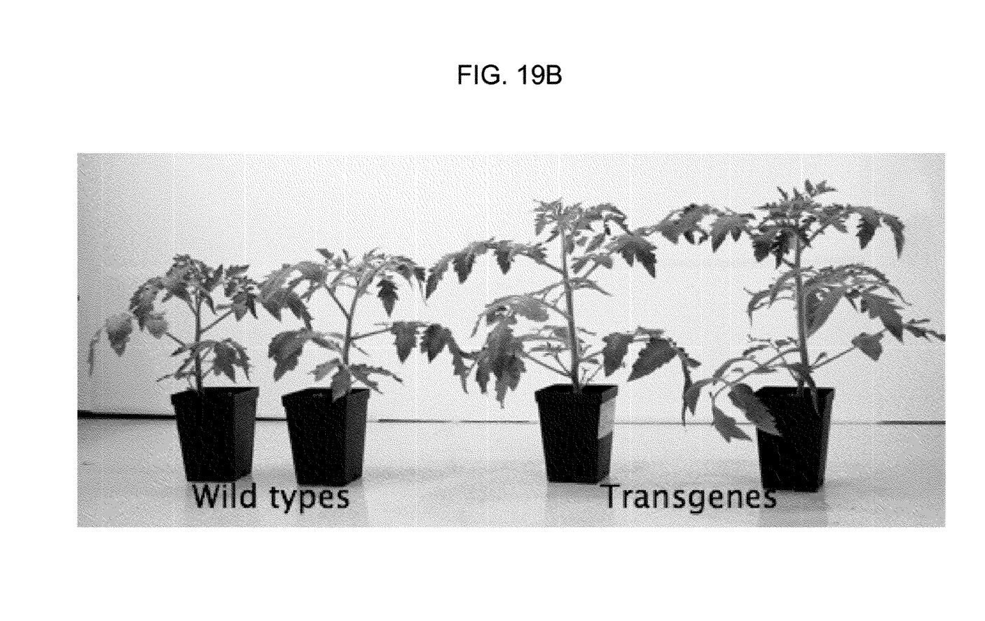

The results are presented in FIG. 19 and in Table X. Referring to Table XIX, double-transgene tomato plants showed substantial foliar plant biomass increases in comparison to the control plants, with an increase in average biomass yield of 45% over control. Moreover, as much as a 70% increase in tomato fruit yield was observed in the transgenic lines compared to control plants (e.g., 51 tomatoes harvested from Line 4C, versus and average of approximately 30 tomatoes from control plants). A much higher level of GPT activity was observed in the transgenic plants (e.g., line 4C displaying an approximately 32-fold higher GPT activity in comparison to the average GPT activity measured in control plants). GS activity was also higher in the transgenic plants relative to control plants (almost double in Line 4C).

With respect to growth phenotype, and referring to FIG. 19, the transgenic tomato plants displayed substantially larger leaves compared to control plants (FIG. 19A). In addition, it can be seen that the transgenic tomato plants were substantially larger, taller and of a greater overall biomass (see FIG. 19B).

TABLE XIX
TRANSGENIC TOMATO GROWTH AND REPRODUCTION

| | | Total | | | |
|---|---|---|---|---|---|
| | | Tomatoes | GPT | | |
| | Biomass | Harvested | Activity | GS Activity | Transgene |
| | Foliar | until | nmoles/h/ | umoles/min/ | Presence |
| Plant Type | FWt, g | Sacrifice | gFWt | gFWt | Assay |
| Wildtype, | 891 | 30.2 | 287 | 14.27 | Negative |
| avg | | | | | |
| Line 6C | 1288 | 43 | 9181 | 18.3 | + |
| Line 4C | 1146 | 51 | 1718 | 26.4 | + |

### Example 18

### Generation of Transgenic Camilena Plants Carrying Arabidopsis GPT and GS1 Transgenes

In this example, Camelina plants (Camelina sativa, Var MT 303) were transformed with the Arabidopsis GPT full length coding sequence of SEQ ID NO: 1 under the control of the RuBisCo promoter within the expression vector pCambia 1201, and the Arabidopsis GS1 coding sequence included in SEQ ID NO: 6 under the control of the RuBisCo promoter within the expression vector pCambia 1201, using Agrobacterium-mediated transfer into germinating seeds according to the method described in Chee et al., 1989, Plant Physiol. 91: 1212-1218. Agrobacterium vectors and mixtures were prepared for seed inoculations as described in Example 11, supra.

Transgenic and control Camelina plants were grown under identical conditions (30 days in a growth chamber and then moved to greenhouse cultivation) for 39 days, and characterized as to biomass, growth characteristics and flowering stage.

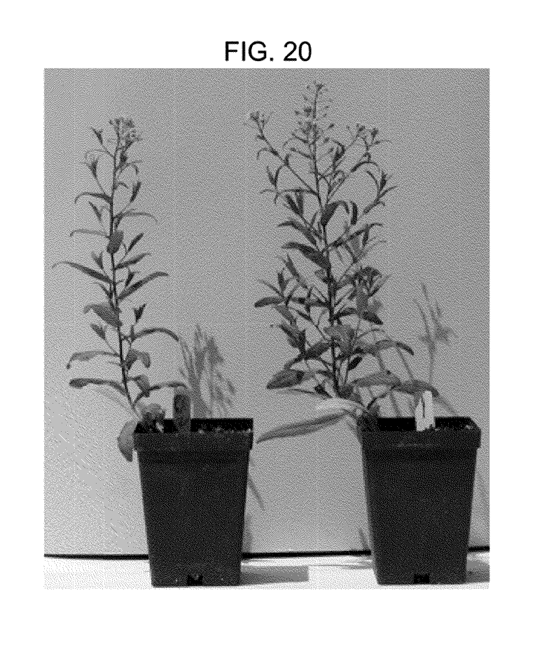

The results are presented in Table XX and FIG. 20. Referring to Table XX, it can be seen that total biomass in the transgenic plants was, on average, almost double control plant biomass. Canopy diameter was also significantly improved in the transgenic plants. FIG. 20 shows a photograph of transgenic Camelina compared to control. The transgenic plant is noticeably larger and displays more advanced flowering.

TABLE XX
TRANSGENIC CAMELINA VERSUS CONTROL

| | Height/Canopy | | |
|---|---|---|---|
| Plant Type | Diameter, inches | Biomass g | Flowering Stage |
| Wildtype, avg | 14/4 | 8.35 | Partial flowering |
| Transgene C-1 | 15.5/5 | 16.54 | Full flowering |
| Transgene C-3 | 14/7 | 14.80 | Initial flowering |

### Example 19

### Activity of Barley GPT Transgene in Planta

In this example, the putative coding sequence for Barley GPT was isolated and expressed from a transgene construct using an in planta transient expression assay. Biologically active recombinant Barley GPT was produced, and catalyzed the increased synthesis of 2-oxoglutaramate, as confirmed by HPLC.

The Barley (Hordeum vulgare) GPT coding sequence was determined and synthesized. The DNA sequence of the Barley GPT coding sequence used in this example is provided in SEQ ID NO: 14, and the encoded GPT protein amino acid sequence is presented in SEQ ID NO: 15.

The coding sequence for Barley GPT was inserted into the 1305.1 cambia vector, and transferred to Agrobacterium tumefaciens strain LBA404 using a standard electroporation method (McCormac et al., 1998, Molecular Biotechnology 9:155-159), followed by plating on LB plates containing hygromycin (50 micro gm/ml). Antibiotic resistant colonies of Agrobacterium were selected for analysis.

The transient tobacco leaf expression assay consisted of injecting a suspension of transformed Agrobacterium (1.5-2.0 OD 650) into rapidly growing tobacco leaves. Intradermal injections were made in a grid across the leaf surface to assure that a significant amount of the leaf surface would be exposed to the Agrobacterium. The plant was then allowed to grow for 3-5 days when the tissue was extracted as described for all other tissue extractions and the GPT activity measured.

GPT activity in the inoculated leaf tissue (1217 nanomoles/gFWt/h) was three-fold the level measured in the control plant leaf tissue (407 nanomoles/gFWt/h), indicating that the Hordeum GPT construct directed the expression of biologically active GPT in a transgenic plant.

### Example 20

### Isolation and Expression of Recombinant Rice GPT Gene Coding Sequence and Analysis of Biological Activity

In this example, the putative coding sequence for rice GPT was isolated and expressed in E. coli. Biologically active recombinant rice GPT was produced, and catalyzed the increased synthesis of 2-oxoglutaramate, as confirmed by HPLC.

### Materials and Methods:

Rice GPT Coding Sequence and Expression in E. coli:

The rice (Oryza sativa) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×106 cells were then assayed for biological activity using the NMR assay, below. Untransformed, wild type E. coli cells were assayed as a control. An additional control used E coli cells transformed with an empty vector.

The DNA sequence of the rice GPT coding sequence used in this example is provided in SEQ ID NO: 10, and the encoded GPT protein amino acid sequence is presented in SEQ ID NO: 11.

HPLC Assay for 2-oxoglutaramate:

HPLC was used to determine 2-oxoglutaramate production in GPT-overexpressing E. coli cells, following a modification of Calderon et al., 1985, J Bacteriol 161(2): 807-809. Briefly, a modified extraction buffer consisting of 25 mM Tris-HCl pH 8.5, 1 mM EDTA, 20 μM Pyridoxal phosphate, 10 mM Cysteine, and ˜1.5% (v/v) Mercaptoethanol was used. Samples (lysate from E. coli cells, 25×106 cells) were added to the extraction buffer at approximately a 1/3 ratio (w/v), incubated for 30 minutes at 37° C., and stopped with 200 μl of 20% TCA. After about 5 minutes, the assay mixture is centrifuged and the supernatant used to quantify 2-oxoglutaramate by HPLC, using an ION-300 7.8 mm ID×30 cm L column, with a mobile phase in 0.01N H₂SO₄, a flow rate of approximately 0.2 ml/min, at 40° C. Injection volume is approximately 20 μl, and retention time between about 38 and 39 minutes. Detection is achieved with 210 nm UV light.

NMR analysis comparison with authentic 2-oxoglutaramate was used to establish that the Arabidopisis full length sequence expresses a GPT with 2-oxoglutaramate synthesis activity. Briefly, authentic 2-oxoglutarmate (structure confirmed with NMR) made by chemical synthesis to validate the HPLC assay, above, by confirming that the product of the assay (molecule synthesized in response to the expressed GPT) and the authentic 2-oxoglutaramate elute at the same retention time. In addition, when mixed together the assay product and the authentic compound elute as a single peak. Furthermore, the validation of the HPLC assay also included monitoring the disappearance of the substrate glutamine and showing that there was a 1:1 molar stoechiometry between glutamine consumed to 2-oxoglutaramte produced. The assay procedure always included two controls, one without the enzyme added and one without the glutamine added. The first shows that the production of the 2-oxoglutaramate was dependent upon having the enzyme present, and the second shows that the production of the 2-oxoglutaramate was dependent upon the substrate glutamine.

### Results:

Expression of the rice GPT coding sequence of SEQ ID NO: 10 resulted in the over-expression of recombinant GPT protein having 2-oxoglutaramate synthesis-catalyzing bioactivity. Specifically, 1.72 nanomoles of 2-oxoglutaramate activity was observed in the E. coli cells overexpressing the recombinant rice GPT, compared to only 0.02 nanomoles of 2-oxoglutaramate activity in control E. coli cells, an 86-fold activity level increase over control.

### Example 21

### Isolation and Expression of Recombinant Soybean GPT Gene Coding Sequence and Analysis of Biological Activity

In this example, the putative coding sequence for soybean GPT was isolated and expressed in E. coli. Biologically active recombinant soybean GPT was produced, and catalyzed the increased synthesis of 2-oxoglutaramate, as confirmed by HPLC.

### Materials and Methods:

Soybean GPT Coding Sequence and Expression in E. coli:

The soybean (Glycine max) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×106 cells were then assayed for biological activity using the HPLC assay, below. Untransformed, wild type E. coli cells were assayed as a control. An additional control used E coli cells transformed with an empty vector.

The DNA sequence of the soybean GPT coding sequence used in this example is provided in SEQ ID NO: 12, and the encoded GPT protein amino acid sequence is presented in SEQ ID NO: 13.

HPLC Assay for 2-oxoglutaramate:

HPLC was used to determine 2-oxoglutaramate production in GPT-overexpressing E. coli cells, as described in Example 20, supra.

### Results:

Expression of the soybean GPT coding sequence of SEQ ID NO: 12 resulted in the over-expression of recombinant GPT protein having 2-oxoglutaramate synthesis-catalyzing bioactivity. Specifically, 31.9 nanomoles of 2-oxoglutaramate activity was observed in the E. coli cells overexpressing the recombinant soybean GPT, compared to only 0.02 nanomoles of 2-oxoglutaramate activity in control E. coli cells, a nearly 1,600-fold activity level increase over control.

### Example 22

### Isolation and Expression of Recombinant Zebra Fish GPT Gene Coding Sequence and Analysis of Biological Activity

In this example, the putative coding sequence for Zebra fish GPT was isolated and expressed in E. coli. Biologically active recombinant Zebra fish GPT was produced, and catalyzed the increased synthesis of 2-oxoglutaramate, as confirmed by HPLC.

### Materials and Methods:

Zebra Fish GPT Coding Sequence and Expression in E. coli:

The Zebra fish (Danio rerio) GPT coding sequence was determined and synthesized, inserted into a PET28 vector, and expressed in E. coli. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×106 cells were then assayed for biological activity using the HPLC assay, below. Untransformed, wild type E. coli cells were assayed as a control. An additional control used E coli cells transformed with an empty vector.

The DNA sequence of the Zebra fish GPT coding sequence used in this example is provided in SEQ ID NO: 16, and the encoded GPT protein amino acid sequence is presented in SEQ ID NO: 17.

HPLC Assay for 2-oxoglutaramate:

HPLC was used to determine 2-oxoglutaramate production in GPT-overexpressing E. coli cells, as described in Example 20, supra.

### Results:

Expression of the Zebra fish GPT coding sequence of SEQ ID NO: 16 resulted in the over-expression of recombinant GPT protein having 2-oxoglutaramate synthesis-catalyzing bioactivity. Specifically, 28.6 nanomoles of 2-oxoglutaramate activity was observed in the E. coli cells overexpressing the recombinant Zebra fish GPT, compared to only 0.02 nanomoles of 2-oxoglutaramate activity in control E. coli cells, a more than 1,400-fold activity level increase over control.

### Example 23

### Generation and Expression of Recombinant Truncated Arabidopsis GPT Gene Coding Sequences and Analysis of Biological Activity

In this example, two different truncations of the Arabidopsis GPT coding sequence were designed and expressed in E. coli, in order to evaluate the activity of GPT proteins in which the putative chloroplast signal peptide is absent or truncated. Recombinant truncated GPT proteins corresponding to the full length Arabidopsis GPT amino acid sequence of SEQ ID NO: 2, truncated to delete either the first 30 amino-terminal amino acid residues, or the first 45 amino-terminal amino acid residues, were successfully expressed and showed biological activity in catalyzing the increased synthesis of 2-oxoglutaramate, as confirmed by HPLC.

### Materials and Methods:

Truncated Arabidopsis GPT Coding Sequences and Expression in E. coli:

The DNA coding sequence of a truncation of the Arabidopsis thaliana GPT coding sequence of SEQ ID NO: 1 was designed, synthesized, inserted into a PET28 vector, and expressed in E. coli. The DNA sequence of the truncated Arabidopsis GPT coding sequence used in this example is provided in SEQ ID NO: 20 (−45 AA construct), and the corresponding truncated GPT protein amino acid sequence is provided in SEQ ID NO: 21. Briefly, E. coli cells were transformed with the expression vector and transformants grown overnight in LB broth diluted and grown to OD 0.4, expression induced with isopropyl-B-D-thiogalactoside (0.4 micromolar), grown for 3 hr and harvested. A total of 25×106 cells were then assayed for biological activity using HPLC as described in Example 20. Untransformed, wild type E. coli cells were assayed as a control. An additional control used E coli cells transformed with an empty vector.

Expression of the truncated −45 Arabidopsis GPT coding sequence of SEQ ID NO: 20 resulted in the over-expression of biologically active recombinant GPT protein (2-oxoglutaramate synthesis-catalyzing bioactivity). Specifically, 16.1 nanomoles of 2-oxoglutaramate activity was observed in the E. coli cells overexpressing the truncated −45 GPT, compared to only 0.02 nanomoles of 2-oxoglutaramate activity in control E. coli cells, a more than 800-fold activity level increase over control. For comparison, the full length Arabidopsis gene coding sequence expressed in the same E. coli assay generated 2.8 nanomoles of 2-oxoglutaramate activity, or roughly less than one-fifth the activity observed from the truncated recombinant GPT protein.

### Example 24

### GPT+GS Transgenic Tobacco Seed Germination Tolerates High Salt Concentrations

In this example, seeds form the double transgene tobacco line XX-3 (Cross 3 in Table 4, see Example 7) were tested in a seed germination assay designed to evaluate tolerance to high salt concentrations.

### Materials and Methods:

Tobacco seeds from the wild type and XX-3 populations were surfaced sterilized (5% bleach solution for 5 minutes followed by a 10% ethanol wash for 3 minutes) and rinsed with sterile distilled water. The surface sterilized seeds were then spread on Murashige and Skoog media (10% agarose) without sucrose and containing either 0 or 200 mM NaCl. The seeds were allowed to germinate in darkness for 2 days followed by 6 days under a 16:8 photoperiod at 24° C. On day eight the rate of germination was determined by measuring the percentage of seeds from the control or transgene plants that had germinated.

### Results:

The results are tabulated in Table XXI below. The rate of germination of the transgenic plant line seeds under zero salt conditions was the same as observed with wild type control plant seeds. In stark contrast, the germination rate of the transgenic plant line seeds under very high salt conditions far exceeded the rate seen in wild type control seeds. Whereas over 81% of the transgenic plant seeds had germinated under the high salt conditions, only about 9% of the wild type control plant seeds had germinated by the same time point. These data indicate that the transgenic seeds are capable of germinating very well under high salt concentrations, an important trait for plant growth in areas of increasingly high water and/or soil salinity.

TABLE XXI
TRANSGENIC TOBACCO PLANTS GERMINATE
AND TOLERATE HIGH SALT

| | Control (0 mM NaCl) | Test (200 mM NaCl)a |
|---|---|---|
| Plant type | % Germination | % Germination |
| Wild type | 92, 87, 94 | 9, 11, 8 |
| Transgene line XX-3 | 92, 91, 94 | 84, 82, 78 |

A10 publications, patents, and patent applications cited in this specification are herein incorporated by reference as if each individual publication or patent application were specifically and individually indicated to be incorporated by reference.

The present invention is not to be limited in scope by the embodiments disclosed herein, which are intended as single illustrations of individual aspects of the invention, and any which are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### Table of Sequences

SEQ ID NO: 1 Arabidopsis glutamine phenylpyruvate transaminase DNA coding sequence:

ATGTACCTGGACATAAATGGTGTGATGATCAAACAGTTTAGCTTCAAAGCCTCTCT
TCTCCCATTCTCTTCTAATTTCCGACAAAGCTCCGCCAAAATCCATCGTCCTATCG
GAGCCACCATGACCACAGTTTCGACTCAGAACGAGTCTACTCAAAAACCCGTCCA
GGTGGCGAAGAGATTAGAGAAGTTCAAGACTACTATTTTCACTCAAATGAGCATAT
TGGCAGTTAAACATGGAGCGATCAATTTAGGCCAAGGCTTTCCCAATTTCGACGG
TCCTGATTTTGTTAAAGAAGCTGCGATCCAAGCTATTAAAGATGGTAAAAACCAGT
ATGCTCGTGGATACGGCATTCCTCAGCTCAACTCTGCTATAGCTGCGCGGTTTCG
TGAAGATACGGGTCTTGTTGTTGATCCTGAGAAAGAAGTTACTGTTACATCTGGTT
GCACAGAAGCCATAGCTGCAGCTATGTTGGGTTTAATAAACCCTGGTGATGAAGT
CATTCTCTTTGCACCGTTTTATGATTCCTATGAAGCAACACTCTCTATGGCTGGTG
CTAAAGTAAAAGGAATCACTTTACGTCCACCGGACTTCTCCATCCCTTTGGAAGAG
CTTAAAGCTGCGGTAACTAACAAGACTCGAGCCATCCTTATGAACACTCCGCACA
ACCCGACCGGGAAGATGTTCACTAGGGAGGAGCTTGAAACCATTGCATCTCTCTG
CATTGAAAACGATGTGCTTGTGTTCTCGGATGAAGTATACGATAAGCTTGCGTTTG
AAATGGATCACATTTCTATAGCTTCTCTTCCCGGTATGTATGAAAGAACTGTGACC
ATGAATTCCCTGGGAAAGACTTTCTCTTTAACCGGATGGAAGATCGGCTGGGCGA
TTGCGCCGCCTCATCTGACTTGGGGAGTTCGACAAGCACACTCTTACCTCACATT
CGCCACATCAACACCAGCACAATGGGCAGCCGTTGCAGCTCTCAAGGCACCAGA
GTCTTACTTCAAAGAGCTGAAAAGAGATTACAATGTGAAAAAGGAGACTCTGGTTA
AGGGTTTGAAGGAAGTCGGATTTACAGTGTTCCCATCGAGCGGGACTTACTTTGT
GGTTGCTGATCACACTCCATTTGGAATGGAGAACGATGTTGCTTTCTGTGAGTATC
TTATTGAAGAAGTTGGGGTCGTTGCGATCCCAACGAGCGTCTTTTATCTGAATCCA
GAAGAAGGGAAGAATTTGGTTAGGTTTGCGTTCTGTAAAGACGAAGAGACGTTGC
GTGGTGCAATTGAGAGGATGAAGCAGAAGCTTAAGAGAAAAGTCTGA

SEQ ID NO: 2 Arabidopsis GPT amino acid sequence

MYLDINGVMIKQFSFKASLLPFSSNFRQSSAKIHRPIGATMTTVSTQNES
TQKPVQVAKRLEKFKTTIFTQMSILAVKHGAINLGQGFPNFDGPDFVKEA
AIQAIKDGKNQYARGYGIPQLNSAIAARFREDTGLVVDPEKEVTVTSGCT
EAIAAAMLGLINPGDEVILFAPFYDSYEATLSMAGAKVKGITLRPPDFSI
PLEELKAAVTNKTRAILMNTPHNPTGKMFTREELETIASLCIENDVLVFS
DEVYDKLAFEMDHISIASLPGMYERTVTMNSLGKTFSLTGWKIGWAIAPP
HLTWGVRQAHSYLTFATSTPAQWAAVAALKAPESYFKELKRDYNVKKETL
VKGLKEVGFTVFPSSGTYFVVADHTPFGMENDVAFCEYLIEEVGVVAIPT
SVFYLNPEEGKNLVRFAFCKDEETLRGAIERMKQKLKRKV

SEQ ID NO: 3 Alfalfa GS1 DNA coding sequence (upper case) with 5′ and 3′ untranslated sequences (indicated in lower case).

atttccgttttcgttttcatttgattcattgaatcaaatcgaatcgaatctttaggattcaatacagat
tccttagattttactaagtttgaaaccaaaaccaaaacATGTCTCTCCTTTCAGATCT
TATCAACCTTGACCTCTCCGAAACCACCGAGAAAATCATCGCCGAAT
ACATATGGATTGGTGGATCTGGTTTGGACTTGAGGAGCAAAGCAAG
GACTCTACCAGGACCAGTTACTGACCCTTCACAGCTTCCCAAGTGGA
ACTATGATGGTTCCAGCACAGGTCAAGCTCCTGGAGAAGATAGTGA
AGTTATTATCTACCCACAAGCCATTTTCAAGGACCCATTTAGAAGGG
GTAACAATATCTTGGTTATGTGTGATGCATACACTCCAGCTGGAGAG
CCCATTCCCACCAACAAGAGACATGCAGCTGCCAAGATTTTCAGCCA
TCCTGATGTTGTTGCTGAAGTACCATGGTATGGTATTGAGCAAGAAT
ACACCTTGTTGCAGAAAGACATCAATTGGCCTCTTGGTTGGCCAGTT
GGTGGTTTTCCTGGACCTCAGGGACCATACTATTGTGGAGCTGGTG
CTGACAAGGCATTTGGCCGTGACATTGTTGACTCACATTACAAAGCC
TGTCTTTATGCCGGCATCAACATCAGTGGAATCAATGGTGAAGTGAT
GCCTGGTCAATGGGAATTCCAAGTTGGTCCCTCAGTTGGTATCTCTG
CTGGTGATGAGATATGGGTTGCTCGTTACATTTTGGAGAGGATCACT
GAGGTTGCTGGTGTGGTGCTTTCCTTTGACCCAAAACCAATTAAGGG
TGATTGGAATGGTGCTGGTGCTCACACAAATTACAGCACCAAGTCTA
TGAGAGAAGATGGTGGCTATGAAGTCATCTTGAAAGCAATTGAGAAG
CTTGGGAAGAAGCACAAGGAGCACATTGCTGCTTATGGAGAAGGCA
ACGAGCGTAGATTGACAGGGCGACATGAGACAGCTGACATTAACAC
CTTCTTATGGGGTGTTGCAAACCGTGGTGCGTCGATTAGAGTTGGAA
GGGACACAGAGAAAGCAGGGAAAGGTTATTTCGAGGATAGGAGGCC
ATCATCTAACATGGATCCATATGTTGTTACTTCCATGATTGCAGACAC
CACCATTCTCTGGAAACCATAAgccaccacacacacatgcattgaagtatttgaaa
gtcattgttgattccgcattagaatttggtcattgttttttctaggatttggatttgtgttattgttatggt
tcacactttgtttgtttgaatttgaggccttgttataggtttcatatttctttctcttgttctaagtaaatg
tcagaataataatgtaat

SEQ ID NO: 4 Alfalfa GS1 amino acid sequence

MSLLSDLINLDLSETTEKIIAEYIWIGGSGLDLRSKARTLPGPVTDPSQL
PKWNYDGSSTGQAPGEDSEVIIYPQAIFKDPFRRGNNILVMCDAYTPAGE
PIPTNKRHAAAKIFSHPDVVAEVPWYGIEQEYTLLQKDINWPLGWPVGGF
PGPQGPYYCGAGADKAFGRDIVDSHYKACLYAGINISGINGEVMPGQWEF
QVGPSVGISAGDEIWVARYILERITEVAGVVLSFDPKPIKGDWNGAGAHT
NYSTKSMREDGGYEVILKAIEKLGKKHKEHIAAYGEGNERRLTGRHETAD
INTFLWGVANRGASIRVGRDTEKAGKGYFEDRRPSSNMDPYVVTSMIADT
TILWKP

SEQ ID NO: 5 Alfalfa GS1 DNA coding sequence (upper case) with 5′ and 3′ untranslated sequences (indicated in lower case) and vector sequences from ClaI to SmaI/SspI and SspI/SmaI to SalI/XhoI (lower case, underlined).

atcgatgaattcgagctcggtacccatttccgttttcgttttcatttgattcattgaatcaaatcgaa
tcgaatctttaggattcaatacagattccttagattttactaagtttgaaaccaaaaccaaaacA
TGTCTCTCCTTTCAGATCTTATCAACCTTGACCTCTCCGAAACCACC
GAGAAAATCATCGCCGAATACATATGGATTGGTGGATCTGGTTTGGA
CTTGAGGAGCAAAGCAAGGACTCTACCAGGACCAGTTACTGACCCT
TCACAGCTTCCCAAGTGGAACTATGATGGTTCCAGCACAGGTCAAG
CTCCTGGAGAAGATAGTGAAGTTATTATCTACCCACAAGCCATTTTC
AAGGACCCATTTAGAAGGGGTAACAATATCTTGGTTATGTGTGATGC
ATACACTCCAGCTGGAGAGCCCATTCCCACCAACAAGAGACATGCA
GCTGCCAAGATTTTCAGCCATCCTGATGTTGTTGCTGAAGTACCATG
GTATGGTATTGAGCAAGAATACACCTTGTTGCAGAAAGACATCAATT
GGCCTCTTGGTTGGCCAGTTGGTGGTTTTCCTGGACCTCAGGGACC
ATACTATTGTGGAGCTGGTGCTGACAAGGCATTTGGCCGTGACATTG
TTGACTCACATTACAAAGCCTGTCTTTATGCCGGCATCAACATCAGT
GGAATCAATGGTGAAGTGATGCCTGGTCAATGGGAATTCCAAGTTG
GTCCCTCAGTTGGTATCTCTGCTGGTGATGAGATATGGGTTGCTCGT
TACATTTTGGAGAGGATCACTGAGGTTGCTGGTGTGGTGCTTTCCTT
TGACCCAAAACCAATTAAGGGTGATTGGAATGGTGCTGGTGCTCACA
CAAATTACAGCACCAAGTCTATGAGAGAAGATGGTGGCTATGAAGTC
ATCTTGAAAGCAATTGAGAAGCTTGGGAAGAAGCACAAGGAGCACA
TTGCTGCTTATGGAGAAGGCAACGAGCGTAGATTGACAGGGCGACA
TGAGACAGCTGACATTAACACCTTCTTATGGGGTGTTGCAAACCGTG
GTGCGTCGATTAGAGTTGGAAGGGACACAGAGAAAGCAGGGAAAGG
TTATTTCGAGGATAGGAGGCCATCATCTAACATGGATCCATATGTTG
TTACTTCCATGATTGCAGACACCACCATTCTCTGGAAACCATAAgcca
ccacacacacatgcattgaagtatttgaaagtcattgttgattccgcattagaatttggtcattgtt
ttttctaggatttggatttgtgttattgttatggttcacactttgtttgtttgaatttgaggccttgttata
gtttcatatttctttctcttgttctaagtaaatgtcagaataataatgtaatggggatcctctagag
tcgag

SEQ ID NO: 6 Arabidopsis GS1 coding sequence
Cambia 1201 vector+rbcS3C+arabidopsis GS1 Bold ATG is the start site,

AAAAAAGAAAAAAAAAACATATCTTGTTTGTCAGTATGGGAAGTTTGAGATAAGGA
CGAGTGAGGGGTTAAAATTCAGTGGCCATTGATTTTGTAATGCCAAGAACCACAA
AATCCAATGGTTACCATTCCTGTAAGATGAGGTTTGCTAACTCTTTTTGTCCGTTA
GATAGGAAGCCTTATCACTATATATACAAGGCGTCCTAATAACCTCTTAGTAACCA
ATTATTTCAGCA [symbol] TCTCTGCTCTCAGATCTCGTTAACCTCAACCTCACCGAT
GCCACCGGGAAAATCATCGCCGAATACATATGGATCGGTGGATCTGGAATGGATA
TCAGAAGCAAAGCCAGGACACTACCAGGACCAGTGACTGATCCATCAAAGCTTCC
CAAGTGGAACTACGACGGATCCAGCACCGGTCAGGCTGCTGGAGAAGACAGTGA
AGTCATTCTATACCCTCAGGCAATATTCAAGGATCCCTTCAGGAAAGGCAACAACA
TCCTGGTGATGTGTGATGCTTACACACCAGCTGGTGATCCTATTCCAACCAACAA
GAGGCACAACGCTGCTAAGATCTTCAGCCACCCCGACGTTGCCAAGGAGGAGCC
TTGGTATGGGATTGAGCAAGAATACACTTTGATGCAAAAGGATGTGAACTGGCCA
ATTGGTTGGCCTGTTGGTGGCTACCCTGGCCCTCAGGGACCTTACTACTGTGGTG
TGGGAGCTGACAAAGCCATTGGTCGTGACATTGTGGATGCTCACTACAAGGCCTG
TCTTTACGCCGGTATTGGTATTTCTGGTATCAATGGAGAAGTCATGCCAGGCCAG
TGGGAGTTCCAAGTCGGCCCTGTTGAGGGTATTAGTTCTGGTGATCAAGTCTGGG
TTGCTCGATACCTTCTCGAGAGGATCACTGAGATCTCTGGTGTAATTGTCAGCTTC
GACCCGAAACCAGTCCCGGGTGACTGGAATGGAGCTGGAGCTCACTGCAACTAC
AGCACTAAGACAATGAGAAACGATGGAGGATTAGAAGTGATCAAGAAAGCGATAG
GGAAGCTTCAGCTGAAACACAAAGAACACATTGCTGCTTACGGTGAAGGAAACGA
GCGTCGTCTCACTGGAAAGCACGAAACCGCAGACATCAACACATTCTCTTGGGGA
GTCGCGAACCGTGGAGCGTCAGTGAGAGTGGGACGTGACACAGAGAAGGAAGG
TAAAGGGTACTTCGAAGACAGAAGGCCAGCTTCTAACATGGATCCTTACGTTGTC
ACCTCCATGATCGCTGAGACGACCATACTCGGTTGA

SEQ ID NO: 7 Arabidopsis GS1 amino acid sequence
Vector sequences at N-terminus in italics

MVDLRNRRTSMSLLSDLVNLNLTDATGKIIAEYIWIGGSGMDIRSKARTL
PGPVTDPSKLPKWNYDGSSTGQAAGEDSEVILYPQAIFKDPFRKGNNILV
MCDAYTPAGDPIPTNKRHNAAKIFSHPDVAKEEPWYGIEQEYTLMQKDVN
WPIGWPVGGYPGPQGPYYCGVGADKAIGRDIVDAHYKACLYAGIGISGIN
GEVMPGQWEFQVGPVEGISSGDQVWVARYLLERITEISGVIVSFDPKPVP
GDWNGAGAHCNYSTKTMRNDGGLEVIKKAIGKLQLKHKEHIAAYGEGNER
RLTGKHETADINTFSWGVANRGASVRVGRDTEKEGKGYFEDRRPASNMDP
YVVTSMIAETTILG

SEQ ID NO: 8 Grape GPT coding DNA sequence
Showing Cambia 1305.1 with (3′ end of) rbcS3C+Vitis vinifera GPT (Grape). Bold ATG is the start site, parentheses are the catI intron and the underlined actagt is the speI cloning site used to splice in the GPT gene.

AAAAAAGAAAAAAAAAACATATCTTGTTTGTCAGTATGGGAAGTTTGAGATAAGGA
CGAGTGAGGGGTTAAAATTCAGTGGCCATTGATTTTGTAATGCCAAGAACCACAA
AATCCAATGGTTACCATTCCTGTAAGATGAGGTTTGCTAACTCTTTTTGTCCGTTA
GATAGGAAGCCTTATCACTATATATACAAGGCGTCCTAATAACCTCTTAGTAACCA
ATTATTTCAGCA [symbol] TAGATCTGAGG(GTAAATTTCTAGTTTTTCTCCTTCATT
TTCTTGGTTAGGACCCTTTTCTCTTTTTATTTTTTTGAGCTTTGATCTTTCTTTAAAC
TGATCTATTTTTTAATTGATTGGTTATGGTGTAAATATTACATAGCTTTAACTGATAA
TCTGATTACTTTATTTCGTGTGTCTATGATGATGATGATAGTTACAG)AACCGACGA [symbol]
ATGCAGCTCTCTCAATGTACCTGGACATTCCCAGAGTTGCTTAAAAGAC
CAGCCTTTTTAAGGAGGAGTATTGATAGTATTTCGAGTAGAAGTAGGTCCAGCTC
CAAGTATCCATCTTTCATGGCGTCCGCATCAACGGTCTCCGCTCCAAATACGGAG
GCTGAGCAGACCCATAACCCCCCTCAACCTCTACAGGTTGCAAAGCGCTTGGAG
AAATTCAAAACAACAATCTTTACTCAAATGAGCATGCTTGCCATCAAACATGGAGC
AATAAACCTTGGCCAAGGGTTTCCCAACTTTGATGGTCCTGAGTTTGTCAAAGAAG
CAGCAATTCAAGCCATTAAGGATGGGAAAAACCAATATGCTCGTGGATATGGAGT
TCCTGATCTCAACTCTGCTGTTGCTGATAGATTCAAGAAGGATACAGGACTCGTG
GTGGACCCCGAGAAGGAAGTTACTGTTACTTCTGGATGTACAGAAGCAATTGCTG
CTACTATGCTAGGCTTGATAAATCCTGGTGATGAGGTGATCCTCTTTGCTCCATTT
TATGATTCCTATGAAGCCACTCTATCCATGGCTGGTGCCCAAATAAAATCCATCAC
TTTACGTCCTCCGGATTTTGCTGTGCCCATGGATGAGCTCAAGTCTGCAATCTCAA
AGAATACCCGTGCAATCCTTATAAACACTCCCCATAACCCCACAGGAAAGATGTTC
ACAAGGGAGGAACTGAATGTGATTGCATCCCTCTGCATTGAGAATGATGTGTTGG
TGTTTACTGATGAAGTTTACGACAAGTTGGCTTTCGAAATGGATCACATTTCCATG
GCTTCTCTTCCTGGGATGTACGAGAGGACCGTGACTATGAATTCCTTAGGGAAAA
CTTTCTCCCTGACTGGATGGAAGATTGGTTGGACAGTAGCTCCCCCACACCTGAC
ATGGGGAGTGAGGCAAGCCCACTCATTCCTCACGTTTGCTACCTGCACCCCAATG
CAATGGGCAGCTGCAACAGCCCTCCGGGCCCCAGACTCTTACTATGAAGAGCTA
AAGAGAGATTACAGTGCAAAGAAGGCAATCCTGGTGGAGGGATTGAAGGCTGTC
GGTTTCAGGGTATACCCATCAAGTGGGACCTATTTTGTGGTGGTGGATCACACCC
CATTTGGGTTGAAAGACGATATTGCGTTTTGTGAGTATCTGATCAAGGAAGTTGG
GGTGGTAGCAATTCCGACAAGCGTTTTCTACTTACACCCAGAAGATGGAAAGAAC
CTTGTGAGGTTTACCTTCTGTAAAGACGAGGGAACTCTGAGAGCTGCAGTTGAAA
GGATGAAGGAGAAACTGAAGCCTAAACAATAGGGGCACGTGA

SEQ ID NO: 9 Grape GPT amino acid sequence

MVDLRNRRTSMQLSQCTWTFPELLKRPAFLRRSIDSISSRSRSSSKYPSF
MASASTVSAPNTEAEQTHNPPQPLQVAKRLEKFKTTIFTQMSMLAIKHGA
INLGQGFPNFDGPEFVKEAAIQAIKDGKNQYARGYGVPDLNSAVADRFKK
DTGLVVDPEKEVTVTSGCTEAIAATMLGLINPGDEVILFAPFYDSYEATL
SMAGAQIKSITLRPPDFAVPMDELKSAISKNTRAILINTPHNPTGKMERT
VTMFTREELNVIASLCIENDVLVFTDEVYDKLAFEMDHISMASLPGMYNS
LGKTFSLTGWKIGWTVAPPHLTWGVRQAHSFLTFATCTPMQWAAATALRA
PDSYYEELKRDYSAKKAILVEGLKAVGFRVYPSSGTYFVVVDHTPFGLKD
DIAFCEYLIKEVGVVAIPTSVFYLHPEDGKNLVRFTFCKDEGTLRAAVER
MKEKLKPKQ

SEQ ID NO: 10 Rice GPT DNA coding sequence
Rice GPT codon optimized for E. coli expression; untranslated sequences shown in lower case

atgtggATGAACCTGGCAGGCTTTCTGGCAACCCCGGCAACCGCAACCGCAACCCG
TCATGAAATGCCGCTGAACCCGAGCAGCAGCGCGAGCTTTCTGCTGAGCAGCCT
GCGTCGTAGCCTGGTGGCGAGCCTGCGTAAAGCGAGCCCGGCAGCAGCAGCAG
CACTGAGCCCGATGGCAAGCGCAAGCACCGTGGCAGCAGAAAACGGTGCAGCA
AAAGCAGCAGCAGAAAAACAGCAGCAGCAGCCGGTGCAGGTGGCGAAACGTCTG
GAAAAATTTAAAACCACCATTTTTACCCAGATGAGCATGCTGGCGATTAAACATGG
CGCGATTAACCTGGGCCAGGGCTTTCC
GAACTTTGATGGCCCGGATTTTGTGAAAGAAGCGGCGATTCAGGCGATTAACGCG
GGCAAAAACCAGTATGCGCGTGGCTATGGCGTGCCGGAACTGAACAGCGCGATT
GCGGAACGTTTTCTGAAAGATAGCGGCCTGCAGGTGGATCCGGAAAAAGAAGTG
ACCGTGACCAGCGGCTGCACCGAAGCGATTGCGGCGACCATTCTGGGCCTGATT
AACCCGGGCGATGAAGTGATTCTGTTTGCGCCGTTTTATGATAGCTATGAAGCGA
CCCTGAGCATGGCGGGCGCGAACGTGAAAGCGATTACCCTGCGTCCGCCGGATT
TTAGCGTGCCGCTGGAAGAACTGAAAGCGGCCGTGAGCAAAAACACCCGTGCGA
TTATGATTAACACCCCGCATAACCCGACCGGCAAAATGTTTACCCGTGAAGAACT
GGAATTTATTGCGACCCTGTGCAAAGAAAACGATGTGCTGCTGTTTGCGGATGAA
GTGTATGATAAACTGGCGTTTGAAGCGGATCATATTAGCATGGCGAGCATTCCGG
GCATGTATGAACGTACCGTGACCATGAACAGCCTGGGCAAAACCTTTAGCCTGAC
CGGCTGGAAAATTGGCTGGGCGATTGCGCCGCCGCATCTGACCTGGGGCGTGC
GTCAGGCACATAGCTTTCTGACCTTTGCAACCTGCACCCCGATGCAGGCAGCCG
CCGCAGCAGCACTGCGTGCACCGGATAGCTATTATGAAGAACTGCGTCGTGATTA
TGGCGCGAAAAAAGCGCTGCTGGTGAACGGCCTGAAAGATGCGGGCTTTATTGT
GTATCCGAGCAGCGGCACCTATTTTGTGATGGTGGATCATACCCCGTTTGGCTTT
GATAACGATATTGAATTTTGCGAATATCTGATTCGTGAAGTGGGCGTGGTGGCGA
TTCCGCCGAGCGTGTTTTATCTGAACCCGGAAGATGGCAAAAACCTGGTGCGTTT
TACCTTTTGCAAAGATGATGAAACCCTGCGTGCGGCGGTGGAACGTATGAAAACC
AAACTGCGTAAAAAAAAGCTTgcggccgcactcgagcaccaccaccaccaccactga

SEQ ID NO: 11 Rice GPT amino acid sequence
Includes amino terminal amino acids MW for cloning and His tag sequences from pet28 vector in italics.

MWMNLAGFLATPATATATRHEMPLNPSSSASFLLSSLRRSLVASLRKASP
AAAAALSPMASASTVAAENGAAKAAAEKQQQQPVQVAKRLEKFKTTIFTQ
MSMLAIKHGAINLGQGFPNFDGPDFVKEAAIQAINAGKNQYARGYGVPEL
NSAIAERFLKDSGLQVDPEKEVTVTSGCTEAIAATILGLINPGDEVILFA
PFYDSYEATLSMAGANVKAITLRPPDFSVPLEELKAAVSKNTRAIMINTP
HNPTGKMFTREELEFIATLCKENDVLLFADEVYDKLAFEADHISMASIPG
MYERTVTMNSLGKTFSLTGWKIGWAIAPPHLTWGVRQAHSFLTFATCTPM
QAAAAAALRAPDSYYEELRRDYGAKKALLVNGLKDAGFIVYPSSGTYFVM
VDHTPFGFDNDIEFCEYLIREVGVVAIPPSVFYLNPEDGKNLVRFTFCKD
DETLRAAVERMKTKLRKKKLAAALEHHHHHH

SEQ ID NO: 12 Soybean GPT DNA coding sequence
TOPO 151D WITH SOYBEAN for E. coli expression
From starting codon. Vector sequences are italicized

ATGCATCATCACCATCACCATGGTAAGCCTATCCCTAACCCTCTCCTCGGTCTCG
ATTCTACGGAAAACCTGTATTTTCAGGGAATTGATCCCTTCACCGCGAAACGTCTG
GAAAAATTTCAGACCACCATTTTTACCCAGATGAGCCTGCTGGCGATTAAACATGG
CGCGATTAACCTGGGCCAGGGCTTTCCGAACTTTGATGGCCCGGAATTTGTGAAA
GAAGCGGCGATTCAGGCGATTCGTGATGGCAAAAACCAGTATGCGCGTGGCTAT
GGCGTGCCGGATCTGAACATTGCGATTGCGGAACGTTTTAAAAAAGATACCGGCC
TGGTGGTGGATCCGGAAAAAGAAATTACCGTGACCAGCGGCTGCACCGAAGCGA
TTGCGGCGACCATGATTGGCCTGATTAACCCGGGCGATGAAGTGATTATGTTTGC
GCCGTTTTATGATAGCTATGAAGCGACCCTGAGCATGGCGGGCGCGAAAGTGAA
AGGCATTACCCTGCGTCCGCCGGATTTTGCGGTGCCGCTGGAAGAACTGAAAAG
CACCATTAGCAAAAACACCCGTGCGATTCTGATTAACACCCCGCATAACCCGACC
GGCAAAATGTTTACCCGTGAAGAACTGAACTGCATTGCGAGCCTGTGCATTGAAA
ACGATGTGCTGGTGTTTACCGATGAAGTGTATGATAAACTGGCGTTTGATATGGA
ACATATTAGCATGGCGAGCCTGCCGGGCATGTTTGAACGTACCGTGACCCTGAAC
AGCCTGGGCAAAACCTTTAGCCTGACCGGCTGGAAAATTGGCTGGGCGATTGCG
CCGCCGCATCTGAGCTGGGGCGTGCGTCAGGCGCATGCGTTTCTGACCTTTGCA
ACCGCACATCCGTTTCAGTGCGCAGCAGCAGCAGCACTGCGTGCACCGGATAGC
TATTATGTGGAACTGAAACGTGATTATATGGCGAAACGTGCGATTCTGATTGAAGG
CCTGAAAGCGGTGGGCTTTAAAGTGTTTCCGAGCAGCGGCACCTATTTTGTGGTG
GTGGATCATACCCCGTTTGGCCTGGAAAACGATGTGGCGTTTTGCGAATATCTGG
TGAAAGAAGTGGGCGTGGTGGCGATTCCGACCAGCGTGTTTTATCTGAACCCGG
AAGAAGGCAAAAACCTGGTGCGTTTTACCTTTTGCAAAGATGAAGAAACCATTCGT
AGCGCGGTGGAACGTATGAAAGCGAAACTGCGTAAAGTCGACTAA

SEQ ID NO: 13 Soybean GPT amino acid sequence
Translated protein product, vector sequences italicized

MHHHHHHGKPIPNPLLGLDSTENLYFQGIDPFTAKRLEKFQTTIFTQMSL
LAIKHGAINLGQGFPNFDGPEFVKEAAIQAIRDGKNQYARGYGVPDLNIA
IAERFKKDTGLVVDPEKEITVTSGCTEAIAATMIGLINPGDEVIMFAPFY
SYEATLSMAGAKVKGITLRPPDFAVPLDEELKSTISKNTRAILINTPHNP
TGKMFTREELNCIASLCIENDVLVFTDEVYDKLAFDMEHISMASLPGMFE
RTVTLNSLGKTFSLTGWKIGWAIAPPHLSWGVRQAHAFLTFATAHPFQCA
AAAALRAPDSYYVELKRDYMAKRAILIEGLKAVGFKVFPSSGTYFVVVDH
TPFGLENDVAFCEYLVKEVGVVAIPTSVFYLNPEEGKNLVRFTFCKDEET
IRSAVERMKAKLRKVD

SEQ ID NO: 14 Barley GPT DNA coding sequence
Coding sequence from start with intron removed

[symbol]

TAGATCTGAGGAACCGACGA [symbol] ATGGCATCCGCCCCCGCCTCCGCC
TCCGCGGCCCTCTCCACCGCCGCCCCCGCCGACAACGGGGCCGCCAAGCCCAC
GGAGCAGCGGCCGGTACAGGTGGCTAAGCGATTGGAGAAGTTCAAAACAACAAT
TTTCACACAGATGAGCATGCTCGCAGTGAAGCATGGAGCAATAAACCTTGGACAG
GGGTTTCCCAATTTTGATGGCCCTGACTTTGTCAAAGATGCTGCTATTGAGGCTAT
CAAAGCTGGAAAGAATCAGTATGCAAGAGGATATGGTGTGCCTGAATTGAACTCA
GCTGTTGCTGAGAGATTTCTCAAGGACAGTGGATTGCACATCGATCCTGATAAGG
AAGTTACTGTTACATCTGGGTGCACAGAAGCAATAGCTGCAACGATATTGGGTCT
GATCAACCCTGGGGATGAAGTCATACTGTTTGCTCCATTCTATGATTCTTATGAGG
CTACACTGTCCATGGCTGGTGCGAATGTCAAAGCCATTACACTCCGCCCTCCGGA
CTTTGCAGTCCCTCTTGAAGAGCTAAAGGCTGCAGTCTCGAAGAATACCAGAGCA
ATAATGATTAATACACCTCACAACCCTACCGGGAAAATGTTCACAAGGGAGGAAC
TTGAGTTCATTGCTGATCTCTGCAAGGAAAATGACGTGTTGCTCTTTGCCGATGAG
GTCTACGACAAGCTGGCGTTTGAGGCGGATCACATATCAATGGCTTCTATTCCTG
GCATGTATGAGAGGACCGTCACTATGAACTCCCTGGGGAAGACGTTCTCCTTGAC
CGGATGGAAGATCGGCTGGGCGATAGCACCACCGCACCTGACATGGGGCGTAA
GGCAGGCACACTCCTTCCTCACATTCGCCACCTCCACGCCGATGCAATCAGCAG
CGGCGGCGGCCCTGAGAGCACCGGACAGCTACTTTGAGGAGCTGAAGAGGGAC
TACGGCGCAAAGAAAGCGCTGCTGGTGGACGGGCTCAAGGCGGCGGGCTTCAT
CGTCTACCCTTCGAGCGGAACCTACTTCATCATGGTCGACCACACCCCGTTCGGG
TTCGACAACGACGTCGAGTTCTGCGAGTACTTGATCCGCGAGGTCGGCGTCGTG
GCCATCCCGCCAAGCGTGTTCTACCTGAACCCGGAGGACGGGAAGAACCTGGTG
AGGTTCACCTTCTGCAAGGACGACGACACGCTAAGGGCGGCGGTGGACAGGATG
AAGGCCAAGCTCAGGAAGAAATGA

SEQ ID NO: 15 Barley GPT amino acid sequence
Translated sequence from start site (intron removed)

MVDLRNRRTSMASAPASASAALSTAAPADNGAAKPTEQRPVQVAKRLEKF
KTTIFTQMSMLAVKHGAINLGQGFPNFDGPDFVKDAAIEAIKAGKNQYAR
GYGVPELNSAVAERFLKDSGLHIDPDKEVTVTSGCTEAIAATILGLINPG
DEVILFAPFYDSYEATLSMAGANVKAITLRPPDFAVPLEELKAAVSKNTR
AIMINTPHNPTGKMFTREELEFIADLCKENDVLLFADEVYDKLAFEADHI
SMASIPGMYERTVTMNSLGKTFSLTGWKIGWAIAPPHLTWGVRQAHSFLT
FATSTPMQSAAAAALRAPDSYFEELKRDYGAKKALLVDGLKAAGFIVYPS
SGTYFIMVDHTPFGFDNDVEFCEYLIREVGVVAIPPSVFYLNPEDGKNLV
RFTFCKDDDTLRAAVDRMKAKLRKK

SEQ ID NO: 16 Zebra fish GPT DNA coding sequence
Danio rerio sequence designed for expression in E coli. Bold, italicized nucleotides added for cloning or from pET28b vector.

[symbol]

GTGGCGAAACGTCTGGAAAAATTTAAAACCACCATTTTTACCCAGATGAG
CATGCTGGCGATTAAACATGGCGCGATTAACCTGGGCCAGGGCTTTCCGAACTTT
GATGGCCCGGATTTTGTGAAAGAAGCGGCGATTCAGGCGATTCGTGATGGCAAC
AACCAGTATGCGCGTGGCTATGGCGTGCCGGATCTGAACATTGCGATTAGCGAA
CGTTATAAAAAAGATACCGGCCTGGCGGTGGATCCGGAAAAAGAAATTACCGTGA
CCAGCGGCTGCACCGAAGCGATTGCGGCGACCGTGCTGGGCCTGATTAACCCG
GGCGATGAAGTGATTGTGTTTGCGCCGTTTTATGATAGCTATGAAGCGACCCTGA
GCATGGCGGGCGCGAAAGTGAAAGGCATTACCCTGCGTCCGCCGGATTTTGCGC
TGCCGATTGAAGAACTGAAAAGCACCATTAGCAAAAACACCCGTGCGATTCTGCT
GAACACCCCGCATAACCCGACCGGCAAAATGTTTACCCCGGAAGAACTGAACAC
CATTGCGAGCCTGTGCATTGAAAACGATGTGCTGGTGTTTAGCGATGAAGTGTAT
GATAAACTGGCGTTTGATATGGAACATATTAGCATTGCGAGCCTGCCGGGCATGT
TTGAACGTACCGTGACCATGAACAGCCTGGGCAAAACCTTTAGCCTGACCGGCTG
GAAAATTGGCTGGGCGATTGCGCCGCCGCATCTGACCTGGGGCGTGCGTCAGG
CGCATGCGTTTCTGACCTTTGCAACCAGCAACCCGATGCAGTGGGCAGCAGCAG
TGGCACTGCGTGCACCGGATAGCTATTATACCGAACTGAAACGTGATTATATGGC
GAAACGTAGCATTCTGGTGGAAGGCCTGAAAGCGGTGGGCTTTAAAGTGTTTCCG
AGCAGCGGCACCTATTTTGTGGTGGTGGATCATACCCCGTTTGGCCATGAAAACG
ATATTGCGTTTTGCGAATATCTGGTGAAAGAAGTGGGCGTGGTGGCGATTCCGAC
CAGCGTGTTTTATCTGAACCCGGAAGAAGGCAAAAACCTGGTGCGTTTTACCTTTT
GCAAAGATGAAGGCACCCTGCGTGCGGCGGTGGATCGTATGAAAGAAAAACTGC
GTAAA

[symbol]

SEQ ID NO: 17 Zebra fish GPR amino acid sequence
Amino acid sequence of Danio rerio cloned and expressed in E. coli (bold, italicized amino acids are added from vector/cloning and His tag on C-terminus)

[symbol]

VAKRLEKFKTTIFTQMSMLAIKHGAINLGQGFPNFDGPDFVKEA
AIQAIRDGNNQYARGYGVPDLNIAISERYKKDTGLAVDPEKEITVTSGCT
EAIAATVLGLINPGDEVIVFAPFYDSYEATLSMAGAKVKGITLRPPDFAL
PIEELKSTISKNTRAILLNTPHNPTGKMFTPEELNTIASLCIENDVLVFS
DEVYDKLAFDMEHISIASLPGMFERTVTMNSLGKTFSLTGWKIGWAIAPP
HLTWGVRQAHAFLTFATSNPMQWAAAVALRAPDSYYTELKRDYMAKRSIL
VEGLKAVGFKVFPSSGTYFVVVDHTPFGHENDIAFCEYLVKEVGVVAIPT
SVFYLNPEEGKNLVRFTFCKDEGTLRAAVDRMKEKLRK

[symbol]

SEQ ID NO: 18 Arabidopsis truncated GPT −30 construct DNA sequence
Arabidopsis GPT coding sequence with 30 amino acids removed from the targeting sequence.

ATGGCCAAAATCCATCGTCCTATCGGAGCCACCATGACCACAGTTTCGACTCAGA
ACGAGTCTACTCAAAAACCCGTCCAGGTGGCGAAGAGATTAGAGAAGTTCAAGAC
TACTATTTTCACTCAAATGAGCATATTGGCAGTTAAACATGGAGCGATCAATTTAG
GCCAAGGCTTTCCCAATTTCGACGGTCCTGATTTTGTTAAAGAAGCTGCGATCCA
AGCTATTAAAGATGGTAAAAACCAGTATGCTCGTGGATACGGCATTCCTCAGCTC
AACTCTGCTATAGCTGCGCGGTTTCGTGAAGATACGGGTCTTGTTGTTGATCCTG
AGAAAGAAGTTACTGTTACATCTGGTTGCACAGAAGCCATAGCTGCAGCTATGTT
GGGTTTAATAAACCCTGGTGATGAAGTCATTCTCTTTGCACCGTTTTATGATTCCT
ATGAAGCAACACTCTCTATGGCTGGTGCTAAAGTAAAAGGAATCACTTTACGTCCA
CCGGACTTCTCCATCCCTTTGGAAGAGCTTAAAGCTGCGGTAACTAACAAGACTC
GAGCCATCCTTATGAACACTCCGCACAACCCGACCGGGAAGATGTTCACTAGGG
AGGAGCTTGAAACCATTGCATCTCTCTGCATTGAAAACGATGTGCTTGTGTTCTCG
GATGAAGTATACGATAAGCTTGCGTTTGAAATGGATCACATTTCTATAGCTTCTCT
TCCCGGTATGTATGAAAGAACTGTGACCATGAATTCCCTGGGAAAGACTTTCTCTT
TAACCGGATGGAAGATCGGCTGGGCGATTGCGCCGCCTCATCTGACTTGGGGAG
TTCGACAAGCACACTCTTACCTCACATTCGCCACATCAACACCAGCACAATGGGC
AGCCGTTGCAGCTCTCAAGGCACCAGAGTCTTACTTCAAAGAGCTGAAAAGAGAT
TACAATGTGAAAAAGGAGACTCTGGTTAAGGGTTTGAAGGAAGTCGGATTTACAG
TGTTCCCATCGAGCGGGACTTACTTTGTGGTTGCTGATCACACTCCATTTGGAAT
GGAGAACGATGTTGCTTTCTGTGAGTATCTTATTGAAGAAGTTGGGGTCGTTGCG
ATCCCAACGAGCGTCTTTTATCTGAATCCAGAAGAAGGGAAGAATTTGGTTAGGTT
TGCGTTCTGTAAAGACGAAGAGACGTTGC
GTGGTGCAATTGAGAGGATGAAGCAGAAGCTTAAGAGAAAAGTCTGA

SEQ ID NO: 19 Arabidopsis truncated GPT −30 construct amino acid sequence

MAKIHRPIGATMTTVSTQNESTQKPVQVAKRLEKFKTTIFTQMSILAVKH
GAINLGQGFPNFDGPDFVKEAAIQAIKDGKNQYARGYGIPQLNSAIAARF
REDTGLVVDPEKEVTVTSGCTEAIAAAMLGLINPGDEVILFAPFYDSYEA
TLSMAGAKVKGITLRPPDFSIPLEELKAAVTNKTRAILMNTPHNPTGKMF
TREELETIASLCIENDVLVFSDEVYDKLAFEMDHISIASLPGMYERTVTM
NSLGKTFSLTGWKIGWAIAPPHLTWGVRQAHSYLTFATSTPAQWAAVAAL
KAPESYFKELKRDYNVKKETLVKGLKEVGFTVFPSSGTYFVVADHTPFGM
ENDVAFCEYLIEEVGVVAIPTSVFYLNPEEGKNLVRFAFCKDEETLRGAI
ERMKQKLKRKV

SEQ ID NO: 20: Arabidopsis truncated GPT −45 construct DNA sequence
Arabidopsis GPT coding sequence with 45 residues in the targeting sequence removed

ATGGCGACTCAGAACGAGTCTACTCAAAAACCCGTCCAGGTGGCGAAGAGATTA
GAGAAGTTCAAGACTACTATTTTCACTCAAATGAGCATATTGGCAGTTAAACATGG
AGCGATCAATTTAGGCCAAGGCTTTCCCAATTTCGACGGTCCTGATTTTGTTAAAG
AAGCTGCGATCCAAGCTATTAAAGATGGTAAAAACCAGTATGCTCGTGGATACGG
CATTCCTCAGCTCAACTCTGCTATAGCTGCGCGGTTTCGTGAAGATACGGGTCTT
GTTGTTGATCCTGAGAAAGAAGTTACTGTTACATCTGGTTGCACAGAAGCCATAG
CTGCAGCTATGTTGGGTTTAATAAACCCTGGTGATGAAGTCATTCTCTTTGCACCG
TTTTATGATTCCTATGAAGCAACACTCTCTATGGCTGGTGCTAAAGTAAAAGGAAT
CACTTTACGTCCACCGGACTTCTCCATCCCTTTGGAAGAGCTTAAAGCTGCGGTA
ACTAACAAGACTCGAGCCATCCTTATGAACACTCCGCACAACCCGACCGGGAAGA
TGTTCACTAGGGAGGAGCTTGAAACCATTGCATCTCTCTGCATTGAAAACGATGT
GCTTGTGTTCTCGGATGAAGTATACGATAAGCTTGCGTTTGAAATGGATCACATTT
CTATAGCTTCTCTTCCCGGTATGTATGAAAGAACTGTGACCATGAATTCCCTGGGA
AAGACTTTCTCTTTAACCGGATGGAAGATCGGCTGGGCGATTGCGCCGCCTCATC
TGACTTGGGGAGTTCGACAAGCACACTCTTACCTCACATTCGCCACATCAACACC
AGCACAATGGGCAGCCGTTGCAGCTCTCAAGGCACCAGAGTCTTACTTCAAAGAG
CTGAAAAGAGATTACAATGTGAAAAAGGAGACTCTGGTTAAGGGTTTGAAGGAAG
TCGGATTTACAGTGTTCCCATCGAGCGGGACTTACTTTGTGGTTGCTGATCACAC
TCCATTTGGAATGGAGAACGATGTTGCTTTCTGTGAGTATCTTATTGAAGAAGTTG
GGGTCGTTGCGATCCCAACGAGCGTCTTTTATCTGAATCCAGAAGAAGGGAAGAA
TTTGGTTAGGTTTGCGTTCTGTAAAGACGAAGAGACGTTGCGTGGTGCAATTGAG
AGGATGAAGCAGAAGCTTAAGAGAAAAGTCTGA

SEQ ID NO: 21: Arabidopsis truncated GPT −45 construct amino acid sequence

MATQNESTQKPVQVAKRLEKFKTTIFTQMSILAVKHGAINLGQGFPNFDG
PDFVKEAAIQAIKDGKNQYARGYGIPQLNSAIAARFREDTGLVVDPEKEV
TVTSGCTEAIAAAMLGLINPGDEVILFAPFYDSYEATLSMAGAKVKGITL
RPPDFSIPLEELKAAVTNKTRAILMNTPHNPTGKMFTREELETIASLCIE
NDVLVFSDEVYDKLAFEMDHISIASLPGMYERTVTMNSLGKTFSLTGWKI
GWAIAPPHLTWGVRQAHSYLTFATSTPAQWAAVAALKAPESYFKELKRDY
NVKKETLVKGLKEVGFTVFPSSGTYFVVADHTPFGMENDVAFCEYLIEEV
GVVAIPTSVFYLNPEEGKNLVRFAFCKDEETLRGAIERMKQKLKRKV

SEQ ID NO: 22: Tomato Rubisco promoter
TOMATO RuBisCo rbcS3C promoter sequence from KpnI to NcoI

GGTACCGTTTGAATCCTCCTTAAAGTTTTTCTCTGGAGAAACTGTAGTAATTTTACT
TTGTTGTGTTCCCTTCATCTTTTGAATTAATGGCATTTGTTTTAATACTAATCTGCTT
CTGAAACTTGTAATGTATGTATATCAGTTTCTTATAATTTATCCAAGTAATATCTTCC
ATTCTCTATGCAATTGCCTGCATAAGCTCGACAAAAGAGTACATCAACCCCTCCTC
CTCTGGACTACTCTAGCTAAACTTGAATTTCCCCTTAAGATTATGAAATTGATATAT
CCTTAACAAACGACTCCTTCTGTTGGAAAATGTAGTACTTGTCTTTCTTCTTTTGGG
TATATATAGTTTATATACACCATACTATGTACAACATCCAAGTAGAGTGAAATGGAT
ACATGTACAAGACTTATTTGATTGATTGATGACTTGAGTTGCCTTAGGAGTAACAA
ATTCTTAGGTCAATAAATCGTTGATTTGAAATTAATCTCTCTGTCTTAGACAGATAG
GAATTATGACTTCCAATGGTCCAGAAAGCAAAGTTCGCACTGAGGGTATACTTGG
AATTGAGACTTGCACAGGTCCAGAAACCAAAGTTCCCATCGAGCTCTAAAATCAC
ATCTTTGGAATGAAATTCAATTAGAGATAAGTTGCTTCATAGCATAGGTAAAATGG
AAGATGTGAAGTAACCTGCAATAATCAGTGAAATGACATTAATACACTAAATACTT
CATATGTAATTATCCTTTCCAGGTTAACAATACTCTATAAAGTAAGAATTATCAGAA
ATGGGCTCATCAAACTTTTGTACTATGTATTTCATATAAGGAAGTATAACTATACAT
AAGTGTATACACAACTTTATTCCTATTTTGTAAAGGTGGAGAGACTGTTTTCGATG
GATCTAAAGCAATATGTCTATAAAATGCATTGATATAATAATTATCTGAGAAAATCC
AGAATTGGCGTTGGATTATTTCAGCCAAATAGAAGTTTGTACCATACTTGTTGATT
CCTTCTAAGTTAAGGTGAAGTATCATTCATAAACAGTTTTCCCCAAAGTACTACTCA
CCAAGTTTCCCTTTGTAGAATTAACAGTTCAAATATATGGCGCAGAAATTACTCTAT
GCCCAAAACCAAACGAGAAAGAAACAAAATACAGGGGTTGCAGACTTTATTTTCG
TGTTAGGGTGTGTTTTTTCATGTAATTAATCAAAAAATATTATGACAAAAACATTTAT
ACATATTTTTACTCAACACTCTGGGTATCAGGGTGGGTTGTGTTCGACAATCAATA
TGGAAAGGAAGTATTTTCCTTATTTTTTTAGTTAATATTTTCAGTTATACCAAACATA
CCTTGTGATATTATTTTTAAAAATGAAAAACTCGTCAGAAAGAAAAAGCAAAAGCAA
CAAAAAAATTGCAAGTATTTTTTAAAAAAGAAAAAAAAAACATATCTTGTTTGTCAG
TATGGGAAGTTTGAGATAAGGACGAGTGAGGGGTTAAAATTCAGTGGCCATTGAT
TTTGTAATGCCAAGAACCACAAAATCCAATGGTTACCATTCCTGTAAGATGAGGTT
TGCTAACTCTTTTTGTCCGTTAGATAGGAAGCCTTATCACTATATATACAAGGCGT
CCTAATAACCTCTTAGTAACCAATTATTTCAGCACC

[symbol]

SEQ ID NO: 23: Bamboo GPT DNA coding sequence

ATGGCCTCCGCGGCCGTCTCCACCGTCGCCACCGCCGCCGACGGCGTCGCGAA
GCCGACGGAGAAGCAGCCGGTACAGGTCGCAAAGCGTTTGGAAAAGTTTAAGAC
AACAATTTTCACACAGATGAGCATGCTTGCCATCAAGCATGGAGCAATAAACCTC
GGCCAGGGCTTTCCGAATTTTGATGGCCCTGACTTTGTGAAAGAAGCTGCTATTC
AAGCTATCAATGCTGGGAAGAATCAGTATGCAAGAGGATATGGTGTGCCTGAACT
GAACTCGGCTGTTGCTGAAAGGTTCCTGAAGGACAGTGGCTTGCAAGTCGATCC
CGAGAAGGAAGTTACTGTCACATCTGGGTGGACGGAAGCGATAGCTGCAACGAT
ATTGGGTCTTATCAACCCTGGCGATGAAGTGATCTTGTTTGCTCCATTCTATGATT
CATACGAGGCTACGCTGTCGATGGCTGGTGCCAATGTAAAAGCCATTACTCTCCG
TCCTCCAGATTTTGCAGTCCCTCTTGAGGAGCTAAAGGCCACAGTCTCTAAGAAC
ACCAGAGCGATAATGATAAACACACCACACAATCCTACTGGGAAAATGTTTTCTAG
GGAAGAACTTGAATTCATTGCTACTCTCTGCAAGAAAAATGATGTGTTGCTTTTTG
CTGATGAGGTCTATGACAAGTTGGCATTTGAGGCAGATCATATATCAATGGCTTCT
ATTCCTGGCATGTATGAGAGGACTGTGACTATGAACTCTCTGGGGAAGACATTCT
CTCTAACAGGATGGAAGATCGGTTGGGCAATAGCACCACCACACCTGACATGGG
GTGTAAGGCAGGCACACTCATTCCTCACATTTGCCACCTGCACACCAATGCAATC
GGCGGCGGCGGCGGCTCTTAGAGCACCAGATAGCTACTATGGGGAGCTGAAGA
GGGATTACGGTGCAAAGAAAGCGATACTAGTCGACGGACTCAAGGCTGCAGGTT
TTATTGTTTACCCTTCAAGTGGAACATACTTTGTCATGGTCGATCACACCCCGTTT
GGTTTCGACAATGATATTGAGTTCTGCGAGTATTTGATCCGCGAAGTCGGTGTTG
TCGCCATACCACCAAGCGTATTTTATCTCAACCCTGAGGATGGGAAGAACTTGGT
GAGGTTCACCTTCTGCAAGGATGATGATACGCTGAGAGCCGCAGTTGAGAGGAT
GAAGACAAAGCTCAGGAAAAAATGA

SEQ ID NO: 24: Bamboo GPT amino acid sequence

MASAAVSTVATAADGVAKPTEKQPVQVAKRLEKFKTTIFTQMSMLAIKHG
AINLGQGFPNFDGPDFVKEAAIQAINAGKNQYARGYGVPELNSAVAERFL
KDSGLQVDPEKEVTVTSGCTEAIAATILGLINPGDEVILFAPFYDSYEAT
LSMAGANVKAITLRPPDFAVPLEELKATVSKNTRAIMINTPHNPTGKMFS
REELEFIATLCKKNDVLLFADEVYDKLAFEADHISMASIPGMYERTVTMN
SLGKTFSLTGWKIGWAIAPPHLTWGVRQAHSFLTFATCTPMQSAAAAALR
APDSYYGELKRDYGAKKAILVDGLKAAGFIVYPSSGTYFVMVDHTPFGFD
NDIEFCEYLIREVGVVAIPPSVFYLNPEDGKNLVRFTFCKDDDTLRAAVE
RMKTKLRKK

SEQ ID NO: 25: 1305.1+rbcS3C promoter+catI intron with rice GPT gene.
Cambia1305.1 with (3′ end of) rbcS3C+rice GPT coding sequence. Underlined ATG is start site, parentheses are the catI intron and the underlined actagt is the speI cloning site used to splice in the rice gene.

AAAAAAGAAAAAAAAAACATATCTTGTTTGTCAGTATGGGAAGTTTGAGATAAGGA
CGAGTGAGGGGTTAAAATTCAGTGGCCATTGATTTTGTAATGCCAAGAACCACAA
AATCCAATGGTTACCATTCCTGTAAGATGAGGTTTGCTAACTCTTTTTGTCCGTTA
GATAGGAAGCCTTATCACTATATATACAAGGCGTCCTAATAACCTCTTAGTAACCA
ATTATTTCAGCA [symbol] TAGATCTGAGG(GTAAATTTCTAGTTTTTCTCCTTCATT
TTCTTGGTTAGGACCCTTTTCTCTTTTTATTTTTTTGAGCTTTGATCTTTCTTTAAAC
TGATCTATTTTTTAATTGATTGGTTATGGTGTAAATATTACATAGCTTTAACTGATAA
TCTGATTACTTTATTTCGTGTGTCTATGATGATGATGATAGTTACAG)AACCGACGA [symbol]
ATGAATCTGGCCGGCTTTCTCGCCACGCCCGCGACCGCGACCGCGACG
CGGCATGAGATGCCGTTAAATCCCTCCTCCTCCGCCTCCTTCCTCCTCTCCTCGC
TCCGCCGCTCGCTCGTCGCGTCGCTCCGGAAGGCCTCGCCGGCGGCGGCCGCG
GCGCTCTCCCCCATGGCCTCCGCGTCCACCGTCGCCGCCGAGAACGGCGCCGC
CAAGGCGGCGGCGGAGAAGCAGCAGCAGCAGCCTGTGCAGGTTGCAAAGCGGT
TGGAAAAGTTTAAGACGACCATTTTCACACAGATGAGTATGCTTGCCATCAAGCAT
GGAGCAATAAACCTTGGCCAGGGTTTTCCGAATTTCGATGGCCCTGACTTTGTAA
AAGAGGCTGCTATTCAAGCTATCAATGCTGGGAAGAATCAGTACGCAAGAGGATA
TGGTGTGCCTGAACTGAACTCAGCTATTGCTGAAAGATTCCTGAAGGACAGCGGA
CTGCAAGTCGATCCGGAGAAGGAAGTTACTGTCACATCTGGATGCACAGAAGCTA
TAGCTGCAACAATTTTAGGTCTAATTAATCCAGGCGATGAAGTGATATTGTTTGCT
CCATTCTATGATTCATATGAGGCTACCCTGTCAATGGCTGGTGCCAACGTAAAAG
CCATTACTCTCCGTCCTCCAGATTTTTCAGTCCCTCTTGAAGAGCTAAAGGCTGCA
GTCTCGAAGAACACCAGAGCTATTATGATAAACACCCCGCACAATCCTACTGGGA
AAATGTTTACAAGGGAAGAACTTGAGTTTATTGCCACTCTCTGCAAGGAAAATGAT
GTGCTGCTTTTTGCTGATGAGGTCTACGACAAGTTAGCTTTTGAGGCAGATCATAT
ATCAATGGCTTCTATTCCTGGCATGTATGAGAGGACCGTGACCATGAACTCTCTT
GGGAAGACATTCTCTCTTACAGGATGGAAGATCGGTTGGGCAATCGCACCGCCA
CACCTGACATGGGGTGTAAGGCAGGCACACTCATTCCTCACGTTTGCGACCTGCA
CACCAATGCAAGCAGCTGCAGCTGCAGCTCTGAGAGCACCAGATAGCTACTATGA
GGAACTGAGGAGGGATTATGGAGCTAAGAAGGCATTGCTAGTCAACGGACTCAA
GGATGCAGGTTTCATTGTCTATCCTTCAAGTGGAACATACTTCGTCATGGTCGACC
ACACCCCATTTGGTTTCGACAATGATATTGAGTTCTGCGAGTATTTGATTCGCGAA
GTCGGTGTTGTCGCCATACCACCTAGTGTATTTTATCTCAACCCTGAGGATGGGA
AGAACTTGGTGAGGTTCACCTTTTGCAAGGATGATGAGACGCTGAGAGCCGCGG
TTGAGAGGATGAAGACAAAGCTCAGGAAAAAATGA

### SEQ ID NO: 26: Hordeum GPT Sequence in Vector

Cambia1305.1 with (3′ end of) rbcS3C+hordeum (IDI4) coding sequence. Underlined ATG is start site, parentheses are the catI intron and the underlined actagt is the speI cloning site used to splice in the hordeum gene.

AAAAAAGAAAAAAAAAACATATCTTGTTTGTCAGTATGGGAAGTTTGAGATAAGGA
CGAGTGAGGGGTTAAAATTCAGTGGCCATTGATTTTGTAATGCCAAGAACCACAA
AATCCAATGGTTACCATTCCTGTAAGATGAGGTTTGCTAACTCTTTTTGTCCGTTA
GATAGGAAGCCTTATCACTATATATACAAGGCGTCCTAATAACCTCTTAGTAACCA
ATTATTTCAGCA [symbol] TAGATCTGAGG(GTAAATTTCTAGTTTTTCTCCTTCATT
TTCTTGGTTAGGACCCTTTTCTCTTTTTATTTTTTTGAGCTTTGATCTTTCTTTAAAC
TGATCTATTTTTTAATTGATTGGTTATGGTGTAAATATTACATAGCTTTAACTGATAA
TCTGATTACTTTATTTCGTGTGTCTATGATGATGATGATAGTTACAG)AACCGACGA [symbol]
ATGGCATCCGCCCCCGCCTCCGCCTCCGCGGCCCTCTCCACCGCCGC
CCCCGCCGACAACGGGGCCGCCAAGCCCACGGAGCAGCGGCCGGTACAGGTG
GCTAAGCGATTGGAGAAGTTCAAAACAACAATTTTCACACAGATGAGCATGCTCG
CAGTGAAGCATGGAGCAATAAACCTTGGACAGGGGTTTCCCAATTTTGATGGCCC
TGACTTTGTCAAAGATGCTGCTATTGAGGCTATCAAAGCTGGAAAGAATCAGTATG
CAAGAGGATATGGTGTGCCTGAATTGAACTCAGCTGTTGCTGAGAGATTTCTCAA
GGACAGTGGATTGCACATCGATCCTGATAAGGAAGTTACTGTTACATCTGGGTGC
ACAGAAGCAATAGCTGCAACGATATTGGGTCTGATCAACCCTGGGGATGAAGTCA
TACTGTTTGCTCCATTCTATGATTCTTATGAGGCTACACTGTCCATGGCTGGTGCG
AATGTCAAAGCCATTACACTCCGCCCTCCGGACTTTGCAGTCCCTCTTGAAGAGC
TAAAGGCTGCAGTCTCGAAGAATACCAGAGCAATAATGATTAATACACCTCACAAC
CCTACCGGGAAAATGTTCACAAGGGAGGAACTTGAGTTCATTGCTGATGTCTGCA
AGGAAAATGACGTGTTGCTCTTTGCCGATGAGGTCTACGACAAGCTGGCGTTTGA
GGCGGATCACATATCAATGGCTTCTATTCCTGGCATGTATGAGAGGACCGTCACT
ATGAACTCCCTGGGGAAGACGTTCTCCTTGACCGGATGGAAGATCGGCTGGGCG
ATAGCACCACCGCACCTGACATGGGGCGTAAGGCAGGCACACTCCTTCCTCACA
TTCGCCACCTCCACGCCGATGCAATCAGCAGCGGCGGCGGCCCTGAGAGCACC
GGACAGCTACTTTGAGGAGCTGAAGAGGGACTACGGCGCAAAGAAAGCGCTGCT
GGTGGACGGGCTCAAGGCGGCGGGCTTCATCGTCTACCCTTCGAGCGGAACCTA
CTTCATCATGGTCGACCACACCCCGTTCGGGTTCGACAACGACGTCGAGTTCTGC
GAGTACTTGATCCGCGAGGTCGGCGTCGTGGCCATCCCGCCAAGCGTGTTCTAC
CTGAACCCGGAGGACGGGAAGAACCTGGTGAGGTTCACCTTCTGCAAGGACGAC
GACACGCTAAGGGCGGCGGTGGACAGGATGAAGGCCAAGCTCAGGAAGAAATG
ATTGAGGGGCG

[symbol]

SEQ ID NO: 27 Cambia 1201+Arabidopsis GPT coding sequence (35S promoter from CaMV in italics)

CATGGAGTCAAAGATTCAAATAGAGGACCTAACAGAACTCGCCGTAAAGACTGGC
GAACAGTTCATACAGAGTCTCTTACGACTCAATGACAAGAAGAAAATCTTCGTCAA
CATGGTGGAGCACGACACACTTGTCTACTCCAAAAATATCAAAGATACAGTCTCA
GAAGACCAAAGGGCAATTGAGACTTTTCAACAAAGGGTAATATCCGGAAACCTCC
TCGGATTCCATTGCCCAGCTATCTGTCACTTTATTGTGAAGATAGTGGAAAAGGAA
GGTGGCTCCTACAAATGCCATCATTGCGATAAAGGAAAGGCCATCGTTGAAGATG
CCTCTGCCGACAGTGGTCCCAAAGATGGACCCCCACCCACGAGGAGCATCGTGG
AAAAAGAAGACGTTCCAACCACGTCTTCAAAGCAAGTGGATTGATGTGATATCTCC
ACTGACGTAAGGGATGACGCACAATCCCACTATCCTTCGCAAGACCCTTCCTCTA
TATAAGGAAGTTCATTTCATTTGGAGAGAACACGGGGGACTCTTGACCATGTACC
TGGACATAAATGGTGTGATGATCAAACAGTTTAGCTTCAAAGCCTCTCTTCTCCCA
TTCTCTTCTAATTTCCGACAAAGCTCCGCCAAAATCCATCGTCCTATCGGAGCCAC
CATGACCACAGTTTCGACTCAGAACGAGTCTACTCAAAAACCCGTCCAGGTGGCG
AAGAGATTAGAGAAGTTCAAGACTACTATTTTCACTCAAATGAGCATATTGGCAGT
TAAACATGGAGCGATCAATTTAGGCCAAGGCTTTCCCAATTTCGACGGTCCTGATT
TTGTTAAAGAAGCTGCGATCCAAGCTATTAAAGATGGTAAAAACCAGTATGCTCGT
GGATACGGCATTCCTCAGCTCAACTCTGCTATAGCTGCGCGGTTTCGTGAAGATA
CGGGTCTTGTTGTTGATCCTGAGAAAGAAGTTACTGTTACATCTGGTTGCACAGAA
GCCATAGCTGCAGCTATGTTGGGTTTAATAAACCCTGGTGATGAAGTCATTCTCTT
TGCACCGTTTTATGATTCCTATGAAGCAACACTCTCTATGGCTGGTGCTAAAGTAA
AAGGAATCACTTTACGTCCACCGGACTTCTCCATCCCTTTGGAAGAGCTTAAAGCT
GCGGTAACTAACAAGACTCGAGCCATCCTTATGAACACTCCGCACAACCCGACCG
GGAAGATGTTCACTAGGGAGGAGCTTGAAACCATTGCATCTCTCTGCATTGAAAA
CGATGTGCTTGTGTTCTCGGATGAAGTATACGATAAGCTTGCGTTTGAAATGGATC
ACATTTCTATAGCTTCTCTTCCCGGTATGTATGAAAGAACTGTGACCATGAATTCC
CTGGGAAAGACTTTCTCTTTAACCGGATGGAAGATCGGCTGGGCGATTGCGCCG
CCTCATCTGACTTGGGGAGTTCGACAAGCACACTCTTACCTCACATTCGCCACAT
CAACACCAGCACAATGGGCAGCCGTTGCAGCTCTCAAGGCACCAGAGTCTTACTT
CAAAGAGCTGAAAAGAGATTACAATGTGAAAAAGGAGACTCTGGTTAAGGGTTTG
AAGGAAGTCGGATTTACAGTGTTCCCATCGAGCGGGACTTACTTTGTGGTTGCTG
ATCACACTCCATTTGGAATGGAGAACGATGTTGCTTTCTGTGAGTATCTTATTGAA
GAAGTTGGGGTCGTTGCGATCCCAACGAGCGTCTTTTATCTGAATCCAGAAGAAG
GGAAGAATTTGGTTAGGTTTGCGTTCTGTAAAGACGAAGAGACGTTGCGTGGTGC
AATTGAGAGGATGAAGCAGAAGCTTAAGAGAAAAGTCTGA

SEQ, ID NO: 28 Cambia p1305.1 with (3′ end of) rbcS3C+Arabidopsis GPT coding sequence. Underlined ATG is start site, parentheses are the catI intron and the underlined actagt is the speI cloning site used to splice in the Arabidopsis gene.

AAAAAAGAAAAAAAAAACATATCTTGTTTGTCAGTATGGGAAGTTTGAGATAAGGA
CGAGTGAGGGGTTAAAATTCAGTGGCCATTGATTTTGTAATGCCAAGAACCACAA
AATCCAATGGTTACCATTCCTGTAAGATGAGGTTTGCTAACTCTTTTTGTCCGTTA
GATAGGAAGCCTTATCACTATATATACAAGGCGTCCTAATAACCTCTTAGTAACCA
ATTATTTCAGCA [symbol] TAGATCTGAGG(GTAAATTTCTAGTTTTTCTCCTTCATT
TTCTTGGTTAGGACCCTTTTCTCTTTTTATTTTTTTGAGCTTTGATCTTTCTTTAAAC
TGATCTATTTTTTAATTGATTGGTTATGGTGTAAATATTACATAGCTTTAACTGATAA
TCTGATTACTTTATTTCGTGTGTCTATGATGATGATGATAGTTAGAG)AACCGACGA [symbol]
ATGTACCTGGACATAAATGGTGTGATGATCAAACAGTTTAGCTTCAAAGC
CTCTCTTCTCCCATTCTCTTCTAATTTCCGACAAAGCTCCGCCAAAATCCATCGTC
CTATCGGAGCCACCATGACCACAGTTTCGACTCAGAACGAGTCTACTCAAAAACC
CGTCCAGGTGGCGAAGAGATTAGAGAAGTTCAAGACTACTATTTTCACTCAAATG
AGCATATTGGCAGTTAAACATGGAGCGATCAATTTAGGCCAAGGCTTTCCCAATTT
CGACGGTCCTGATTTTGTTAAAGAAGCTGCGATCCAAGCTATTAAAGATGGTAAAA
ACCAGTATGCTCGTGGATACGGCATTCCTCAGCTCAACTCTGCTATAGCTGCGCG
GTTTCGTGAAGATACGGGTCTTGTTGTTGATCCTGAGAAAGAAGTTACTGTTACAT
CTGGTTGCACAGAAGCCATAGCTGCAGCTATGTTGGGTTTAATAAACCCTGGTGA
TGAAGTCATTCTCTTTGCACCGTTTTATGATTCCTATGAAGCAACACTCTCTATGG
CTGGTGCTAAAGTAAAAGGAATCACTTTACGTCCACCGGACTTCTCCATCCCTTTG
GAAGAGCTTAAAGCTGCGGTAACTAACAAGACTCGAGCCATCCTTATGAACACTC
CGCACAACCCGACCGGGAAGATGTTCACTAGGGAGGAGCTTGAAACCATTGCAT
CTCTCTGCATTGAAAACGATGTGCTTGTGTTCTCGGATGAAGTATACGATAAGCTT
GCGTTTGAAATGGATCACATTTCTATAGCTTCTCTTCCCGGTATGTATGAAAGAAC
TGTGACCATGAATTCCCTGGGAAAGACTTTCTCTTTAACCGGATGGAAGATCGGC
TGGGCGATTGCGCCGCCTCATCTGACTTGGGGAGTTCGACAAGCACACTCTTAC
CTCACATTCGCCACATCAACACCAGCACAATGGGCAGCCGTTGCAGCTCTCAAGG
CACCAGAGTCTTACTTCAAAGAGCTGAAAAGAGATTACAATGTGAAAAAGGAGAC
TCTGGTTAAGGGTTTGAAGGAAGTCGGATTTACAGTGTTCCCATCGAGCGGGACT
TACTTTGTGGTTGCTGATCACACTCCATTTGGAATGGAGAACGATGTTGCTTTCTG
TGAGTATCTTATTGAAGAAGTTGGGGTCGTTGCGATCCCAACGAGCGTCTTTTATC
TGAATCCAGAAGAAGGGAAGAATTTGGTTAGGTTTGCGTTCTGTAAAGACGAAGA
GACGTTGCGTGGTGCAATTGAGAGGATGAAGCAGAAGCTTAAGAGAAAAGTCTGA

SEQ ID NO: 29 Arabidopsis GPT coding sequence (mature protein, no targeting sequence)

GTGGCGAAGAGATTAGAGAAGTTCAAGACTACTATTTTCACTCAAATGAGCATATT
GGCAGTTAAACATGGAGCGATCAATTTAGGCCAAGGCTTTCCCAATTTCGACGGT
CCTGATTTTGTTAAAGAAGCTGCGATCCAAGCTATTAAAGATGGTAAAAACCAGTA
TGCTCGTGGATACGGCATTCCTCAGCTCAACTCTGCTATAGCTGCGCGGTTTCGT
GAAGATACGGGTCTTGTTGTTGATCCTGAGAAAGAAGTTACTGTTACATCTGGTTG
CACAGAAGCCATAGCTGCAGCTATGTTGGGTTTAATAAACCCTGGTGATGAAGTC
ATTCTCTTTGCACCGTTTTATGATTCCTATGAAGCAACACTCTCTATGGCTGGTGC
TAAAGTAAAAGGAATCACTTTACGTCCACCGGACTTCTCCATCCCTTTGGAAGAGC
TTAAAGCTGCGGTAACTAACAAGACTCGAGCCATCCTTATGAACACTCCGCACAA
CCCGACCGGGAAGATGTTCACTAGGGAGGAGCTTGAAACCATTGCATCTCTCTGC
ATTGAAAACGATGTGCTTGTGTTCTCGGATGAAGTATACGATAAGCTTGCGTTTGA
AATGGATCACATTTCTATAGCTTCTCTTCCCGGTATGTATGAAAGAACTGTGACCA
TGAATTCCCTGGGAAAGACTTTCTCTTTAACCGGATGGAAGATCGGCTGGGCGAT
TGCGCCGCCTCATCTGACTTGGGGAGTTCGACAAGCACACTCTTACCTCACATTC
GCCACATCAACACCAGCACAATGGGCAGCCGTTGCAGCTCTCAAGGCACCAGAG
TCTTACTTCAAAGAGCTGAAAAGAGATTACAATGTGAAAAAGGAGACTCTGGTTAA
GGGTTTGAAGGAAGTCGGATTTACAGTGTTCCCATCGAGCGGGACTTACTTTGTG
GTTGCTGATCACACTCCATTTGGAATGGAGAACGATGTTGCTTTCTGTGAGTATCT
TATTGAAGAAGTTGGGGTCGTTGCGATCCCAACGAGCGTCTTTTATCTGAATCCA
GAAGAAGGGAAGAATTTGGTTAGGTTTGCGTTCTGTAAAGACGAAGAGACGTTGC
GTGGTGCAATTGAGAGGATGAAGCAGAAGCTTAAGAGAAAAGTCTGA

SEQ ID NO: 30 Arabidopsis GPT amino acid sequence (mature protein, no targeting sequence)

VAKRLEKFKTTIFTQMSILAVKHGAINLGQGFPNFDGPDFVKEAAIQAIK
DGKNQYARGYGIPQLNSAIAARFREDTGLVVDPEKEVTVTSGCTEAIAAA
MLGLINPGDEVILFAPFYDSYEATLSMAGAKVKGITLRPPDFSIPLEELK
AAVTNKTRAILMNTPHNPTGKMFTREELETIASLCIENDVLVFSDEVYDK
LAFEMDHISIASLPGMYERTVTMNSLGKTFSLTGWKIGWAIAPPHLTWGV
RQAHSYLTFATSTPAQWAAVAALKAPESYFKELKRDYNVKKETLVKGLKE
VGFTVFPSSGTYFVVADHTPFGMENDVAFCEYLIEEVGVVAIPTSVFYLN
PEEGKNLVRFAFCKDEETLRGAIERMKQKLKRKV

SEQ ID NO: 31 Grape GPT amino acid sequence (mature protein, no targeting sequence)

VAKRLEKFKTTIFTQMSMLAIKHGAINLGQGFPNFDGPEFVKEAAIQAIK
DGKNQYARGYGVPDLNSAVADRFKKDTGLVVDPEKEVTVTSGCTEAIAAT
MLGLINPGDEVILFAPFYDSYEATLSMAGAQIKSITLRPPDFAVPMDELK
SAISKNTRAILINTPHNPTGKMFTREELNVIASLCIENDVLVFTDEVYDK
LAFEMDHISMASLPGMYERTVTMNSLGKTFSLTGWKIGWTVAPPHLTWGV
RQAHSFLTFATCTPMQWAAATALRAPDSYYEELKRDYSAKKAILVEGLKA
VGFRVYPSSGTYFVVVDHTPFGLKDDIAFCEYLIKEVGVVAIPTSVFYLH
PEDGKNLVRFTFCKDEGTLRAAVERMKEKLKPKQ

SEQ ID NO: 32 Rice GPT amino acid sequence (mature protein, no targeting sequence)

VAKRLEKFKTTIFTQMSMLAIKHGAINLGQGFPNFDGPDFVKEAAIQAIN
AGKNQYARGYGVPELNSAIAERFLKDSGLQVDPEKEVTVTSGCTEAIAAT
ILGLINPGDEVILFAPFYDSYEATLSMAGANVKAITLRPPDFSVPLEELK
AAVSKNTRAIMINTPHNPTGKMFTREELEFIATLCKENDVLLFADEVYDK
LAFEADHISMASIPGMYERTVTMNSLGKTFSLTGWKIGWAIAPPHLTWGV
RQAHSFLTFATCTPMQAAAAAALRAPDSYYEELRRDYGAKKALLVNGLKD
AGFIVYPSSGTYFVMVDHTPFGFDNDIEFCEYLIREVGVVAIPPSVFYLN
PEDGKNLVRFTFCKDDETLRAAVERMKTKLRKK

SEQ ID NO: 33 Soybean GPT amino acid sequence (−1 mature protein, no targeting sequence)

AKRLEKFQTTIFTQMSLLAIKHGAINLGQGFPNFDGPEFVKEAAIQAIRD
GKNQYARGYGVPDLNIAIAERFKKDTGLVVDPEKEITVTSGCTEAIAATM
IGLINPGDEVIMFAPFYDSYEATLSMAGAKVKGITLRPPDFAVPLEELKS
TISKNTRAILINTPHNPTGKMFTREELNCIASLCIENDVLVFTDEVYDKL
AFDMEHISMASLPGMFERTVTLNSLGKTFSLTGWKIGWAIAPPHLSWGVR
QAHAFLTFATAHPFQCAAAAALRAPDSYYVELKRDYMAKRAILIEGLKAV
GFKVFPSSGTYFVVVDHTPFGLENDVAFCEYLVKEVGVVAIPTSVFYLNP
EEGKNLVRFTFCKDEETIRSAVERMKAKLRKVD

SEQ ID NO: 34 Barley GPT amino acid sequence (mature protein, no targeting sequence)

VAKRLEKFKTTIFTQMSMLAVKHGAINLGQGFPNFDGPDFVKDAAIEAIK
AGKNQYARGYGVPELNSAVAERFLKDSGLHIDPDKEVTVTSGCTEAIAAT
ILGLINPGDEVILFAPFYDSYEATLSMAGANVKAITLRPPDFAVPLEELK
AAVSKNTRAIMINTPHNPTGKMFTREELEFIADLCKENDVLLFADEVYDK
LAFEADHISMASIPGMYERTVTMNSLGKTFSLTGWKIGWAIAPPHLTWGV
RQAHSFLTFATSTPMQSAAAAALRAPDSYFEELKRDYGAKKALLVDGLKA
AGFIVYPSSGTYFIMVDHTPFGFDNDVEFCEYLIREVGVVAIPPSVFYLN
PEDGKNLVRFTFCKDDDTLRAAVDRMKAKLRKK

SEQ ID NO: 35 Zebra fish GPT amino acid sequence (mature protein, no targeting sequence)

VAKRLEKFKTTIFTQMSMLAIKHGAINLGQGFPNFDGPDFVKEAAIQAIR
DGNNQYARGYGVPDLNIAISERYKKDTGLAVDPEKEITVTSGCTEAIAAT
VLGLINPGDEVIVFAPFYDSYEATLSMAGAKVKGITLRPPDFALPIEELK
STISKNTRAILLNTPHNPTGKMFTPEELNTIASLCIENDVLVFSDEVYDK
LAFDMEHISIASLPGMFERTVTMNSLGKTFSLTGWKIGWAIAPPHLTWGV
RQAHAFLTFATSNPMQWAAAVALRAPDSYYTELKRDYMAKRSILVEGLKA
VGFKVFPSSGTYFVVVDHTPFGHENDIAFCEYLVKEVGVVAIPTSVFYLN
PEEGKNLVRFTFCKDEGTLRAAVDRMKEKLRK

SEQ ID NO: 36 Bamboo GPT amino acid sequence (mature protein, no targeting sequence)

VAKRLEKFKTTIFTQMSMLAIKHGAINLGQGFPNFDGPDFVKEAAIQAIN
AGKNQYARGYGVPELNSAVAERFLKDSGLQVDPEKEVTVTSGCTEAIAAT
ILGLINPGDEVILFAPFYDSYEATLSMAGANVKAITLRPPDFAVPLEELK
ATVSKNTRAIMINTPHNPTGKMFSREELEFIATLCKKNDVLLFADEVYDK
LAFEADHISMASIPGMYERTVTMNSLGKTFSLTGWKIGWAIAPPHLTWGV
RQAHSFLTFATCTPMQSAAAAALRAPDSYYGELKRDYGAKKAILVDGLKA
AGFIVYPSSGTYFVMVDHTPFGFDNDIEFCEYLIREVGVVAIPPSVFYLN
PEDGKNLVRFTFCKDDDTLRAAVERMKTKLRKK

1. A transgenic plant comprising a GPT transgene and a GS transgene, wherein each of said GPT transgene and said GS transgene is operably linked to a plant promoter.

2. The transgenic plant of claim 1, wherein the GS transgene is a GS1 transgene.

3. The transgenic plant of claim 1, wherein the GPT transgene encodes a polypeptide having an amino acid sequence selected from the group consisting of (a) SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO:31, SEQ ID NO: 32, SEQ ID NO: 33, SEQ ID NO: 34, SEQ ID NO: 35 and SEQ ID NO: 36, and (b) an amino acid sequence that is at least 75% identical to any one of SEQ ID NO: 2; SEQ ID NO: 9; SEQ ID NO: 15, SEQ ID NO: 19, SEQ ID NO: 21, SEQ ID NO 24, SEQ ID NO: 30, SEQ ID NO: 31, SEQ ID NO: 32, SEQ ID NO: 33, SEQ ID NO: 34, SEQ ID NO: 35 and SEQ ID NO: 36 and has GPT activity.

4. The transgenic plant of claim 3, wherein the GS transgene encodes a polypeptide having an amino acid sequence selected form the group consisting of (a) SEQ ID NO: 4 and SEQ ID NO: 7 from residue 11, and (b) an amino acid sequence that is at least 75% identical to SEQ ID NO: 4 or SEQ ID NO: 7.

5. The transgenic plant according to claim 1, wherein the GPT and GS transgenes are incorporated into the genome of the plant.

6. The transgenic plant of claim 5, further defined as a monocotyledonous plant.

7. The transgenic plant of claim 5, further defined as a dicotyledonous plant.

8. A progeny of any generation of the transgenic plant of claim 5, wherein said progeny comprises said GPT transgene and said GS transgene.

9. A seed of any generation of the transgenic plant of claim 5, wherein said seed comprises said GPT transgene and said GS transgene.

10. The transgenic plant of claim 5 which displays an enhanced growth rate, increased biomass yield, increased seed yield, increased flower or flower bud yield, increased fruit or pod yield, larger leaves, increased GPT activity, increased GS activity, increased 2-oxoglutaramate levels, increased nitrogen use efficiency, and/or increased tolerance to salt or saline conditions when compared to an analogous wild-type or untransformed plant.

11.-20. (canceled)

21. A method for producing a plant having enhanced growth properties relative to an analogous wild type or untransformed plant, comprising:
(a) introducing a GPT transgene into the plant;
(b) introducing a GS transgene into the plant or a progeny of the plant;
(c) expressing the GPT transgene and the GS transgene in the plant or the progeny of the plant; and,
(d) selecting a plant having an increased growth characteristic relative to a plant of the same species that does not comprise a GPT transgene or a GS transgene.

22. A method for producing a plant having enhanced growth properties relative to an analogous wild type or untransformed plant, comprising:
(a) introducing a GS transgene into the plant;
(b) introducing a GPT transgene into the plant or a progeny of the plant;
(c) expressing the GS transgene and the GPT transgene in the plant or the progeny of the plant; and,
(d) selecting a plant having an increased growth characteristic relative to a plant of the same species that does not comprise a GS transgene or a GPT transgene.

23. The method according to claim 21, wherein the increased growth characteristic is selected from the group consisting of increased biomass, earlier flowering, earlier budding, increased plant height, increased flowering, increased budding, larger leaves, increased fruit or pod yield and increased seed yield.

24. A method of producing a plant having increased nitrogen use efficiency relative to an analogous wild type or untransformed plant, comprising:
(a) introducing a GPT transgene into the plant;
(b) introducing a GS transgene into the plant or a progeny of the plant;
(c) expressing the GPT transgene and the GS transgene in the plant or the progeny of the plant; and,
(d) selecting a plant having an increased nitrogen use efficiency relative to a plant of the same species that does not comprise a GPT transgene or a GS transgene.

25. A method of producing a plant having increased nitrogen use efficiency relative to an analogous wild type or untransformed plant, comprising:
(a) introducing a GS transgene into the plant;
(b) introducing a GPT transgene into the plant or a progeny of the plant;
(c) expressing the GS transgene and the GPT transgene in the plant or the progeny of the plant; and,
(d) selecting a plant having an increased nitrogen use efficiency relative to a plant of the same species that does not comprise a GS transgene or a GPT transgene.

26. A method of producing a plant seed having increased tolerance to germination or growth in salt or saline conditions relative to an analogous wild type or untransformed plant, comprising:
(a) introducing a GPT transgene into the plant;
(b) introducing a GS transgene into the plant or a progeny of the plant;
(c) expressing the GPT transgene and the GS transgene in the plant or the progeny of the plant;
(d) selecting a plant having an increased growth characteristic relative to a plant of the same species that does not comprise a GPT transgene or a GS transgene; and,
(e) harvesting seeds from said plant and selecting for seeds that demonstrate increased germination in high salt conditions.

27. A method of producing a plant seed having increased tolerance to germination in salt or saline conditions relative to an analogous wild type or untransformed plant, comprising:
(a) introducing a GS transgene into the plant;
(b) introducing a GPT transgene into the plant or a progeny of the plant;
(c) expressing the GS transgene and the GPT transgene in the plant or the progeny of the plant;
(d) selecting a plant having an increased growth characteristic relative to a plant of the same species that does not comprise a GS transgene or a GPT transgene; and,
(e) harvesting seeds from said plant and selecting a seed that demonstrates increased germination in high salt conditions.

28. The method according to claim 26, further comprising propagating a plant from the seed so selected and harvesting a seed therefrom.

29. The method according to claim 22, wherein the increased growth characteristic is selected from the group consisting of increased biomass, earlier flowering, earlier budding, increased plant height, increased flowering, increased budding, larger leaves, increased fruit or pod yield and increased seed yield.

30. The method according to claim 27, further comprising propagating a plant from the seed so selected and harvesting a seed therefrom.


SEQUENCE LISTING (38 sequences)

SEQ ID NO: 1 - DNA, 1323 bp, Arabidopsis thaliana
atgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 2 - protein, 440 aa, Arabidopsis thaliana
MYLDINGVMIKQFSFKASLLPFSSNFRQSSAKIHRPIGATMTTVSTQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 3 - DNA, 1374 bp, Medicago sativa
atttccgttttcgttttcatttgattcattgaatcaaatcgaatcgaatctttaggattcaatacagattccttagattttactaagtttgaaaccaaaaccaaaacatgtctctcctttcagatcttatcaaccttgacctctccgaaaccaccgagaaaatcatcgccgaatacatatggattggtggatctggtttggacttgaggagcaaagcaaggactctaccaggaccagttactgacccttcacagcttcccaagtggaactatgatggttccagcacaggtcaagctcctggagaagatagtgaagttattatctacccacaagccattttcaaggacccatttagaaggggtaacaatatcttggttatgtgtgatgcatacactccagctggagagcccattcccaccaacaagagacatgcagctgccaagattttcagccatcctgatgttgttgctgaagtaccatggtatggtattgagcaagaatacaccttgttgcagaaagacatcaattggcctcttggttggccagttggtggttttcctggacctcagggaccatactattgtggagctggtgctgacaaggcatttggccgtgacattgttgactcacattacaaagcctgtctttatgccggcatcaacatcagtggaatcaatggtgaagtgatgcctggtcaatgggaattccaagttggtccctcagttggtatctctgctggtgatgagatatgggttgctcgttacattttggagaggatcactgaggttgctggtgtggtgctttcctttgacccaaaaccaattaagggtgattggaatggtgctggtgctcacacaaattacagcaccaagtctatgagagaagatggtggctatgaagtcatcttgaaagcaattgagaagcttgggaagaagcacaaggagcacattgctgcttatggagaaggcaacgagcgtagattgacagggcgacatgagacagctgacattaacaccttcttatggggtgttgcaaaccgtggtgcgtcgattagagttggaagggacacagagaaagcagggaaaggttatttcgaggataggaggccatcatctaacatggatccatatgttgttacttccatgattgcagacaccaccattctctggaaaccataagccaccacacacacatgcattgaagtatttgaaagtcattgttgattccgcattagaatttggtcattgttttttctaggatttggatttgtgttattgttatggttcacactttgtttgtttgaatttgaggccttgttataggtttcatatttctttctcttgttctaagtaaatgtcagaataataatgtaat

SEQ ID NO: 4 - protein, 356 aa, Medicago sativa
MSLLSDLINLDLSETTEKIIAEYIWIGGSGLDLRSKARTLPGPVTDPSQLPKWNYDGSSTGQAPGEDSEVIIYPQAIFKDPFRRGNNILVMCDAYTPAGEPIPTNKRHAAAKIFSHPDVVAEVPWYGIEQEYTLLQKDINWPLGWPVGGFPGPQGPYYCGAGADKAFGRDIVDSHYKACLYAGINISGINGEVMPGQWEFQVGPSVGISAGDEIWVARYILERITEVAGVVLSFDPKPIKGDWNGAGAHTNYSTKSMREDGGYEVILKAIEKLGKKHKEHIAAYGEGNERRLTGRHETADINTFLWGVANRGASIRVGRDTEKAGKGYFEDRRPSSNMDPYVVTSMIADTTILWKP

SEQ ID NO: 5 - DNA, 1419 bp, Medicago sativa
atcgatgaattcgagctcggtacccatttccgttttcgttttcatttgattcattgaatcaaatcgaatcgaatctttaggattcaatacagattccttagattttactaagtttgaaaccaaaaccaaaacatgtctctcctttcagatcttatcaaccttgacctctccgaaaccaccgagaaaatcatcgccgaatacatatggattggtggatctggtttggacttgaggagcaaagcaaggactctaccaggaccagttactgacccttcacagcttcccaagtggaactatgatggttccagcacaggtcaagctcctggagaagatagtgaagttattatctacccacaagccattttcaaggacccatttagaaggggtaacaatatcttggttatgtgtgatgcatacactccagctggagagcccattcccaccaacaagagacatgcagctgccaagattttcagccatcctgatgttgttgctgaagtaccatggtatggtattgagcaagaatacaccttgttgcagaaagacatcaattggcctcttggttggccagttggtggttttcctggacctcagggaccatactattgtggagctggtgctgacaaggcatttggccgtgacattgttgactcacattacaaagcctgtctttatgccggcatcaacatcagtggaatcaatggtgaagtgatgcctggtcaatgggaattccaagttggtccctcagttggtatctctgctggtgatgagatatgggttgctcgttacattttggagaggatcactgaggttgctggtgtggtgctttcctttgacccaaaaccaattaagggtgattggaatggtgctggtgctcacacaaattacagcaccaagtctatgagagaagatggtggctatgaagtcatcttgaaagcaattgagaagcttgggaagaagcacaaggagcacattgctgcttatggagaaggcaacgagcgtagattgacagggcgacatgagacagctgacattaacaccttcttatggggtgttgcaaaccgtggtgcgtcgattagagttggaagggacacagagaaagcagggaaaggttatttcgaggataggaggccatcatctaacatggatccatatgttgttacttccatgattgcagacaccaccattctctggaaaccataagccaccacacacacatgcattgaagtatttgaaagtcattgttgattccgcattagaatttggtcattgttttttctaggatttggatttgtgttattgttatggttcacactttgtttgtttgaatttgaggccttgttataggtttcatatttctttctcttgttctaagtaaatgtcagaataataatgtaatggggatcctctagagtcgag

SEQ ID NO: 6 - DNA, 1302 bp, Artificial Sequence
  Synthetic plasmid vector sequence
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatgtctctgctctcagatctcgttaacctcaacctcaccgatgccaccgggaaaatcatcgccgaatacatatggatcggtggatctggaatggatatcagaagcaaagccaggacactaccaggaccagtgactgatccatcaaagcttcccaagtggaactacgacggatccagcaccggtcaggctgctggagaagacagtgaagtcattctataccctcaggcaatattcaaggatcccttcaggaaaggcaacaacatcctggtgatgtgtgatgcttacacaccagctggtgatcctattccaaccaacaagaggcacaacgctgctaagatcttcagccaccccgacgttgccaaggaggagccttggtatgggattgagcaagaatacactttgatgcaaaaggatgtgaactggccaattggttggcctgttggtggctaccctggccctcagggaccttactactgtggtgtgggagctgacaaagccattggtcgtgacattgtggatgctcactacaaggcctgtctttacgccggtattggtatttctggtatcaatggagaagtcatgccaggccagtgggagttccaagtcggccctgttgagggtattagttctggtgatcaagtctgggttgctcgataccttctcgagaggatcactgagatctctggtgtaattgtcagcttcgacccgaaaccagtcccgggtgactggaatggagctggagctcactgcaactacagcactaagacaatgagaaacgatggaggattagaagtgatcaagaaagcgatagggaagcttcagctgaaacacaaagaacacattgctgcttacggtgaaggaaacgagcgtcgtctcactggaaagcacgaaaccgcagacatcaacacattctcttggggagtcgcgaaccgtggagcgtcagtgagagtgggacgtgacacagagaaggaaggtaaagggtacttcgaagacagaaggccagcttctaacatggatccttacgttgtcacctccatgatcgctgagacgaccatactcggttga

SEQ ID NO: 7 - protein, 364 aa, Arabidopsis thaliana
  SITE (1)..(9): amino terminal 9 amino acids artificial vector encoded sequence
MVDLRNRRTSMSLLSDLVNLNLTDATGKIIAEYIWIGGSGMDIRSKARTLPGPVTDPSKLPKWNYDGSSTGQAAGEDSEVILYPQAIFKDPFRKGNNILVMCDAYTPAGDPIPTNKRHNAAKIFSHPDVAKEEPWYGIEQEYTLMQKDVNWPIGWPVGGYPGPQGPYYCGVGADKAIGRDIVDAHYKACLYAGIGISGINGEVMPGQWEFQVGPVEGISSGDQVWVARYLLERITEISGVIVSFDPKPVPGDWNGAGAHCNYSTKTMRNDGGLEVIKKAIGKLQLKHKEHIAAYGEGNERRLTGKHETADINTFSWGVANRGASVRVGRDTEKEGKGYFEDRRPASNMDPYVVTSMIAETTILG

SEQ ID NO: 8 - DNA, 1817 bp, Artificial Sequence
  Synthetic plasmid vector sequence including Vitis vinifera GPT coding sequence
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgcagctctctcaatgtacctggacattcccagagttgcttaaaagaccagcctttttaaggaggagtattgatagtatttcgagtagaagtaggtccagctccaagtatccatctttcatggcgtccgcatcaacggtctccgctccaaatacggaggctgagcagacccataacccccctcaacctctacaggttgcaaagcgcttggagaaattcaaaacaacaatctttactcaaatgagcatgcttgccatcaaacatggagcaataaaccttggccaagggtttcccaactttgatggtcctgagtttgtcaaagaagcagcaattcaagccattaaggatgggaaaaaccaatatgctcgtggatatggagttcctgatctcaactctgctgttgctgatagattcaagaaggatacaggactcgtggtggaccccgagaaggaagttactgttacttctggatgtacagaagcaattgctgctactatgctaggcttgataaatcctggtgatgaggtgatcctctttgctccattttatgattcctatgaagccactctatccatggctggtgcccaaataaaatccatcactttacgtcctccggattttgctgtgcccatggatgagctcaagtctgcaatctcaaagaatacccgtgcaatccttataaacactccccataaccccacaggaaagatgttcacaagggaggaactgaatgtgattgcatccctctgcattgagaatgatgtgttggtgtttactgatgaagtttacgacaagttggctttcgaaatggatcacatttccatggcttctcttcctgggatgtacgagaggaccgtgactatgaattccttagggaaaactttctccctgactggatggaagattggttggacagtagctcccccacacctgacatggggagtgaggcaagcccactcattcctcacgtttgctacctgcaccccaatgcaatgggcagctgcaacagccctccgggccccagactcttactatgaagagctaaagagagattacagtgcaaagaaggcaatcctggtggagggattgaaggctgtcggtttcagggtatacccatcaagtgggacctattttgtggtggtggatcacaccccatttgggttgaaagacgatattgcgttttgtgagtatctgatcaaggaagttggggtggtagcaattccgacaagcgttttctacttacacccagaagatggaaagaaccttgtgaggtttaccttctgtaaagacgagggaactctgagagctgcagttgaaaggatgaaggagaaactgaagcctaaacaataggggcacgtga

SEQ ID NO: 9 - protein, 459 aa, Vitis vinifera
MVDLRNRRTSMQLSQCTWTFPELLKRPAFLRRSIDSISSRSRSSSKYPSFMASASTVSAPNTEAEQTHNPPQPLQVAKRLEKFKTTIFTQMSMLAIKHGAINLGQGFPNFDGPEFVKEAAIQAIKDGKNQYARGYGVPDLNSAVADRFKKDTGLVVDPEKEVTVTSGCTEAIAATMLGLINPGDEVILFAPFYDSYEATLSMAGAQIKSITLRPPDFAVPMDELKSAISKNTRAILINTPHNPTGKMFTREELNVIASLCIENDVLVFTDEVYDKLAFEMDHISMASLPGMYERTVTMNSLGKTFSLTGWKIGWTVAPPHLTWGVRQAHSFLTFATCTPMQWAAATALRAPDSYYEELKRDYSAKKAILVEGLKAVGFRVYPSSGTYFVVVDHTPFGLKDDIAFCEYLIKEVGVVAIPTSVFYLHPEDGKNLVRFTFCKDEGTLRAAVERMKEKLKPKQ

SEQ ID NO: 10 - DNA, 1446 bp, Artificial Sequence
  Synthetic DNA encoding Oryza sativa GPT protein, codons optimized for expression in E. coli
atgtggatgaacctggcaggctttctggcaaccccggcaaccgcaaccgcaacccgtcatgaaatgccgctgaacccgagcagcagcgcgagctttctgctgagcagcctgcgtcgtagcctggtggcgagcctgcgtaaagcgagcccggcagcagcagcagcactgagcccgatggcaagcgcaagcaccgtggcagcagaaaacggtgcagcaaaagcagcagcagaaaaacagcagcagcagccggtgcaggtggcgaaacgtctggaaaaatttaaaaccaccatttttacccagatgagcatgctggcgattaaacatggcgcgattaacctgggccagggctttccgaactttgatggcccggattttgtgaaagaagcggcgattcaggcgattaacgcgggcaaaaaccagtatgcgcgtggctatggcgtgccggaactgaacagcgcgattgcggaacgttttctgaaagatagcggcctgcaggtggatccggaaaaagaagtgaccgtgaccagcggctgcaccgaagcgattgcggcgaccattctgggcctgattaacccgggcgatgaagtgattctgtttgcgccgttttatgatagctatgaagcgaccctgagcatggcgggcgcgaacgtgaaagcgattaccctgcgtccgccggattttagcgtgccgctggaagaactgaaagcggccgtgagcaaaaacacccgtgcgattatgattaacaccccgcataacccgaccggcaaaatgtttacccgtgaagaactggaatttattgcgaccctgtgcaaagaaaacgatgtgctgctgtttgcggatgaagtgtatgataaactggcgtttgaagcggatcatattagcatggcgagcattccgggcatgtatgaacgtaccgtgaccatgaacagcctgggcaaaacctttagcctgaccggctggaaaattggctgggcgattgcgccgccgcatctgacctggggcgtgcgtcaggcacatagctttctgacctttgcaacctgcaccccgatgcaggcagccgccgcagcagcactgcgtgcaccggatagctattatgaagaactgcgtcgtgattatggcgcgaaaaaagcgctgctggtgaacggcctgaaagatgcgggctttattgtgtatccgagcagcggcacctattttgtgatggtggatcataccccgtttggctttgataacgatattgaattttgcgaatatctgattcgtgaagtgggcgtggtggcgattccgccgagcgtgttttatctgaacccggaagatggcaaaaacctggtgcgttttaccttttgcaaagatgatgaaaccctgcgtgcggcggtggaacgtatgaaaaccaaactgcgtaaaaaaaagcttgcggccgcactcgagcaccaccaccaccaccactga

SEQ ID NO: 11 - protein, 481 aa, Artificial Sequence
  Oryza sativa GPT protein sequence with amino- and carboxyl-terminal vector sequences
MWMNLAGFLATPATATATRHEMPLNPSSSASFLLSSLRRSLVASLRKASPAAAAALSPMASASTVAAENGAAKAAAEKQQQQPVQVAKRLEKFKTTIFTQMSMLAIKHGAINLGQGFPNFDGPDFVKEAAIQAINAGKNQYARGYGVPELNSAIAERFLKDSGLQVDPEKEVTVTSGCTEAIAATILGLINPGDEVILFAPFYDSYEATLSMAGANVKAITLRPPDFSVPLEELKAAVSKNTRAIMINTPHNPTGKMFTREELEFIATLCKENDVLLFADEVYDKLAFEADHISMASIPGMYERTVTMNSLGKTFSLTGWKIGWAIAPPHLTWGVRQAHSFLTFATCTPMQAAAAAALRAPDSYYEELRRDYGAKKALLVNGLKDAGFIVYPSSGTYFVMVDHTPFGFDNDIEFCEYLIREVGVVAIPPSVFYLNPEDGKNLVRFTFCKDDETLRAAVERMKTKLRKKKLAAALEHHHHHH

SEQ ID NO: 12 - DNA, 1251 bp, Artificial Sequence
  Synthetic DNA encoding Glycine max GPT protein, codons optimized for expression in E. coli
atgcatcatcaccatcaccatggtaagcctatccctaaccctctcctcggtctcgattctacggaaaacctgtattttcagggaattgatcccttcaccgcgaaacgtctggaaaaatttcagaccaccatttttacccagatgagcctgctggcgattaaacatggcgcgattaacctgggccagggctttccgaactttgatggcccggaatttgtgaaagaagcggcgattcaggcgattcgtgatggcaaaaaccagtatgcgcgtggctatggcgtgccggatctgaacattgcgattgcggaacgttttaaaaaagataccggcctggtggtggatccggaaaaagaaattaccgtgaccagcggctgcaccgaagcgattgcggcgaccatgattggcctgattaacccgggcgatgaagtgattatgtttgcgccgttttatgatagctatgaagcgaccctgagcatggcgggcgcgaaagtgaaaggcattaccctgcgtccgccggattttgcggtgccgctggaagaactgaaaagcaccattagcaaaaacacccgtgcgattctgattaacaccccgcataacccgaccggcaaaatgtttacccgtgaagaactgaactgcattgcgagcctgtgcattgaaaacgatgtgctggtgtttaccgatgaagtgtatgataaactggcgtttgatatggaacatattagcatggcgagcctgccgggcatgtttgaacgtaccgtgaccctgaacagcctgggcaaaacctttagcctgaccggctggaaaattggctgggcgattgcgccgccgcatctgagctggggcgtgcgtcaggcgcatgcgtttctgacctttgcaaccgcacatccgtttcagtgcgcagcagcagcagcactgcgtgcaccggatagctattatgtggaactgaaacgtgattatatggcgaaacgtgcgattctgattgaaggcctgaaagcggtgggctttaaagtgtttccgagcagcggcacctattttgtggtggtggatcataccccgtttggcctggaaaacgatgtggcgttttgcgaatatctggtgaaagaagtgggcgtggtggcgattccgaccagcgtgttttatctgaacccggaagaaggcaaaaacctggtgcgttttaccttttgcaaagatgaagaaaccattcgtagcgcggtggaacgtatgaaagcgaaactgcgtaaagtcgactaa

SEQ ID NO: 13 - protein, 416 aa, Artificial Sequence
  Glycine max GPT amino acid sequence and amino-terminal vector sequence
MHHHHHHGKPIPNPLLGLDSTENLYFQGIDPFTAKRLEKFQTTIFTQMSLLAIKHGAINLGQGFPNFDGPEFVKEAAIQAIRDGKNQYARGYGVPDLNIAIAERFKKDTGLVVDPEKEITVTSGCTEAIAATMIGLINPGDEVIMFAPFYDSYEATLSMAGAKVKGITLRPPDFAVPLEELKSTISKNTRAILINTPHNPTGKMFTREELNCIASLCIENDVLVFTDEVYDKLAFDMEHISMASLPGMFERTVTLNSLGKTFSLTGWKIGWAIAPPHLSWGVRQAHAFLTFATAHPFQCAAAAALRAPDSYYVELKRDYMAKRAILIEGLKAVGFKVFPSSGTYFVVVDHTPFGLENDVAFCEYLVKEVGVVAIPTSVFYLNPEEGKNLVRFTFCKDEETIRSAVERMKAKLRKVD

SEQ ID NO: 14 - DNA, 1278 bp, Hordeum vulgare
atggtagatctgaggaaccgacgaactagtatggcatccgcccccgcctccgcctccgcggccctctccaccgccgcccccgccgacaacggggccgccaagcccacggagcagcggccggtacaggtggctaagcgattggagaagttcaaaacaacaattttcacacagatgagcatgctcgcagtgaagcatggagcaataaaccttggacaggggtttcccaattttgatggccctgactttgtcaaagatgctgctattgaggctatcaaagctggaaagaatcagtatgcaagaggatatggtgtgcctgaattgaactcagctgttgctgagagatttctcaaggacagtggattgcacatcgatcctgataaggaagttactgttacatctgggtgcacagaagcaatagctgcaacgatattgggtctgatcaaccctggggatgaagtcatactgtttgctccattctatgattcttatgaggctacactgtccatggctggtgcgaatgtcaaagccattacactccgccctccggactttgcagtccctcttgaagagctaaaggctgcagtctcgaagaataccagagcaataatgattaatacacctcacaaccctaccgggaaaatgttcacaagggaggaacttgagttcattgctgatctctgcaaggaaaatgacgtgttgctctttgccgatgaggtctacgacaagctggcgtttgaggcggatcacatatcaatggcttctattcctggcatgtatgagaggaccgtcactatgaactccctggggaagacgttctccttgaccggatggaagatcggctgggcgatagcaccaccgcacctgacatggggcgtaaggcaggcacactccttcctcacattcgccacctccacgccgatgcaatcagcagcggcggcggccctgagagcaccggacagctactttgaggagctgaagagggactacggcgcaaagaaagcgctgctggtggacgggctcaaggcggcgggcttcatcgtctacccttcgagcggaacctacttcatcatggtcgaccacaccccgttcgggttcgacaacgacgtcgagttctgcgagtacttgatccgcgaggtcggcgtcgtggccatcccgccaagcgtgttctacctgaacccggaggacgggaagaacctggtgaggttcaccttctgcaaggacgacgacacgctaagggcggcggtggacaggatgaaggccaagctcaggaagaaatga

SEQ ID NO: 15 - protein, 425 aa, Hordeum vulgare
MVDLRNRRTSMASAPASASAALSTAAPADNGAAKPTEQRPVQVAKRLEKFKTTIFTQMSMLAVKHGAINLGQGFPNFDGPDFVKDAAIEAIKAGKNQYARGYGVPELNSAVAERFLKDSGLHIDPDKEVTVTSGCTEAIAATILGLINPGDEVILFAPFYDSYEATLSMAGANVKAITLRPPDFAVPLEELKAAVSKNTRAIMINTPHNPTGKMFTREELEFIADLCKENDVLLFADEVYDKLAFEADHISMASIPGMYERTVTMNSLGKTFSLTGWKIGWAIAPPHLTWGVRQAHSFLTFATSTPMQSAAAAALRAPDSYFEELKRDYGAKKALLVDGLKAAGFIVYPSSGTYFIMVDHTPFGFDNDVEFCEYLIREVGVVAIPPSVFYLNPEDGKNLVRFTFCKDDDTLRAAVDRMKAKLRKK

SEQ ID NO: 16 - DNA, 1200 bp, Artificial Sequence
  Synthetic DNA encoding Danio rerio GPT protein, codons optimized for expression in E. coli, including 5′ and 3′ vector sequences
atgtccgtggcgaaacgtctggaaaaatttaaaaccaccatttttacccagatgagcatgctggcgattaaacatggcgcgattaacctgggccagggctttccgaactttgatggcccggattttgtgaaagaagcggcgattcaggcgattcgtgatggcaacaaccagtatgcgcgtggctatggcgtgccggatctgaacattgcgattagcgaacgttataaaaaagataccggcctggcggtggatccggaaaaagaaattaccgtgaccagcggctgcaccgaagcgattgcggcgaccgtgctgggcctgattaacccgggcgatgaagtgattgtgtttgcgccgttttatgatagctatgaagcgaccctgagcatggcgggcgcgaaagtgaaaggcattaccctgcgtccgccggattttgcgctgccgattgaagaactgaaaagcaccattagcaaaaacacccgtgcgattctgctgaacaccccgcataacccgaccggcaaaatgtttaccccggaagaactgaacaccattgcgagcctgtgcattgaaaacgatgtgctggtgtttagcgatgaagtgtatgataaactggcgtttgatatggaacatattagcattgcgagcctgccgggcatgtttgaacgtaccgtgaccatgaacagcctgggcaaaacctttagcctgaccggctggaaaattggctgggcgattgcgccgccgcatctgacctggggcgtgcgtcaggcgcatgcgtttctgacctttgcaaccagcaacccgatgcagtgggcagcagcagtggcactgcgtgcaccggatagctattataccgaactgaaacgtgattatatggcgaaacgtagcattctggtggaaggcctgaaagcggtgggctttaaagtgtttccgagcagcggcacctattttgtggtggtggatcataccccgtttggccatgaaaacgatattgcgttttgcgaatatctggtgaaagaagtgggcgtggtggcgattccgaccagcgtgttttatctgaacccggaagaaggcaaaaacctggtgcgttttaccttttgcaaagatgaaggcaccctgcgtgcggcggtggatcgtatgaaagaaaaactgcgtaaagtcgacaagcttgcggccgcactcgagcaccaccaccaccaccactga

SEQ ID NO: 17 - protein, 399 aa, Danio rerio
  MISC_FEATURE (1)..(399): Amino- and carboxy-terminal amino acids shown
MSVAKRLEKFKTTIFTQMSMLAIKHGAINLGQGFPNFDGPDFVKEAAIQAIRDGNNQYARGYGVPDLNIAISERYKKDTGLAVDPEKEITVTSGCTEAIAATVLGLINPGDEVIVFAPFYDSYEATLSMAGAKVKGITLRPPDFALPIEELKSTISKNTRAILLNTPHNPTGKMFTPEELNTIASLCIENDVLVFSDEVYDKLAFDMEHISIASLPGMFERTVTMNSLGKTFSLTGWKIGWAIAPPHLTWGVRQAHAFLTFATSNPMQWAAAVALRAPDSYYTELKRDYMAKRSILVEGLKAVGFKVFPSSGTYFVVVDHTPFGHENDIAFCEYLVKEVGVVAIPTSVFYLNPEEGKNLVRFTFCKDEGTLRAAVDRMKEKLRKVDKLAAALEHHHHHH

SEQ ID NO: 18 - DNA, 1236 bp, Arabidopsis thaliana
atggccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 19 - protein, 411 aa, Arabidopsis thaliana
MAKIHRPIGATMTTVSTQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 20 - DNA, 1194 bp, Arabidopsis thaliana
atggcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 21 - protein, 397 aa, Arabidopsis thaliana
MATQNESTQKPVQVAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 22 - DNA, 1680 bp, Lycopersicon esculentum
ggtaccgtttgaatcctccttaaagtttttctctggagaaactgtagtaattttactttgttgtgttcccttcatcttttgaattaatggcatttgttttaatactaatctgcttctgaaacttgtaatgtatgtatatcagtttcttataatttatccaagtaatatcttccattctctatgcaattgcctgcataagctcgacaaaagagtacatcaacccctcctcctctggactactctagctaaacttgaatttccccttaagattatgaaattgatatatccttaacaaacgactccttctgttggaaaatgtagtacttgtctttcttcttttgggtatatatagtttatatacaccatactatgtacaacatccaagtagagtgaaatggatacatgtacaagacttatttgattgattgatgacttgagttgccttaggagtaacaaattcttaggtcaataaatcgttgatttgaaattaatctctctgtcttagacagataggaattatgacttccaatggtccagaaagcaaagttcgcactgagggtatacttggaattgagacttgcacaggtccagaaaccaaagttcccatcgagctctaaaatcacatctttggaatgaaattcaattagagataagttgcttcatagcataggtaaaatggaagatgtgaagtaacctgcaataatcagtgaaatgacattaatacactaaatacttcatatgtaattatcctttccaggttaacaatactctataaagtaagaattatcagaaatgggctcatcaaacttttgtactatgtatttcatataaggaagtataactatacataagtgtatacacaactttattcctattttgtaaaggtggagagactgttttcgatggatctaaagcaatatgtctataaaatgcattgatataataattatctgagaaaatccagaattggcgttggattatttcagccaaatagaagtttgtaccatacttgttgattccttctaagttaaggtgaagtatcattcataaacagttttccccaaagtactactcaccaagtttccctttgtagaattaacagttcaaatatatggcgcagaaattactctatgcccaaaaccaaacgagaaagaaacaaaatacaggggttgcagactttattttcgtgttagggtgtgttttttcatgtaattaatcaaaaaatattatgacaaaaacatttatacatatttttactcaacactctgggtatcagggtgggttgtgttcgacaatcaatatggaaaggaagtattttccttatttttttagttaatattttcagttataccaaacataccttgtgatattatttttaaaaatgaaaaactcgtcagaaagaaaaagcaaaagcaacaaaaaaattgcaagtattttttaaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatgg

SEQ ID NO: 23 - DNA, 1230 bp, Phyllostachys bambusoides
atggcctccgcggccgtctccaccgtcgccaccgccgccgacggcgtcgcgaagccgacggagaagcagccggtacaggtcgcaaagcgtttggaaaagtttaagacaacaattttcacacagatgagcatgcttgccatcaagcatggagcaataaacctcggccagggctttccgaattttgatggccctgactttgtgaaagaagctgctattcaagctatcaatgctgggaagaatcagtatgcaagaggatatggtgtgcctgaactgaactcggctgttgctgaaaggttcctgaaggacagtggcttgcaagtcgatcccgagaaggaagttactgtcacatctgggtgcacggaagcgatagctgcaacgatattgggtcttatcaaccctggcgatgaagtgatcttgtttgctccattctatgattcatacgaggctacgctgtcgatggctggtgccaatgtaaaagccattactctccgtcctccagattttgcagtccctcttgaggagctaaaggccacagtctctaagaacaccagagcgataatgataaacacaccacacaatcctactgggaaaatgttttctagggaagaacttgaattcattgctactctctgcaagaaaaatgatgtgttgctttttgctgatgaggtctatgacaagttggcatttgaggcagatcatatatcaatggcttctattcctggcatgtatgagaggactgtgactatgaactctctggggaagacattctctctaacaggatggaagatcggttgggcaatagcaccaccacacctgacatggggtgtaaggcaggcacactcattcctcacatttgccacctgcacaccaatgcaatcggcggcggcggcggctcttagagcaccagatagctactatggggagctgaagagggattacggtgcaaagaaagcgatactagtcgacggactcaaggctgcaggttttattgtttacccttcaagtggaacatactttgtcatggtcgatcacaccccgtttggtttcgacaatgatattgagttctgcgagtatttgatccgcgaagtcggtgttgtcgccataccaccaagcgtattttatctcaaccctgaggatgggaagaacttggtgaggttcaccttctgcaaggatgatgatacgctgagagccgcagttgagaggatgaagacaaagctcaggaaaaaatga

SEQ ID NO: 24 - protein, 409 aa, Phyllostachys bambusoides
MASAAVSTVATAADGVAKPTEKQPVQVAKRLEKFKTTIFTQMSMLAIKHGAINLGQGFPNFDGPDFVKEAAIQAINAGKNQYARGYGVPELNSAVAERFLKDSGLQVDPEKEVTVTSGCTEAIAATILGLINPGDEVILFAPFYDSYEATLSMAGANVKAITLRPPDFAVPLEELKATVSKNTRAIMINTPHNPTGKMFSREELEFIATLCKKNDVLLFADEVYDKLAFEADHISMASIPGMYERTVTMNSLGKTFSLTGWKIGWAIAPPHLTWGVRQAHSFLTFATCTPMQSAAAAALRAPDSYYGELKRDYGAKKAILVDGLKAAGFIVYPSSGTYFVMVDHTPFGFDNDIEFCEYLIREVGVVAIPPSVFYLNPEDGKNLVRFTFCKDDDTLRAAVERMKTKLRKK

SEQ ID NO: 25 - DNA, 1858 bp, Oryza sativa
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgaatctggccggctttctcgccacgcccgcgaccgcgaccgcgacgcggcatgagatgccgttaaatccctcctcctccgcctccttcctcctctcctcgctccgccgctcgctcgtcgcgtcgctccggaaggcctcgccggcggcggccgcggcgctctcccccatggcctccgcgtccaccgtcgccgccgagaacggcgccgccaaggcggcggcggagaagcagcagcagcagcctgtgcaggttgcaaagcggttggaaaagtttaagacgaccattttcacacagatgagtatgcttgccatcaagcatggagcaataaaccttggccagggttttccgaatttcgatggccctgactttgtaaaagaggctgctattcaagctatcaatgctgggaagaatcagtacgcaagaggatatggtgtgcctgaactgaactcagctattgctgaaagattcctgaaggacagcggactgcaagtcgatccggagaaggaagttactgtcacatctggatgcacagaagctatagctgcaacaattttaggtctaattaatccaggcgatgaagtgatattgtttgctccattctatgattcatatgaggctaccctgtcaatggctggtgccaacgtaaaagccattactctccgtcctccagatttttcagtccctcttgaagagctaaaggctgcagtctcgaagaacaccagagctattatgataaacaccccgcacaatcctactgggaaaatgtttacaagggaagaacttgagtttattgccactctctgcaaggaaaatgatgtgctgctttttgctgatgaggtctacgacaagttagcttttgaggcagatcatatatcaatggcttctattcctggcatgtatgagaggaccgtgaccatgaactctcttgggaagacattctctcttacaggatggaagatcggttgggcaatcgcaccgccacacctgacatggggtgtaaggcaggcacactcattcctcacgtttgcgacctgcacaccaatgcaagcagctgcagctgcagctctgagagcaccagatagctactatgaggaactgaggagggattatggagctaagaaggcattgctagtcaacggactcaaggatgcaggtttcattgtctatccttcaagtggaacatacttcgtcatggtcgaccacaccccatttggtttcgacaatgatattgagttctgcgagtatttgattcgcgaagtcggtgttgtcgccataccacctagtgtattttatctcaaccctgaggatgggaagaacttggtgaggttcaccttttgcaaggatgatgagacgctgagagccgcggttgagaggatgaagacaaagctcaggaaaaaatga

SEQ ID NO: 26 - DNA, 1724 bp, Artificial Sequence
  Synthetic DNA encoding Hordeum vulgare GPT protein
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatggcatccgcccccgcctccgcctccgcggccctctccaccgccgcccccgccgacaacggggccgccaagcccacggagcagcggccggtacaggtggctaagcgattggagaagttcaaaacaacaattttcacacagatgagcatgctcgcagtgaagcatggagcaataaaccttggacaggggtttcccaattttgatggccctgactttgtcaaagatgctgctattgaggctatcaaagctggaaagaatcagtatgcaagaggatatggtgtgcctgaattgaactcagctgttgctgagagatttctcaaggacagtggattgcacatcgatcctgataaggaagttactgttacatctgggtgcacagaagcaatagctgcaacgatattgggtctgatcaaccctggggatgaagtcatactgtttgctccattctatgattcttatgaggctacactgtccatggctggtgcgaatgtcaaagccattacactccgccctccggactttgcagtccctcttgaagagctaaaggctgcagtctcgaagaataccagagcaataatgattaatacacctcacaaccctaccgggaaaatgttcacaagggaggaacttgagttcattgctgatctctgcaaggaaaatgacgtgttgctctttgccgatgaggtctacgacaagctggcgtttgaggcggatcacatatcaatggcttctattcctggcatgtatgagaggaccgtcactatgaactccctggggaagacgttctccttgaccggatggaagatcggctgggcgatagcaccaccgcacctgacatggggcgtaaggcaggcacactccttcctcacattcgccacctccacgccgatgcaatcagcagcggcggcggccctgagagcaccggacagctactttgaggagctgaagagggactacggcgcaaagaaagcgctgctggtggacgggctcaaggcggcgggcttcatcgtctacccttcgagcggaacctacttcatcatggtcgaccacaccccgttcgggttcgacaacgacgtcgagttctgcgagtacttgatccgcgaggtcggcgtcgtggccatcccgccaagcgtgttctacctgaacccggaggacgggaagaacctggtgaggttcaccttctgcaaggacgacgacacgctaagggcggcggtggacaggatgaaggccaagctcaggaagaaatgattgaggggcgcacgtgtga

SEQ ID NO: 27 - DNA, 1868 bp, Artificial Sequence
  Synthetic DNA encoding Arabidopsis thaliana GPT protein
catggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 28 - DNA, 1780 bp, Artificial Sequence
  Synthetic DNA encoding Arabidopsis thaliana GPT protein
aaaaaagaaaaaaaaaacatatcttgtttgtcagtatgggaagtttgagataaggacgagtgaggggttaaaattcagtggccattgattttgtaatgccaagaaccacaaaatccaatggttaccattcctgtaagatgaggtttgctaactctttttgtccgttagataggaagccttatcactatatatacaaggcgtcctaataacctcttagtaaccaattatttcagcaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtatgtacctggacataaatggtgtgatgatcaaacagtttagcttcaaagcctctcttctcccattctcttctaatttccgacaaagctccgccaaaatccatcgtcctatcggagccaccatgaccacagtttcgactcagaacgagtctactcaaaaacccgtccaggtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 29 - DNA, 1155 bp, Arabidopsis thaliana
gtggcgaagagattagagaagttcaagactactattttcactcaaatgagcatattggcagttaaacatggagcgatcaatttaggccaaggctttcccaatttcgacggtcctgattttgttaaagaagctgcgatccaagctattaaagatggtaaaaaccagtatgctcgtggatacggcattcctcagctcaactctgctatagctgcgcggtttcgtgaagatacgggtcttgttgttgatcctgagaaagaagttactgttacatctggttgcacagaagccatagctgcagctatgttgggtttaataaaccctggtgatgaagtcattctctttgcaccgttttatgattcctatgaagcaacactctctatggctggtgctaaagtaaaaggaatcactttacgtccaccggacttctccatccctttggaagagcttaaagctgcggtaactaacaagactcgagccatccttatgaacactccgcacaacccgaccgggaagatgttcactagggaggagcttgaaaccattgcatctctctgcattgaaaacgatgtgcttgtgttctcggatgaagtatacgataagcttgcgtttgaaatggatcacatttctatagcttctcttcccggtatgtatgaaagaactgtgaccatgaattccctgggaaagactttctctttaaccggatggaagatcggctgggcgattgcgccgcctcatctgacttggggagttcgacaagcacactcttacctcacattcgccacatcaacaccagcacaatgggcagccgttgcagctctcaaggcaccagagtcttacttcaaagagctgaaaagagattacaatgtgaaaaaggagactctggttaagggtttgaaggaagtcggatttacagtgttcccatcgagcgggacttactttgtggttgctgatcacactccatttggaatggagaacgatgttgctttctgtgagtatcttattgaagaagttggggtcgttgcgatcccaacgagcgtcttttatctgaatccagaagaagggaagaatttggttaggtttgcgttctgtaaagacgaagagacgttgcgtggtgcaattgagaggatgaagcagaagcttaagagaaaagtctga

SEQ ID NO: 30 - protein, 384 aa, Arabidopsis thaliana
VAKRLEKFKTTIFTQMSILAVKHGAINLGQGFPNFDGPDFVKEAAIQAIKDGKNQYARGYGIPQLNSAIAARFREDTGLVVDPEKEVTVTSGCTEAIAAAMLGLINPGDEVILFAPFYDSYEATLSMAGAKVKGITLRPPDFSIPLEELKAAVTNKTRAILMNTPHNPTGKMFTREELETIASLCIENDVLVFSDEVYDKLAFEMDHISIASLPGMYERTVTMNSLGKTFSLTGWKIGWAIAPPHLTWGVRQAHSYLTFATSTPAQWAAVAALKAPESYFKELKRDYNVKKETLVKGLKEVGFTVFPSSGTYFVVADHTPFGMENDVAFCEYLIEEVGVVAIPTSVFYLNPEEGKNLVRFAFCKDEETLRGAIERMKQKLKRKV

SEQ ID NO: 31 - protein, 384 aa, Vitis vinifera
VAKRLEKFKTTIFTQMSMLAIKHGAINLGQGFPNFDGPEFVKEAAIQAIKDGKNQYARGYGVPDLNSAVADRFKKDTGLVVDPEKEVTVTSGCTEAIAATMLGLINPGDEVILFAPFYDSYEATLSMAGAQIKSITLRPPDFAVPMDELKSAISKNTRAILINTPHNPTGKMFTREELNVIASLCIENDVLVFTDEVYDKLAFEMDHISMASLPGMYERTVTMNSLGKTFSLTGWKIGWTVAPPHLTWGVRQAHSFLTFATCTPMQWAAATALRAPDSYYEELKRDYSAKKAILVEGLKAVGFRVYPSSGTYFVVVDHTPFGLKDDIAFCEYLIKEVGVVAIPTSVFYLHPEDGKNLVRFTFCKDEGTLRAAVERMKEKLKPKQ

SEQ ID NO: 32 - protein, 383 aa, Oryza sativa
VAKRLEKFKTTIFTQMSMLAIKHGAINLGQGFPNFDGPDFVKEAAIQAINAGKNQYARGYGVPELNSAIAERFLKDSGLQVDPEKEVTVTSGCTEAIAATILGLINPGDEVILFAPFYDSYEATLSMAGANVKAITLRPPDFSVPLEELKAAVSKNTRAIMINTPHNPTGKMFTREELEFIATLCKENDVLLFADEVYDKLAFEADHISMASIPGMYERTVTMNSLGKTFSLTGWKIGWAIAPPHLTWGVRQAHSFLTFATCTPMQAAAAAALRAPDSYYEELRRDYGAKKALLVNGLKDAGFIVYPSSGTYFVMVDHTPFGFDNDIEFCEYLIREVGVVAIPPSVFYLNPEDGKNLVRFTFCKDDETLRAAVERMKTKLRKK

SEQ ID NO: 33 - protein, 383 aa, Glycine max
AKRLEKFQTTIFTQMSLLAIKHGAINLGQGFPNFDGPEFVKEAAIQAIRDGKNQYARGYGVPDLNIAIAERFKKDTGLVVDPEKEITVTSGCTEAIAATMIGLINPGDEVIMFAPFYDSYEATLSMAGAKVKGITLRPPDFAVPLEELKSTISKNTRAILINTPHNPTGKMFTREELNCIASLCIENDVLVFTDEVYDKLAFDMEHISMASLPGMFERTVTLNSLGKTFSLTGWKIGWAIAPPHLSWGVRQAHAFLTFATAHPFQCAAAAALRAPDSYYVELKRDYMAKRAILIEGLKAVGFKVFPSSGTYFVVVDHTPFGLENDVAFCEYLVKEVGVVAIPTSVFYLNPEEGKNLVRFTFCKDEETIRSAVERMKAKLRKVD

SEQ ID NO: 34 - protein, 383 aa, Hordeum vulgare
VAKRLEKFKTTIFTQMSMLAVKHGAINLGQGFPNFDGPDFVKDAAIEAIKAGKNQYARGYGVPELNSAVAERFLKDSGLHIDPDKEVTVTSGCTEAIAATILGLINPGDEVILFAPFYDSYEATLSMAGANVKAITLRPPDFAVPLEELKAAVSKNTRAIMINTPHNPTGKMFTREELEFIADLCKENDVLLFADEVYDKLAFEADHISMASIPGMYERTVTMNSLGKTFSLTGWKIGWAIAPPHLTWGVRQAHSFLTFATSTPMQSAAAAALRAPDSYFEELKRDYGAKKALLVDGLKAAGFIVYPSSGTYFIMVDHTPFGFDNDVEFCEYLIREVGVVAIPPSVFYLNPEDGKNLVRFTFCKDDDTLRAAVDRMKAKLRKK

SEQ ID NO: 35 - protein, 382 aa, Danio rerio
VAKRLEKFKTTIFTQMSMLAIKHGAINLGQGFPNFDGPDFVKEAAIQAIRDGNNQYARGYGVPDLNIAISERYKKDTGLAVDPEKEITVTSGCTEAIAATVLGLINPGDEVIVFAPFYDSYEATLSMAGAKVKGITLRPPDFALPIEELKSTISKNTRAILLNTPHNPTGKMFTPEELNTIASLCIENDVLVFSDEVYDKLAFDMEHISIASLPGMFERTVTMNSLGKTFSLTGWKIGWAIAPPHLTWGVRQAHAFLTFATSNPMQWAAAVALRAPDSYYTELKRDYMAKRSILVEGLKAVGFKVFPSSGTYFVVVDHTPFGHENDIAFCEYLVKEVGVVAIPTSVFYLNPEEGKNLVRFTFCKDEGTLRAAVDRMKEKLRK

SEQ ID NO: 36 - protein, 383 aa, Phyllostachys bambusoides
VAKRLEKFKTTIFTQMSMLAIKHGAINLGQGFPNFDGPDFVKEAAIQAINAGKNQYARGYGVPELNSAVAERFLKDSGLQVDPEKEVTVTSGCTEAIAATILGLINPGDEVILFAPFYDSYEATLSMAGANVKAITLRPPDFAVPLEELKATVSKNTRAIMINTPHNPTGKMFSREELEFIATLCKKNDVLLFADEVYDKLAFEADHISMASIPGMYERTVTMNSLGKTFSLTGWKIGWAIAPPHLTWGVRQAHSFLTFATCTPMQSAAAAALRAPDSYYGELKRDYGAKKAILVDGLKAAGFIVYPSSGTYFVMVDHTPFGFDNDIEFCEYLIREVGVVAIPPSVFYLNPEDGKNLVRFTFCKDDDTLRAAVERMKTKLRKK

SEQ ID NO: 37 - DNA, 34 bp, Artificial Sequence
  Synthetic primer sequence
cccatcgatgtacctggacataaatggtgtgatg

SEQ ID NO: 38 - DNA, 37 bp, Artificial Sequence
  Synthetic Primer Sequence
gatggtacctcagacttttctcttaagcttctgcttc